(19) United States
(12) Patent Application Publication
(10) Pub. No.: US 2023/0332120 A1
(43) Pub. Date: Oct. 19, 2023

(54) CAS9 PROTEINS WITH ENHANCED SPECIFICITY AND USES THEREOF

(71) Applicant: GENECKER CO., LTD., Seoul (KR)

(72) Inventor: Sung-Hyeok YE, Gaithersburg, MD (US)

(73) Assignee: GENECKER CO., LTD., Seoul (KR)

(21) Appl. No.: 18/153,180

(22) Filed: Jan. 11, 2023

Related U.S. Application Data
(60) Provisional application No. 63/298,822, filed on Jan. 12, 2022.

(51) Int. Cl. C12N 9/22; C12N 15/11; C12Q 1/6886

(52) U.S. Cl. CPC C12N 9/22; C12N 15/111; C12Q 1/6886; C12N 2310/20; C12Q 2600/156

(57) ABSTRACT

The present disclosure relates to modified Cas9 proteins with enhanced specificity. In some aspects, the modified Cas9 proteins allow for reduced off-target effects during gene editing. The present disclosure also relates to the use of such modified Cas9 proteins to diagnose and/or treat a disease or disorder in a subject.

### CROSS-REFERENCE TO RELATED APPLICATION

This application claims the priority benefit of U.S. Provisional Application No. 63/298,822, filed on Jan. 12, 2022, which is herein incorporated by reference in its entirety.

### REFERENCE TO SEQUENCE LISTING SUBMITTED ELECTRONICALLY

The content of the electronically submitted sequence listing (Name: 4603_0010001_Seqlisting_ST26.xml, Size: 339,518 bytes; and Date of Creation: Jan. 10, 2023) submitted in this application is incorporated herein by reference in its entirety.

### FIELD OF THE DISCLOSURE

The present disclosure provides Cas9 proteins that have been modified to exhibit enhanced specificity (or fidelity), including compositions, polynucleotides, vectors, cells, and kits relating to such Cas9 proteins. The present disclosure also provides methods of producing and using the modified Cas9 proteins in a wide range of clinical settings (e.g., both therapeutic and diagnostic).

### BACKGROUND OF THE DISCLOSURE

Gene editing technology has emerged as a powerful and versatile technology with a potential for broad clinical applicability. In particular, the CRISPR-Cas system has been used by researchers to successfully disable or repair genes in a variety of cell types and species. Despite such advancements, off-target events remain a major issue and has prevented the broad applicability of the CRISPR-Cas system across targets and disease states. See, e.g., Jinek et al., Science 337:816-821 (2012); and Fu et al., Nature Biotechnology 31: 822-826 (2013).

Through the use of protein engineering, the specificity of the traditional Cas proteins (e.g., Streptococcus pyogenes Cas9 (SpCas9)) has been improved. For instance, compared to the wild-type SpCas9 protein, the eSpCas and HF-SpCas9 proteins offer much greater specificity. See, e.g., Slaymaker et al., Science 351: 84-88 (2016); and Kleinstiver et al., Nature 529: 490-495 (2016). However, off-target effects are still observed with significant frequency, particularly where single base mismatches are present during the gene editing process. In particular, when using CRISPR for in vitro cleavage, there is no repair mechanism unlike in cells, so DNA once damaged is not repaired, and there is no exception even in mismatch breakage. Therefore, under in vitro cleavage conditions, mismatch cleavage becomes more pronounced and the limitations of the present CRISPR-Cas systems become more apparent.

Accordingly, there remains a need for improved Cas9 proteins that allow for greater specificity and reduced off-target effects during gene editing.

### BRIEF SUMMARY OF THE DISCLOSURE

Provided herein is a Cas9 protein comprising a cavity domain that comprises a plurality of positively-charged amino acids, wherein at least one of the plurality of positively charged amino acids is modified (“amino acid modification”) compared to a corresponding wild-type Cas9 protein, and wherein the amino acid modification is capable of increasing the specificity of the Cas9 protein.

In some aspects, the plurality of positively-charged amino acids of the cavity domain comprises two, three, four, five, six, seven, eight, nine, 10, 11, or more amino acid modifications. In some aspects, the Cas9 protein comprises the amino acid sequence of SEQ ID NO: 1, and wherein the amino acid modification is at one or more of the following residues of SEQ ID NO: 1: R785, K789, R455, R721, R919, R1241, R939, K1189, K941, R1226, K1228, or a combination thereof. In some aspects, the amino acid modification is at residue R785, K1189, R1241, or a combination thereof. In some aspects, the amino acid modification is at residue K1189 and R1241. In some aspects, the amino acid modification is at residues R785, K1189, and R1241.

Provided herein is a Cas9 protein comprising the amino acid sequence set forth in SEQ ID NO: 1 with at least one amino acid modification, wherein the at least one amino acid modification is at residue K405, R455, K546, K561, K562, K564, K566, K578, K579, R618, R622, K664, R721, R785, K786, K788, K789, R807, K808, R849, R856, K914, K917, R919, R920, K921, K922, R926, K934, K936, R939, K941, K945, R1047, R1131, R1137, K1142, K1152, K1155, R1178, K1189, K1198, K1206, K1213, K1223, R1226, K1227, K1228, R1241, or a combination thereof, of SEQ ID NO: 1.

In some aspects, the at least one amino acid modification is at residue K405, R455, K566, K578, K664, R721, R785, K786, K789, K914, K917, R919, K921, K922, R926, K934, K936, R939, K941, K945, R1137, K1142, K1152, K1189, K1198, K1206, K1223, R1226, K1227, K1228, R1241, or a combination thereof, of SEQ ID NO: 1. In some aspects, the at least one amino acid modification is at residue R455, R785, R721, K789, R919, R1241, R939, K941, K1189, R1226, K1228, or a combination thereof, of SEQ ID NO: 1. In some aspects, the amino acid modification is at residues K1189 or R1241 of SEQ ID NO: 1. In some aspects, the amino acid modification is at residues: (i) K1189 and R1241 of SEQ ID NO: 1; (ii) R721 and R1241 of SEQ ID NO: 1; or (iii) R785 and R1241 of SEQ ID NO: 1. In some aspects, the amino acid modification is at residues: (i) R785, K1189, and R1241 of SEQ ID NO: 1; (ii) R721, K1189, and R1241 of SEQ ID NO: 1; or (iii) K1189, K1228, and R1241 of SEQ ID NO: 1.

For any of the Cas9 proteins described herein comprising at least one amino acid modification, in some aspects, the amino acid modification comprises an alanine substitution.

Present disclosure provides a Cas9 protein comprising, consisting of, or consisting essentially of the amino acid sequence set forth in SEQ ID NO: 2. Also provided herein is a Cas9 protein comprising, consisting of, or consisting essentially of the amino acid sequence set forth in SEQ ID NO: 3. Also provided herein is a Cas9 protein comprising, consisting of, or consisting essentially of the amino acid sequence set forth in SEQ ID NO: 4. Also provided herein is a Cas9 protein comprising, consisting of, or consisting essentially of the amino acid sequence set forth in SEQ ID NO: 5. Provided herein is a Cas9 protein comprising, consisting of, or consisting essentially of the amino acid sequence set forth in SEQ ID NO: 6. Provided herein is a Cas9 protein comprising, consisting of, or consisting essentially of the amino acid sequence set forth in SEQ ID NO: 7. Provided herein is a Cas9 protein comprising, consisting of, or consisting essentially of the amino acid sequence set forth in SEQ ID NO: 8. Provided herein is a Cas9 protein comprising, consisting of, or consisting essentially of the amino acid sequence set forth in SEQ ID NO: 9.

The present disclosure further provides a composition comprising any of the Cas9 proteins of the present disclosure. In some aspects, the composition further comprises a guide polynucleotide. In some aspects, the guide polynucleotide comprises a single guide RNA (sgRNA).

Provided herein is an isolated polynucleotide encoding any of the Cas9 proteins of the present disclosure. Also provided herein is a vector comprising the isolated polynucleotide. Also provided herein is a cell comprising the vector.

Disclosed herein is also a kit comprising any of the Cas9 proteins of the present disclosure, and instructions for use. In some aspects, the kit further comprises a guide polynucleotide. In some aspects, the guide polynucleotide comprises a single guide RNA (sgRNA).

Present disclosure provides a method of enriching for a first nucleotide sequence in a biological sample, which comprises the first nucleotide sequence and a second nucleotide sequence, the method comprising contacting the biological sample with any of the Cas9 proteins of the present disclosure, wherein the first nucleotide sequence comprises a mutation and the second nucleotide sequence does not comprise the mutation, and wherein the Cas9 protein is capable of recognizing the mutation and thereby cleave the second nucleotide sequence but not the first nucleotide sequence.

In some aspects, after the contacting the percentage of the first nucleotide sequence present in the biological sample is increased by at least about 1-fold, at least about 2-fold, at least about 3-fold, at least about 4-fold, at least about 5-fold, at least about 10-fold, at least about 15-fold, least about 20-fold, at least about 25-fold, at least about 30-fold, at least about 40-fold, or at least about 50-fold compared to the percentage of the first nucleotide sequence present in a reference sample (e.g., the biological sample prior to the contacting). In some aspects, after the contacting the amount of the second nucleotide sequence present in the biological sample is reduced by at least about 5%, at least about 10%, at least about 20%, at least about 30%, at least about 40%, at least about 50%, at least about 60%, at least about 70%, at least about 80%, at least about 90%, or at least about 100% compared to the amount of second nucleotide sequence present in a reference sample (e.g., the biological sample prior to the contacting). In some aspects, after the contacting at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of nucleotide molecules present in the biological sample comprise the first nucleotide sequence.

Also provided herein is a method of measuring the amount of a first nucleotide sequence, which comprises a mutation, in a biological sample, the method comprising contacting the biological sample with any of the Cas9 proteins of the present disclosure, wherein the contacting reduces the amount of a second nucleotide sequence present in the biological sample, and wherein the second nucleotide sequence does not comprise the mutation.

In some aspects, the first nucleotide sequence and the second nucleotide sequence are the same except for the mutation. In some aspects, the mutation comprises a substitution, insertion, deletion, deletion-insertion (indel), duplication, inversion, large genomic rearrangement, or a combination thereof.

In some aspects, the first nucleotide sequence comprises a single mutation. In some aspects, the first nucleotide sequence comprises multiple mutations. Where multiple mutations are present, in some aspects, each of the multiple mutations is the same. In some aspects, two or more of the multiple mutations are different.

In some aspects, the mutation is within: (i) a target site to which a guide polynucleotide binds, (ii) a protospacer adjacent motif (PAM), or (iii) both (i) and (ii).

In some aspects, the biological sample was obtained from a subject suffering from or having an increased risk of developing a disease. In some aspects, the mutation is associated with the disease.

In some aspects, the first nucleotide sequence comprises a circulating tumor DNA (ctDNA) and wherein the second nucleotide sequence comprises a non-ctDNA.

Present disclosure further provides a method of diagnosing a disease in a subject in need thereof, the method comprising detecting whether the amount of a nucleotide sequence, which comprises a mutation that is associated with the disease, is increased in a biological sample obtained from the subject compared to a corresponding amount present in a reference sample (e.g., biological sample obtained from a subject who does not suffer from the disease), wherein prior to the detecting, the biological sample was contacted with any of the Cas9 proteins described herein.

In some aspects, the subject has or is at risk of developing the disease if the amount of the nucleotide sequence, which comprises the mutation, is increased in the biological sample compared to the corresponding amount present in the reference sample. In some aspects, the amount of the nucleotide sequence, which comprises the mutation, is increased in the biological sample by at least about 1-fold, at least about 5-fold, at least about 10-fold, at least about 15-fold, at least about 20-fold, at least about 25-fold, at least about 30-fold, at least about 40-fold, or at least about 50-fold compared to the corresponding amount present in the reference sample.

In some aspects, the diagnosing is performed ex vivo.

In some aspects, the disease comprises a cancer, hematologic disease, neurodegenerative/neurologic disease, infectious disease, rheumatoid disease, allergic disease, psychiatric disease, optical disease, endocrinologic disease, congenital disease, cardiovascular disease, pulmonary disease, nephrologic disease, gastrologic disease, hepatologic disease, or a combination thereof. In some aspects, the cancer comprises a lung cancer (e.g., non-small cell lung cancer), breast cancer, pancreatic cancer, biliary duct cancer, gallbladder cancer, liver cancer, colorectal cancer, kidney cancer, prostate cancer, gastric cancer, ovarian cancer, uterine cancer, cervical cancer, muscular skeletal cancer, or a combination thereof. In some aspects, the neurodegenerative/neurologic disease comprises an Alzheimer's disease, Parkinson's disease, amyotrophic lateral sclerosis, Friedreich's Ataxia, Huntington's disease, Lew body disease, spinal muscular atrophy, stroke, or a combination thereof.

Provided herein is a method of reducing an occurrence of an off-target cleavage of a nucleic acid sequence during a CRISPR-based gene editing, comprising contacting the nucleic acid sequence with a complex comprising a Cas9 protein and a guide polynucleotide, wherein the Cas9 protein comprises an amino acid modification which is capable of increasing the specificity of the Cas9 protein, thereby reducing the occurrence of an off-target cleavage. In some aspects, the Cas9 protein comprises any of the Cas9 proteins of the present disclosure.

In some aspects, the occurrence of an off-target cleavage is reduced by at least about 5%, at least about 10%, at least about 20%, at least about 30%, at least about 40%, at least about 50%, at least about 60%, at least about 70%, at least about 80%, at least about 90%, or at least about 100% compared to the occurrence of an off-target cleavage with a reference Cas9 protein. In some aspects, the reference Cas9 protein comprises a corresponding Cas9 protein that does not comprise the amino acid modification. In some aspects, the reference Cas9 protein comprises the amino acid sequence set forth in any one of SEQ ID NO: 244, SEQ ID NO: 1, SEQ ID NO: 245, SEQ ID NO: 246, SEQ ID NO: 247, or SEQ ID NO: 248.

Disclosed herein is a method of increasing a specificity of a Cas9 protein comprising modifying at least one amino acid residue of the Cas9 protein, wherein the at least one amino acid residue is capable of interacting with a backbone phosphate of a DNA sequence.

In some aspects, the at least one amino acid residue which is modified comprises residue K405, R455, K546, K561, K562, K564, K566, K578, K579, R618, R622, K664, R721, R785, K786, K788, K789, R807, K808, R849, R856, K914, K917, R919, R920, K921, K922, R926, K934, K936, R939, K941, K945, R1047, R1131, R1137, K1142, K1152, K1155, R1178, K1189, K1198, K1206, K1213, K1223, R1226, K1227, K1228, R1241, or a combination thereof, corresponding to the amino acid sequence set forth in SEQ ID NO: 1. In some aspects, the at least one amino acid residue which is modified comprises residue K405, R455, K566, K578, K664, R721, R785, K786, K789, K914, K917, R919, K921, K922, R926, K934, K936, R939, K941, K945, R1137, K1142, K1152, K1189, K1198, K1206, K1223, R1226, K1227, K1228, R1241, or a combination thereof, corresponding to the amino acid sequence set forth in SEQ ID NO: 1. In some aspects, the at least one amino acid residue which is modified comprises residue R455, R785, R721, K789, R919, R1241, R939, K941, K1189, R1226, K1228, or a combination thereof, corresponding to the amino acid sequence set forth in SEQ ID NO: 1. In some aspects, the at least one amino acid residue which is modified comprises K1189 or R1241, corresponding to SEQ ID NO: 1. In some aspects, the at least one amino acid residue which is modified comprises: (i) K1189 and R1241; (ii) R721 and R1241; (iii) R785 and R1241; or (iv) K1189 and R1241, corresponding to SEQ ID NO: 1. In some aspects, the at least one amino acid residue which is modified comprises: (i) R785, K1189, and R1241; (ii) R721, K1189, and R1241; or (iii) K1189, K1228, and R1241, corresponding to SEQ ID NO: 1.

In some aspects, the amino acid modification comprises an alanine substitution.

In some aspects, after the modification, the selectivity of the Cas9 protein is increased by at least about 1-fold, at least about 5-fold, at least about 10-fold, at least about 15-fold, at least about 20-fold, at least about 25-fold, at least about 30-fold, at least about 40-fold, or at least about 50-fold compared to the specificity of a reference Cas9 protein. In some aspects, the reference Cas9 protein comprises a corresponding Cas9 protein that does not comprise the amino acid modification. In some aspects, the reference Cas9 protein comprises the amino acid sequence set forth in any one of SEQ ID NO: 244, SEQ ID NO: 1, SEQ ID NO: 245, SEQ ID NO: 246, SEQ ID NO: 247, or SEQ ID NO: 248.

In some aspects, after the modification the Cas9 protein is capable of differentiating between a first nucleotide sequence comprising a mutation and a second nucleotide sequence that does not comprise the mutation, such that the Cas9 protein cleaves the second nucleotide sequence but not the first nucleotide sequence. In some aspects, the mutation is within: (i) a target site to which a guide polynucleotide binds, (ii) a protospacer adjacent motif (PAM), or (iii) both.

Provided herein is a method of genetically modifying a cell, comprising contacting a cell with any of the Cas9 proteins of the present disclosure, wherein the contacting results in the modification of one or more DNA sequences of the cell. In some aspects, the cell comprises an eukaryotic cell, a yeast cell, a plant cell, a mammalian cell, or a combination thereof.

### BRIEF DESCRIPTION OF THE FIGURES

FIGS. 1A, 1B, 1C, and 1D provide comparison of the specificity of wild-type SpCas9 and FnCas9 proteins with single-base mismatched sgRNA against KRAS target sequence. As further described in Example 1, sgRNAs with single-base mismatch at different positions within the target sequence were constructed, and the specificity of the Cas9 proteins was determined by measuring their ability to cleave the target KRAS sequence with the different sgRNAs using an in vitro cleavage assay. Table 5 provides the sequences for the different KRAS-targeting sgRNAs tested (see Experiment labeled “Specificity comparison_SpCas9 versus FnCas9”). FIG. 1A provides the results for the wild-type SpCas9 protein with the control KRAS sgRNA (no mismatch with target; “T”) (i.e., “KRAS-T” in Table 5) or any one of the following KRAS sgRNAs: KRAS-1 to KRAS-10 as identified in Table 5. FIG. 1B provides the results for the wild-type SpCas9 protein with any one of the following KRAS sgRNAs: KRAS-11 to KRAS-20 as identified in Table 5. FIG. 1C provides the results for the wild-type FnCas9 protein with the control KRAS sgRNA (no mismatch with target; “T”) (“KRAS-T” in Table 5) or any one of KRAS-1 to KRAS-10 sgRNAs. FIG. 1D provides the results for the wild-type FnCas9 protein with any one of KRAS-11 to KRAS-20 sgRNAs.

FIGS. 2A, 2B, 2C, and 2D provide comparison of the specificity of the following SpCas9 protein variants with single-base mismatched sgRNA against KRAS target sequence. FIG. 2A provides the results for the SpCas9-HF1 protein. FIG. 2B provides the results for the SpCas9-HF4 protein. FIG. 2C provides the results for the eSpCas9(1.0) protein. FIG. 2D provides the results for the eSpCas9(1.1) protein. In each of FIGS. 2A-2D, the results provided to the left are with the control KRAS sgRNA (no mismatch with target; “T”) (“KRAS-T” in Table 5) or any one of KRAS-1 to KRAS-10 sgRNAs (as identified in Table 5); and the results to the right are with any one of KRAS-11 to KRAS-20 sgRNAs (as identified in Table 5). The KRAS sgRNAs are the same as those described in FIGS. 1A-1D.

FIG. 3 provides a heat map showing a quantitative comparison of the cleavage efficiency data provided in FIGS. 1A-1D and 2A-2D.

FIGS. 4A and 4B show the specificity of different FnCas9 protein variants with single-base mismatched sgRNA against the KRAS target sequence. FIG. 4A provides a heat map comparing the cleavage efficiency of the different FnCas9 protein variants. To generate the different FnCas9 protein variants shown, an alanine substitution was made at one of the following residues within the wild-type FnCas9 protein: K405, R455, K546, K561, K562, K564, K566, K578, K579, R618, R622, K664, R721, R785, K786, K788, K789, R807, K808, R849, R856, K914, K917, R919, R920, K921, K922, R926, K934, K936, R939, K941, K945, R1047, R1131, R1137, K1142, K1152, K1155, R1178, K1189, K1198, K1206, K1213, K1223, R1226, K1227, K1228, or R1241 (see along the top of the heat map). The wild-type FnCas9 protein was used as a control (WT). The KRAS sgRNAs used to generate the data are shown to the left of the heat map and correspond to those described in FIG. 1A (i.e., control KRAS-T and KRAS-1 to KRAS-10 sgRNAs). FIG. 4B provides a bar graph comparison of the specificity score of the different FnCas9 protein variants. The specificity score was calculated as the squared difference between the cleavage rate of the on-target and average of off-targets. The specificity score shown was normalized to the specificity score of the wild-type FnCas9 protein (i.e., specificity score=1). The horizontal line represents a specificity score of 1. The specific amino acid substitutions are shown along the bottom of the bar graph.

FIG. 5 provides a crystal structure of the FnCas9 protein. Exemplary amino acid residues that can be modified to increase the specificity of the FnCas9 protein are identified.

FIG. 6 shows the ability of the following FnCas9 protein variants to cleave target NRAS gene sequences with single-base mismatched sgRNA: (i) FnCas9 protein with a single modification: at residue K1189 (FnCas9-K1189A); (ii) FnCas9 protein with a single modification: at residue R1241 (FnCas9-R1241A); (iii) FnCas9 protein with double modifications: at residues K1189 and R1241 (FnCas9-K1189A, R1241A; also referred to herein as “FnCas9-AF1”); and (iv) FnCas9 protein with triple modifications: at residues R785, K1189, and R1241 (FnCas9-R785A, K1189A, R1241A; also referred to herein as “FnCas9-AF2”). Table 5 provides the sequences for the different NRAS-targeting sgRNAs tested. Nucleotides shown to the left of each heat map (i.e., G, C, A, U) correspond to the specific single-base mismatch that was made to the sgRNA tested.

FIGS. 7A and 7B provide heat map analysis of the cleavage efficiency of additional FnCas9 protein variants comprising single, double, or triple mutations with single-base mismatched sgRNA against KRAS target sequence (FIG. 7A) or NRAS target sequence (FIG. 7B). In each of the figures, the table provided above the heat map shows the different amino acid modifications that were made to generate the different FnCas9 protein variants tested. The sequences for the different sgRNAs used are provided to the left of each heat map. In FIG. 7A, the KRAS sgRNAs are as follows (from top to bottom): KRAS-WT (SEQ ID NO: 48), KRAS-1 (SEQ ID NO: 49), KRAS-2 (SEQ ID NO: 50), KRAS-3 (SEQ ID NO: 51), KRAS-4 (SEQ ID NO: 52), KRAS-5 (SEQ ID NO: 53), KRAS-6 (SEQ ID NO: 54), KRAS-7 (SEQ ID NO: 55), KRAS-8 (SEQ ID NO: 56), KRAS-9 (SEQ ID NO: 57), KRAS-10 (SEQ ID NO: 58), KRAS-11 (SEQ ID NO: 59), KRAS-12 (SEQ ID NO: 60), KRAS-13 (SEQ ID NO: 61), KRAS-14 (SEQ ID NO: 62), KRAS-15 (SEQ ID NO: 63), KRAS-16 (SEQ ID NO: 64), KRAS-17 (SEQ ID NO: 65), KRAS-18 (SEQ ID NO: 66), KRAS-19 (SEQ ID NO: 67), and KRAS-20 (SEQ ID NO: 68). In FIG. 7B, the NRAS sgRNAs are as follows (from top to bottom): NRAS-WT (SEQ ID NO: 105), NRAS-1-G (SEQ ID NO: 106), NRAS-2-C(SEQ ID NO: 109), NRAS-3-T (SEQ ID NO: 112), NRAS-4-G (SEQ ID NO: 115), NRAS-5-T (SEQ ID NO: 118), NRAS-6-A (SEQ ID NO: 121), NRAS-7-T (SEQ ID NO: 124), NRAS-8-C(SEQ ID NO: 127), NRAS-9-C(SEQ ID NO: 130), NRAS-10-A (SEQ ID NO: 133), NRAS-11-G (SEQ ID NO: 136), NRAS-12-T (SEQ ID NO: 139), NRAS-13-A (SEQ ID NO: 142), NRAS-14-T (SEQ ID NO: 145), NRAS-15-G (SEQ ID NO: 148), NRAS-16-T (SEQ ID NO: 151), NRAS-17-C(SEQ ID NO: 154), NRAS-18-C(SEQ ID NO: 157), NRAS-19-A (SEQ ID NO: 160), and NRAS-20-A (SEQ ID NO: 163).

FIGS. 8A, 8B, and 8C provide comparison of the specificity of the wild-type FnCas9 and FnCas9-AF2 proteins with single-base mismatched sgRNA against KRAS and EGFR target sequences. FIG. 8A provides the KRAS (top) (GTAGTTGGAGCTGGTGGCGT; SEQ ID NO: 249) and EGFR (bottom) (CAGATTTTGGGCTGGCCAAA; SEQ ID NO: 250) target sequences. FIG. 8B shows the cleavage efficiency data for the wild-type FnCas9 against the KRAS (top heat map) and EGFR (bottom heat map) sequences. FIG. 8C shows the cleavage efficiency data for the FnCas9-AF2 against the KRAS (top heat map) and EGFR (bottom heat map) sequences. Table 5 provides the sequences for the different KRAS-targeting and EGFR-targeting sgRNAs tested. The nucleotides shown to the left of each heat map (i.e., G, C, A, U) correspond to the specific single-base mismatch that was made to the sgRNA tested.

FIGS. 9A and 9B provide digested genome sequencing (Digenome-seq) analysis comparing the unbiased genome-wide off-target occurrences observed after digestion of genomic DNA of HEK293T cells with several Cas9 protein variants FIG. 9A provides Manhattan plots showing the DNA cleavage position generated by the different Cas9 proteins: (i) wild-type SpCas9 protein (top left plot); (ii) wild-type FnCas9 protein (top right plot); (iii) eSpCas9(1.1) (middle left plot); (iv) FnCas9-AF1 (middle right plot); (v) SpCas9-H4 (bottom left plot); and (vi) FnCas9-AF2 (bottom right plot). FIG. 9B provides Venn diagrams showing the number of off-target generated by SpCas9-WT, SpCas9-HF4, FnCas9-WT, FnCas9-AF2 (left diagram) and by eSpCas9(1.1), SpCas9-HF4, FnCas9-AF1, FnCas9-AF2 (right diagram).

FIG. 10 provides a schematic illustrating the use of a Cas9 protein with advanced specificity (e.g., described herein) to enrich for mutant DNA present in a sample comprising cell-free DNA (cfDNA). Specifically, the sgRNA is designed to cleave the wild-type DNA (major allele DNA in cfDNA; “wtDNA”) while the mutant DNA (minor allele DNA in cfDNA; “mtDNA”) remains uncleaved, resulting in the subsequent enrichment of the mutant DNA. As shown, mutant DNA can be categorized into one of two types based on the position of the mutation present: (1) type I-mutation(s) in the PAM site; and (2) type II-mutation(s) within the sgRNA target sequence but outside of the PAM site. CRISPR-Cas proteins known in the art can generally recognize mutations within the PAM site. As a result, such Cas proteins can effectively distinguish type I mtDNA from wtDNA but not type II mtDNA, resulting in the cleavage of both the wtDNA and the type II mtDNA (see illustration provided below the dashed line). In contrast, the Cas9 proteins described herein (having advanced specificity) can effectively distinguish both type I and type II mtDNAs, resulting in the cleavage of only the wtDNA.

FIG. 11 provides the ratio of applicable target mutants that are observed to occur either within a PAM region (type I mutants; light gray bar) or outside a PAM region (type II mutants; dark gray bar) for various cancer types and targetable by CRISPR enrichment. The cancer types shown include: lung cancer, breast cancer, liver cancer, pancreatic cancer, and thyroid cancer.

FIGS. 12A, 12B, 12C, and 12D show the enrichment for type II EGFR or KRAS mutant DNA sequences after digestion of a mixture comprising mutant and wild-type DNA sequences with the FnCas9-AF2 protein. Enrichment is shown as the ratio of mutant to wild-type DNA sequences observed after the digestion (enriched mutant ratio). The mutated EGFR sequence had one of the following mutations within the sgRNA target sequence but outside of the PAM site: T790M (FIG. 12A), L858R (FIG. 12B), or exon19 deletion (FIG. 12C). The mutated KRAS sequence had the G12D mutation (FIG. 12D), which was positioned out of the PAM site but within the sgRNA target sequence. Prior to digestion, the mixture contained 5%, 1%, 0.1%, or 0% mutant DNA. Samples that were not digested with any Cas9 protein were used as control (“Cas9 (−)”).

FIG. 13 provides heat map analysis showing CRISPR-Cas9-mediated enrichment of mutant DNA sequences in human cancer patient samples. Blood and tissue samples from 10 non-small cell lung cancer patients (identified as P03, P07-P12, P18, P22, and P26) with 9 in stage I and 1 in stage II were acquired and analyzed for the presence of 1,056 genomic variants. The results are shown as follows: (1) non-enriched tissue sample (i.e., original tissue; “OT”); (2) tissue samples enriched with Cas9 protein with advanced specificity (i.e., CRISPR-enriched tissue; “CT”); (3) non-enriched blood sample (i.e., original cfDNA; “Oc”); and (4) blood sample enriched with Cas9 protein with advanced specificity (i.e., CRISPR-enriched cfDNA; “Cc”).

FIGS. 14A, 14B, 14C, and 14D provide comparison of the specificity of the wild-type FnCas9 and FnCas9-AF2 proteins with single-base mismatched sgRNA against NRAS target sequence. Table 5 provides the sequences for the different NRAS-targeting sgRNAs tested (NRAS-sgRNA #1-#20). FIGS. 14A and 14C provide the results for the wild-type SpCas9 protein and FnCas9-AF2 protein, respectively, with the following NRAS sgRNAs (as identified in Table 5): (T) NRAS-WT (no mismatch to target), (1) NRAS-1-G, (2) NRAS-2-C, (3) NRAS-3-T, (4) NRAS-4-G, (5) NRAS-5-T, (6) NRAS-6-A, (7) NRAS-7-T, (8) NRAS-8-C, (9) NRAS-9-C, and (10) NRAS-10-A. FIG. 14B and FIG. 14D provide the results for the wild-type SpCas9 protein and FnCas9-AF2 protein, respectively, with the following NRAS sgRNAs (as identified in Table 5): (11) NRAS-11-G, (12) NRAS-12-T, (13) NRAS-13-A, (14) NRAS-14-T, (15) NRAS-15-G, (16) NRAS-16-T, (17) NRAS-17-C, (18) NRAS-18-C, (19) NRAS-19-A, and (20) NRAS-20-A.

FIG. 15 provides heat map analysis showing CRISPR-Cas9-mediated enrichment of 392 genomic variants that satisfy the criteria set forth in Categories 1, 3, 4, and 6 (as described in Table 6) in non-small cell lung cancer (NSCLC) patient samples (as described in FIG. 13). The subjects were sorted in the order of patient number in the four groups, and there were 10 subjects in each group. Original tissue (OT), CRISPR enriched tissue (CT), original cfDNA (Oc), and CRISPR enriched cfDNA (Cc) groups were arranged in order. Statistical significance was presented between the OT and CT groups (tissue correlation; Tr) and between the Oc and Cc groups (cfDNA correlation; cr). The higher the fold change (FC) value, the higher the value in the CRISPR enriched (CE) group, and the darker the p-value (PV) value treated with −log 10, the higher the statistical significance.

FIGS. 16A and 16B provide heat map analysis showing CRISPR-Cas9-mediated enrichment of a particular subset of the 392 genomic variants described in FIG. 15 based on statistical significance. Higher the fold change (FC) value, higher the value in the CRISPR enriched (CE) group, and darker the p-value (PV) value treated with −log 10, higher the statistical significance. FIG. 16A provides results for 11 variants with p-value<0.05 and FC>0.1 as compared between OT and CT (tissue correlation; Tr). FIG. 16B provides results for 17 variants with p-value<0.05 and FC>0.1 as compared between Oc and Cc (cfDNA correlation; cr). In FIGS. 16A and 16B, the different variants are provided immediately to the right of the heat maps.

FIGS. 17A, 17B, 17C, and 17D provide boxplots of the heat map analysis provided in FIGS. 13, 15, 16A, and 16B, respectively. Specifically, the boxplots show the statistical significance in the detection of the genomic variants among the different NSCLC patient samples. The subjects were sorted in the order of patient number in the four groups, and there were 10 subjects in each group. Original tissue (OT), original cfDNA (Oc), CRISPR enriched (CE) tissue (CT), and CE cfDNA (Cc) groups were arranged in order. In each group, statistical groups and their p-values were indicated. Kruskal wallis tests were performed by four groups, and statistical significant p-values (<2.2e-16) were detected. (FIG. 17A) Total landscape, for 1,056 variants. In each of the groups, 1,056*10=10,560 plots were listed. (FIG. 17B) After filtered by conditions, total 392 variants and 392*10=3,920 plots were used to make boxplot. In the comparisons of tissues (FIG. 17C), and cfDNAs (FIG. 17D), total 11 and 17 variants of 180 and 276 plots were used to boxplot, respectively.

FIGS. 18A, 18B, 18C, 18D, 18E, 18F, 18G, 18H, 18I, 18J, 18K, 18L, 18M, 18N, 18O, and 18P provide correlation plots of the total genomic variants detected before and after CRISPR-Cas9-mediated enrichment (CE) in the different NSCLC patient samples: original tissues (OT), original cfDNA (Oc), CE tissues (CT), and CE cfDNA (Cc). For all figures shown, the black dots represent the higher ratio when comparing before and after enrichment. In FIGS. 18A, 18E, 18I, and 18M, OT and Oc samples are compared prior to the enrichment. In FIGS. 18B, 18F, 18J, and 18N, OT and CT samples are compared after the enrichment. In FIGS. 18C, 18G, 18K, and 18O, Oc and Cc groups are compared in the cfDNA samples. In FIGS. 18D, 18H, 18L, and 18P, CT and Cc groups are compared to confirm the CE patters observed in the cfDNA samples. Additionally, in FIGS. 18A, 18B, 18C, 18D, 18E, 18F, 18G, and 18H, correlation plots of all genomic variants observed and filtered variants are provided. In FIGS. 18I, 18J, 18K, 18L, 18M, 18N, 18O, and 18P, correlation plots of filtered by the groups between before- and after-CE in tissues and cfDNAs are provided.

FIG. 19 provides crystal structure of the FnCas9 protein in which amino acids that are capable of interacting with the phosphate backbone of either the target or non-target DNA strands are shown. The specific amino acids (49 total) are identified in FIG. 4B.

### DETAILED DESCRIPTION OF THE DISCLOSURE

The present disclosure is directed to Cas9 proteins that have been modified to comprise one or more features that render them distinct (e.g., structurally and/or functionally) from a reference Cas9 protein known in the art (e.g., wild-type S. pyogenes Cas9 protein). For example, as further described herein, the Cas9 proteins of the present disclosure comprise one or more amino acid modifications, which enhance the specificity of the Cas9 proteins. Accordingly, compared to a reference Cas9 protein, the Cas9 proteins described herein can more accurately recognize and distinguish base mismatches within a target gene sequence. As demonstrated herein, because of such enhanced specificity, in some aspects, a Cas9 protein described herein is associated with much reduced off-target effects (e.g., off-target binding, editing, and/or cleavage activity). In some aspects, the Cas9 proteins described herein are associated with increased on-target effects (e.g., on-target binding, editing, and/or cleavage activity). In some aspects, a Cas9 protein described herein is associated with both reduced off-target effects and increased on-target effects. Additional aspects of the present disclosure are provided throughout the present application.

Before the present disclosure is described in greater detail, it is to be understood that this disclosure is not limited to the particular compositions or process steps described, which can, of course, vary. As will be apparent to those of skill in the art upon reading this disclosure, each of the individual aspects described and illustrated herein has discrete components and features which can be readily separated from, or combined with, the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “a” or “an” entity refers to one or more of that entity; for example, “a Cas9 protein” is understood to represent one or more Cas9 proteins. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2nd ed., 2008, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited (e.g., 10), it is to be understood that values that are about the same quantity or amount as the recited value (e.g., ±10%) are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil. It is to be understood that in the disclosed sequences, T and U are interchangeable depending on whether the sequence is a DNA or an RNA. For example, target sequences are presented as DNAs (A/T/C/G) in the present disclosure, whereas the guide RNAs are presented as RNAs (A/U/C/G).

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

As used herein, the term “Cas9 protein” (including any variants thereof) refers to a polypeptide that can interact with a guide RNA (gRNA) molecule and, in concert with the gRNA molecule, localize to a site comprising a target sequence and, in some aspects, a PAM. As further described elsewhere in the present disclosure, Cas9 proteins described herein have been modified, altered, or engineered to comprise one or more properties (e.g., enhanced specificity). Accordingly, the terms “Cas9 protein described herein,” “Cas9 protein provided herein,” “Cas9 protein of the present disclosure” (including variants thereof) refer to Cas9 proteins that comprise one or more of the amino acid modifications described herein and exhibits increased specificity as compared to a reference Cas9 protein (e.g., wild-type Cas9 protein). Additionally, unless indicated otherwise, the terms “altered,” “engineered,” and “modified” are interchangeable and, as used in this context, refer merely to a difference from a reference or naturally occurring sequence, and impose no specific process or origin limitations. Additional disclosures relating to the Cas9 proteins of the present disclosure are provided elsewhere herein.

As described herein, in some aspects, a Cas9 protein that can be modified using the disclosures provided herein comprises Cas9 protein from Francisella novicida (“FnCas9”). The amino acid sequence for the wild-type FnCas9 protein is provided in Table 1 (below) (i.e., SEQ ID NO: 1). Accordingly, in some aspects, the Cas9 proteins provided herein comprise an amino acid sequence that differs from SEQ ID NO: 1 by one or more amino acids. In some aspects, the amino acid sequence of a Cas9 protein provided herein has less than about 99.999%, less than about 99.998%, less than about 99.997%, less than about 99.996%, less than about 99.995%, less than about 99.994%, less than about 99.993%, less than about 99.992%, less than about 99.991%, less than about 99.99%, less than about 99.8%, less than about 99.7%, less than about 99.6%, less than about 99.5%, less than about 99.4%, less than about 99.3%, less than about 99.2%, less than about 99.1%, less than about 99%, less than about 98%, less than about 97%, less than about 96%, or less than about 95% sequence identity to the amino acid sequence set forth in SEQ ID NO: 1. It will be apparent to those skilled in the arts that the disclosures provided herein can be applicable to any suitable Cas9 proteins known in the art (wild-type and variants thereof). Non-limiting examples of such Cas9 proteins are described in, e.g., U.S. Publication No. 2018/0051281 A1, which is incorporated herein by reference in its entirety.

In some aspects, a Cas9 protein that can be modified comprises Cas9 protein from Streptococcus pyogenes (“SpCas9”). The amino acid sequence for the wild-type SpCas9 protein is provided in Table 1 (below) (i.e., SEQ ID NO: 244). Accordingly, in some aspects, the Cas9 proteins provided herein comprise an amino acid sequence that differs from SEQ ID NO: 244 by one or more amino acids. In some aspects, the amino acid sequence of a Cas9 protein provided herein has less than about 99.999%, less than about 99.998%, less than about 99.997%, less than about 99.996%, less than about 99.995%, less than about 99.994%, less than about 99.993%, less than about 99.992%, less than about 99.991%, less than about 99.99%, less than about 99.8%, less than about 99.7%, less than about 99.6%, less than about 99.5%, less than about 99.4%, less than about 99.3%, less than about 99.2%, less than about 99.1%, less than about 99%, less than about 98%, less than about 97%, less than about 96%, or less than about 95% sequence identity to the amino acid sequence set forth in SEQ ID NO: 244.

Additional examples of Cas9 proteins that are suitable for the present disclosure are provided elsewhere in the present disclosure.

TABLE 1
Amino Acid Sequence for Cas9 Proteins

| | |
|---|---|
| Wild-type FnCas9 Protein | MNFKILPIAIDLGVKNTGVFSAFYQKGTSLERLDNKNGKVYELSKDSYTLLMNNRT |
| (UniProt No. A0Q5Y3) | ARRHQRRGIDRKQLVKRLFKLIWTEQLNLEWDKDTQQAISFLFNRRGFSFITDGYS |
| (SEQ ID NO: 1) | PEYLNIVPEQVKAILMDIFDDYNGEDDLDSYLKLATEQESKISEIYNKLMQKILEF |
| | KLMKLCTDIKDDKVSTKTLKEITSYEFELLADYLANYSESLKTQKFSYTDKQGNLK |
| | ELSYYHHDKYNIQEFLKRHATINDRILDTLLTDDLDIWNFNFEKFDFDKNEEKLQN |
| | QEDKDHIQAHLHHFVFAVNKIKSEMASGGRHRSQYFQEITNVLDENNHQEGYLKNF |
| | CENLHNKKYSNLSVKNLVNLIGNLSNLELKPLRKYFNDKIHAKADHWDEQKFTETY |
| | CHWILGEWRVGVKDQDKKDGAKYSYKDLCNELKQKVTKAGLVDFLLELDPCRTIPP |
| | YLDNNNRKPPKCQSLILNPKFLDNQYPNWQQYLQELKKLQSIQNYLDSFETDLKVL |
| | KSSKDQPYFVEYKSSNQQIASGORDYKDLDARILQFIFDRVKASDELLLNEIYFQA |
| | KKLKQKASSELEKLESSKKLDEVIANSQLSQILKSQHTNGIFEQGTFLHLVCKYYK |
| | QRQRARDSRLYIMPEYRYDKKLHKYNNTGRFDDDNQLLTYCNHKPRQKRYQLLNDL |
| | AGVLQVSPNFLKDKIGSDDDLFISKWLVEHIRGFKKACEDSLKIQKDNRGLLNHKI |
| | NIARNTKGKCEKEIFNLICKIEGSEDKKGNYKHGLAYELGVLLFGEPNEASKPEFD |
| | RKIKKFNSIYSFAQIQQIAFAERKGNANTCAVCSADNAHRMQQIKITEPVEDNKDK |
| | IILSAKAQRLPAIPTRIVDGAVKKMATILAKNIVDDNWQNIKQVLSAKHQLHIPII |
| | TESNAFEFEPALADVKGKSLKDRRKKALERISPENIFKDKNNRIKEFAKGISAYSG |
| | ANLTDGDFDGAKEELDHIIPRSHKKYGTLNDEANLICVTRGDNKNKGNRIFCLRDL |
| | ADNYKLKQFETTDDLEIEKKIADTIWDANKKDFKFGNYRSFINLTPQEQKAFRHAL |
| | FLADENPIKQAVIRAINNRNRTFVNGTQRYFAEVLANNIYLRAKKENLNTDKISFD |
| | YFGIPTIGNGRGIAEIRQLYEKVDSDIQAYAKGDKPQASYSHLIDAMLAFCIAADE |
| | HRNDGSIGLEIDKNYSLYPLDKNTGEVFTKDIFSQIKITDNEFSDKKLVRKKAIEG |
| | FNTHRQMTRDGIYAENYLPILIHKELNEVRKGYTWKNSEEIKIFKGKKYDIQQLNN |
| | LVYCLKFVDKPISIDIQISTLEELRNILTTNNIAATAEYYYINLKTQKLHEYYIEN |
| | YNTALGYKKYSKEMEFLRSLAYRSERVKIKSIDDVKQVLDKDSNFIIGKITLPFKK |
| | EWQRLYREWQNTTIKDDYEFLKSFFNVKSITKLHKKVRKDFSLPISTNEGKFLVKR |
| | KTWDNNFIYQILNDSDSRADGTKPFIPAFDISKNEIVEAIIDSFTSKNIFWLPKNI |
| | ELQKVDNKNIFAIDTSKWFEVETPSDLRDIGIATIQYKIDNNSRPKVRVKLDYVID |
| | DDSKINYFMNHSLLKSRYPDKVLEILKOSTIIEFESSGFNKTIKEMLGMKLAGIYN |
| | ETSNN |
| Wild-Type SpCas9 | MDKKYSIGLDIGTNSVGWAVITDEYKVPSKKFKVLGNTDRHSIKKNLIGALLFDSG |
| Protein (UniProt No. | ETAEATRLKRTARRRYTRRKNRICYLQEIFSNEMAKVDDSFFHRLEESFLVEEDKK |
| Q99ZW2-1) (SEQ ID NO: | HERHPIFGNIVDEVAYHEKYPTIYHLRKKLVDSTDKADLRLIYLALAHMIKFRGHF |
| 244) | LIEGDLNPDNSDVDKLFIQLVQTYNQLFEENPINASGVDAKAILSARLSKSRRLEN |
| | LIAQLPGEKKNGLFGNLIALSLGLTPNFKSNFDLAEDAKLQLSKDTYDDDLDNLLA |
| | QIGDQYADLFLAAKNLSDAILLSDILRVNTEITKAPLSASMIKRYDEHHQDLTLLK |
| | ALVRQQLPEKYKEIFFDQSKNGYAGYIDGGASQEEFYKFIKPILEKMDGTEELLVK |
| | LNREDLLRKQRTFDNGSIPHQIHLGELHAILRRQEDFYPFLKDNREKIEKILTFRI |
| | PYYVGPLARGNSRFAWMTRKSEETITPWNFEEVVDKGASAQSFIERMTNFDKNLPN |
| | EKVLPKHSLLYEYFTVYNELTKVKYVTEGMRKPAFLSGEQKKAIVDLLFKTNRKVT |
| | VKQLKEDYFKKIECFDSVEISGVEDRFNASLGTYHDLLKIIKDKDFLDNEENEDIL |
| | EDIVLTLTLFEDREMIEERLKTYAHLFDDKVMKQLKRRRYTGWGRLSRKLINGIRD |
| | KQSGKTILDFLKSDGFANRNFMQLIHDDSLTFKEDIQKAQVSGQGDSLHEHIANLA |
| | GSPAIKKGILQTVKVVDELVKVMGRHKPENIVIEMARENQTTQKGQKNSRERMKRI |
| | EEGIKELGSQILKEHPVENTQLQNEKLYLYYLQNGRDMYVDQELDINRLSDYDVDH |
| | IVPQSFLKDDSIDNKVLTRSDKNRGKSDNVPSEEVVKKMKNYWRQLLNAKLITQRK |
| | FDNLTKAERGGLSELDKAGFIKRQLVETRQITKHVAQILDSRMNTKYDENDKLIRE |
| | VKVITLKSKLVSDFRKDFQFYKVREINNYHHAHDAYLNAVVGTALIKKYPKLESEF |
| | VYGDYKVYDVRKMIAKSEQEIGKATAKYFFYSNIMNFFKTEITLANGEIRKRPLIE |
| | TNGETGEIVWDKGRDFATVRKVLSMPQVNIVKKTEVQTGGFSKESILPKRNSDKLI |
| | ARKKDWDPKKYGGFDSPTVAYSVLVVAKVEKGKSKKLKSVKELLGITIMERSSFEK |
| | NPIDFLEAKGYKEVKKDLIIKLPKYSLFELENGRKRMLASAGELQKGNELALPSKY |
| | VNFLYLASHYEKLKGSPEDNEQKQLFVEQHKHYLDEITEQISEFSKRVILADANLD |
| | KVLSAYNKHRDKPIREQAENIIHLFTLTNLGAPAAFKYFDTTIDRKRYTSTKEVLD |
| | ATLIHQSITGLYETRIDLSQLGGD |

As used herein, the term “eSpCas9(1.1)” refers to a modified SpCas9 protein with the following amino acid mutations: K848A, K1003A, and R1060A. “eSpCas9(1.0)” refers to a modified SpCas9 protein with the following amino acid mutations: K810A, K1003A, and R1060A. The amino acid sequences for eSpCas9(1.1) and eSpCas9(1.0) are provided in Table 8 (i.e., SEQ ID NO: 245 and SEQ ID NO: 246, respectively). Additional details relating to eSpCas9(1.1) and eSpCas9(1.0) are provided in, e.g., Slaymaker et al., Science 351(6268): 84-88 (January 2016).

As used herein, the term “SpCas9-HF1” refers to a modified SpCas9 protein with the following amino acid mutations: N497A, R661A, Q695A, and Q926A. The term “SpCas9-HF4” refers to a modified SpCas9 protein which contains the amino acid mutations of SpCas9-HF1 and additionally has the Y450A amino acid mutation (i.e., has the following five mutations: N497A, Y450A, R661A, Q695A, and Q926A). The amino acid sequences for SpCas9-HF1 and Sp-Cas9-HF2 are provided in Table 8 (i.e., SEQ ID NOs: 247 and 248, respectively). See also Kleinstiver et al., Nature 529: 490-495 (2016).

The term “guide RNA” refers to an RNA molecule (or a group of RNA molecules collectively) that can bind to a Cas protein and aid in targeting the Cas protein to a specific location within a target polynucleotide (e.g., target sequence). Guide RNA can comprise a crRNA segment and a tracrRNA segment. As used herein, the term “crRNA” or “crRNA segment” refers to an RNA molecule or portion thereof that includes a polynucleotide-targeting guide sequence, a stem sequence, and, optionally, a 5-overhang sequence. As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The term “guide RNA” encompasses a single guide RNA (sgRNA), where the crRNA segment and the tracrRNA segment are located in the same RNA molecule. The term “guide RNA” also encompasses, collectively, a group of two or more RNA molecules, where the crRNA segment and the tracrRNA segment are located in separate RNA molecules.

As is apparent from the present disclosure, in combination with a Cas9 nuclease (such as those described herein), the guide RNAs facilitate the target specificity of the CRISPR/Cas9 system. Some aspects such as promoter choice can provide additional mechanisms of achieving target specificity—e.g., selecting a promoter for the guide RNA encoding polynucleotide that facilitates expression in a particular organ or tissue. Accordingly, the selection of suitable gRNAs for the particular disease, disorder, or condition is also contemplated and further described herein. As demonstrated herein, in some aspects, a gRNA useful for the present disclosure can be chemically synthesized (i.e., “synthetic gRNA”) to comprise a specific guide sequence. For example, in some aspects, a synthetic gRNA can comprise one or more base modifications (e.g., nucleotide substitution), such that the gRNA differs in sequence compared to a corresponding wild-type gRNA. Methods of constructing such synthetic gRNAs are provided elsewhere in the present disclosure (see, e.g., Example 1) and also described in, e.g., Doench, J., et al., Nature biotechnology 32(12): 1262-7 (2014); Mohr, S. et al., FEBS Journal 283: 3232-38 (2016); Graham, D., et al., Genome Biol. 16: 260 (2015); and Kelley, M. et al., J Biotechnology 233: 74-83 (2016); each of which is incorporated herein by reference in its entirety. In some aspects, the gRNAs described herein can comprise one or more modifications that further enhance the specificity of the Cas9 proteins described herein (e.g., shorter length of the target sequence (e.g., 18 nucleotides in length as opposed to 20 nucleotides long) or adding a guanine at the 5′-end of the gRNA). Additional examples of such modifications are known in the art.

As used herein, the term “target polynucleotide” or “target gene” (including variants thereof) refers to a polynucleotide containing a target nucleic acid sequence. A target polynucleotide can be single-stranded or double-stranded, and, in some aspects, is double-stranded DNA. In some aspects, the target polynucleotide is single-stranded RNA. A “target nucleic acid sequence” or “target sequence,” as used herein, refers to a sequence to which a gRNA is designed to bind to (e.g., complementary to the guide sequence of the gRNA), where the hybridization (or binding) between a target sequence and the guide sequence promotes the formation of a CRISPR complex, and the eventual cleaving of the sequence. Unless indicated otherwise, a target sequence can comprise any polynucleotide, such as DNA or RNA polynucleotides.

The term “hybridization” or “hybridizing” refers to a process where completely or partially complementary polynucleotide strands come together under suitable hybridization conditions to form a double-stranded structure or region in which the two constituent strands are joined by hydrogen bonds. As used herein, the term “partial hybridization” includes where the double-stranded structure or region contains one or more bulges or mismatches. Although hydrogen bonds typically form between adenine and thymine or adenine and uracil (A and T or A and U) or cytosine and guanine (C and G), other noncanonical base pairs can form (See e.g., Adams et al., “The Biochemistry of the Nucleic Acids,” 11th ed., 1992).

The term “CRISPR” refers to Clustered Regularly Interspaced Short Palindromic Repeats (CRISPR). In general, the CRISPR-Cas, CRISPR-Cas9 or CRISPR system is as used in the foregoing documents, such as US 2017/0152528, which is incorporated herein by reference in its entirety, and refers collectively to transcripts and other elements involved in the expression of or directing the activity of CRISPR-associated (“Cas”) genes, including sequences encoding a Cas gene, in particular a Cas9 gene in the case of CRISPR-Cas9, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system).

The term “protospacer adjacent motif” or “PAM” refers to a nucleotide sequence present in a target double-stranded polynucleotide (located adjacent to protospacers) that can be recognized by a Cas9 protein. Upon recognizing a PAM, the Cas9 protein opens up the double-stranded polynucleotide and determines whether the sequence adjacent to the PAM is complementary to the guide sequence of the gRNA. If the adjacent sequence is complementary, the Cas9 protein cleaves the target polynucleotide. Otherwise, the Cas9 protein continues along the target DNA strand looking for additional PAMs. PAM sequences and positions along a target DNA strand can vary according to the CRISPR-Cas system type. For example, in the S. pyogenes Type II system, the PAM has a NGG consensus sequence that contains two G:C base pairs and occurs one base pair downstream of the protospacer-derived sequence within the target DNA. The PAM sequence is present on the non-complementary strand of the target DNA (protospacer), and the reverse complement of the PAM is located 5′ of the target DNA sequence. The PAM sequence can be specific to the system, e.g., the system from which the site-directed modifying protein is derived.

As used herein, the term “specificity” (or also referred to as “fidelity”) refers to the ability of a Cas9 protein to specifically recognize and cleave a desired target sequence, whereas little or no cleaving of polynucleotides that are different in sequence and/or location from the desired target sequence. Thus, specificity refers to minimizing off-target effects and/or increasing on-target effects of a Cas9 protein. The activity (e.g., on-target and/or off-target activity) of a Cas9 protein, such as those described herein, can be assessed using the methods provided herein (see, e.g., Example 1) and/or any suitable methods known in the art. Non-limiting examples of such methods include: in vitro cleavage assay (see, e.g., worldwideweb.neb.com/protocols/2014/05/01/in-vitro-digestion-of-dna-with-cas9-nuclease-s-pyogenes-m0386, which is incorporated herein by reference in its entirety); Digenome-seq (see, e.g., Kim et al., Nature Methods 12:237-243 (2015), which is incorporated herein by reference in its entirety); GUIDE-seq (see, e.g., Tsai et al., Nat Biotechnol 33:187-197 (2015), which is incorporated herein by reference in its entirety); CIRCLE-seq (see, e.g., Tsai et al., Nature Methods 14:607-614 (2017), which is incorporated herein by reference in its entirety); or ChIP-seq (see, e.g., O'Geen et al., Nucleic Acids Res 43:3389-3404 (2015), which is incorporated herein by reference in its entirety). In some aspects, the rate of off-target effects can be assessed by determining the % of indels at an off-target site.

As used herein, the term “cleavage” refers to the breakage of the covalent backbone of a DNA molecule, e.g., caused by a Cas9 nuclease. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or cohesive ends.

As used herein, the term “target site” or “target sequence” refers to a region of a polynucleotide sequence to which a binding molecule can bind, provided sufficient conditions for binding exist (e.g., sufficient complementarity). In some aspects, a target sequence is a nucleic acid sequence to which a nuclease described herein (e.g., modified Cas9 protein) binds and/or that is cleaved by such nuclease. In some aspects, a target sequence is a nucleic acid sequence to which a guide RNA described herein binds. A target site can be single-stranded or double-stranded.

As will be apparent to those skilled in the art, the target sequence can vary depending on the nuclease being utilized. For example, in the context of RNA-guided nucleases (e.g., Cas9 protein described herein), a target sequence typically comprises a nucleotide sequence that is complementary to the guide sequence of a guide RNA of the RNA-programmable nuclease, and a protospacer adjacent motif (PAM) at the 3′ end or 5′ end adjacent to the guide RNA-complementary sequence. More specifically, for the RNA-guided nuclease Cas9, in some aspects, the target sequence can be about 16-24 base pairs in length plus a 3-6 base pair PAM (e.g., NNN, wherein N represents any nucleotide). As demonstrated herein, in some aspects, a target sequence of a Cas9 protein described herein is 20 base pairs in length (excluding the PAM).

In some aspects, the target sequence of a Cas9 protein described herein can comprise the structure [Nz]-[PAM], where each N is, independently, any nucleotide, and z is an integer between 1 and 50. In some aspects, z is at least about two, at least about three, at least about four, at least about five, at least about six, at least about seven, at least about eight, at least about nine, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, or at least about 50. In some aspects, Z is 20.

The term “off-target” refers to the binding and cleaving of an unintended or unexpected region of a polynucleotide (i.e., not at a target sequence) by a Cas9 nuclease. In some aspects, a region of a polynucleotide is an off-target region when it differs from the target region/sequence by: at least about one, at least about two, at least about three, at least about four, at least about five, at least about six, at least about seven, at least about eight, at least about nine, at least about 10, at least about 11, at least about 12, at least about 13, at least about 14, at least about 15, at least about 16, at least about 17, at least about 18, at least about 19, or at least about 20 or more nucleotides. As further described elsewhere, because of the enhanced specificity, in some aspects, the Cas9 proteins provided herein are associated with reduced off-target event.

As used herein, the term “on-target” refers to the binding and cleaving of an intended or expected region of a polynucleotide (e.g., target sequence) by a Cas9 nuclease.

The term “biomarker” refers to a protein or a nucleic acid (e.g., comprising a mutation) that causes and/or is associated with the presence of a particular disease or disorder.

As used herein, the terms “disease,” “disorder,” and “condition” (including any variants thereof) are used interchangeably and refer to an abnormal condition that negatively affects the structure or function of all or part of a subject, and that is not necessarily due to any immediate external injury. Generally, the diseases and disorders described herein are associated with specific signs and symptoms. Moreover, the diseases and disorders that can be diagnosed and/or treated using the present disclosure are not particularly limited. In some aspects, the disease or disorder is associated with an abnormal expression/activity of a gene (e.g., variant DNA pattern that is not present in the corresponding gene of a healthy subject), such that the disease or disorder can be diagnosed using the methods provided herein. In some aspects, the disease or disorder is associated with an abnormal expression/activity of a gene, such that the disease or disorder can be treated using the methods provided herein (e.g., by using a Cas9 protein described herein to delete or repair the mutated gene).

In some aspects, the disease or disorder that can be diagnosed and/or treated using the present disclosure comprises a cancer. Non-limiting examples of cancers include: a mesothelioma, cervical cancer, pancreatic cancer, ovarian cancer, squamous cell cancer (e.g. epithelial squamous cell cancer), lung cancer (e.g., small-cell lung cancer (SCLC), non-small cell lung cancer, adenocarcinoma of the lung and squamous carcinoma of the lung), skin cancer (e.g., basal cell carcinoma (BCC), cutaneous squamous cell carcinoma (cSCC), melanoma, Merkel cell carcinoma (MCC)), cancer of the peritoneum, hepatocellular cancer, gastric or stomach cancer (e.g., gastrointestinal cancer), esophageal cancer (e.g., gastroesophageal junction cancer), brain cancer (e.g., glioblastoma), liver cancer (e.g., hepatocellular carcinoma), bladder cancer, hepatoma, breast cancer (e.g., triple negative breast cancer (TNBC)), colon cancer, rectal cancer, colorectal cancer, endometrial cancer or uterine carcinoma, salivary gland carcinoma, kidney or renal cancer (e.g., renal cell carcinoma), prostate cancer, vulvar cancer, thyroid cancer, hepatic carcinoma, anal carcinoma, penile carcinoma, head and neck cancer (e.g., head and neck squamous cell carcinoma), biliary duct cancer, gall bladder cancer, muscular skeletal cancer, or any combination thereof. In some aspects, the cancer comprises a breast cancer, pancreatic cancer, colorectal cancer, or combinations thereof.

In some aspects, the disease or disorder that can be diagnosed and/or treated using the present disclosure comprises a neurodegenerative or neurologic disorder. Non-limiting examples of neurodegenerative or neurologic disorders include: an Alzheimer's disease, Parkinson's disease, amyotrophic lateral sclerosis, Friedreich's Ataxia, Huntington's disease, Lew body disease, spinal muscular atrophy, stroke or a combination thereof.

As used herein, the term “associated with” refers to a close relationship between two or more entities or properties. For instance, when used to describe a disease or condition that can be diagnosed with the present disclosure, the term “associated with” refers to an increased likelihood that a subject suffers from (i.e., afflicted with) the disease or condition when the subject exhibits an abnormal level of the biomarker. In some aspects, the abnormal expression causes the disease or condition. In some aspects, the abnormal expression does not necessarily cause but is correlated with the disease or condition. Non-limiting examples of suitable methods that can be used to determine whether a subject exhibits an abnormal expression of a biomarker associated with a disease or condition are provided elsewhere in the present disclosure.

The term “afflicted with” can be used interchangeably with the term “suffering from” and refers to the state of having a disease or condition. In some aspects, a subject afflicted with a disease or condition (e.g., cancer and/or neurodegenerative disease) exhibits one or more symptoms associated with the disease or condition. However, as will be apparent to those skilled in the art, a subject does not need to exhibit one or more symptoms to be afflicted with a disease or disorder disclosed herein (e.g., can have a genetic predisposition to the disease or disorder).

As used herein, the term “abnormal level” refers to a level (expression and/or activity) that differs (e.g., increased or decreased) from a reference subject, e.g., who does not suffer from a disease or condition described herein (e.g., cancer and/or neurodegenerative disease). In some aspects, an abnormal level (e.g., of a biomarker) refers to a level that is increased by at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 2-fold, at least about 3-fold, at least about 4-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, at least about 750-fold, or at least about 1,000-fold or more compared to the corresponding level in a reference subject (e.g., subject who does not suffer from a disease or condition described herein). In some aspects, an abnormal level (e.g., of a biomarker) refers to a level that is decreased by at least about 5%, at least about 10%, at least about 20%, at least about 30%, at least about 40%, at least about 50%, at least about 60%, at least about 70%, at least about 80%, at least about 90%, or about 100% compared to the corresponding level in a reference subject (e.g., subject who does not suffer from a disease or condition described herein).

As used herein, the term “diagnosing” (and derivatives thereof) refers to methods that can be used to determine or predict whether a subject is afflicted with, suffering from, or at a risk (e.g., genetically predisposed) for a given disease or condition, thereby identifying a subject who is suitable for a treatment. In some aspects, the treatment can be therapeutic (e.g., administered to a subject exhibiting one or more symptoms associated with the disease or disorder). In some aspects, the treatment can be prophylactic (e.g., administered to an at-risk subject to prevent and/or reduce the onset of the disease or disorder). As described herein, in some aspects, a skilled artisan can make a diagnosis on the basis of a biomarker, where the presence, absence, amount, or change in the amount of the biomarker is indicative of the presence, severity, or absence of the condition. The term “diagnosis” does not refer to the ability to determine the presence or absence of a particular disease or disorder with 100% accuracy, or even that a given course or outcome is more likely to occur than not. Instead, the skilled artisan will understand that the term “diagnosis” refers to an increased probability that a certain disease or disorder is present in the subject.

As used herein, the term “administering” (and grammatical variants thereof) refers to the physical introduction of a therapeutic agent (e.g., Cas9 protein described herein) or a composition comprising the therapeutic agent to a subject, using any of the various methods and delivery systems known to those skilled in the art. The different routes of administration include, but are not limited to, intravenous, intraperitoneal, intramuscular, subcutaneous, spinal, or other parenteral routes of administration, for example by injection or infusion.

“Parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent (e.g., Cas9 protein described herein) can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods. Administration also includes self-administration and the administration by another.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The terms “nucleic acids,” “nucleic acid molecules, “nucleotides,” “nucleotide(s) sequence,” and “polynucleotide” can be used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA).

The term “identity” or “sequence identity” refers to the overall monomer conservation between polymeric molecules, e.g., between polypeptides or between polynucleotides. The term “identical” without any additional qualifiers, e.g., polypeptide A is identical to polypeptide B, implies the polypeptide sequences are 100% identical (100% sequence identity). Describing two sequences as, e.g., “70% identical,” is equivalent to describing them as having, e.g., “70% sequence identity.”

Calculation of the percent identity of two polypeptides or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In some aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at worldwideweb.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. Length value will always be an integer.

In some aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at worldwidewebtcoffee.org, and alternatively available, e.g., from the EBI. It will also be appreciated that the final alignment used to calculate percent sequence identity can be curated either automatically or manually.

The term “variant” or “mutant” refers to a polypeptide comprising an amino acid sequence that is different from the reference polypeptide (e.g., corresponding Cas9 protein which has not been modified, e.g., wild-type Cas9 protein) by one or more amino acids, e.g., one or more amino acid substitutions, deletions, or additions. For example a modified or variant Cas9 polypeptide differs from wild-type Cas9 (e.g., SEQ ID NO: 1) by one or more amino acid substitutions, deletions, and/or additions, i.e., mutations. Unless indicated otherwise, such amino acid mutations are also referred to herein as “amino acid modifications.”

As used herein, the terms “isolated,” “purified,” “extracted,” and grammatical variants thereof, are used interchangeably and refer to the state of a preparation of desired composition of the present disclosure, e.g., Cas9 protein modified to exhibit enhanced specificity, that has undergone one or more processes of purification. In some aspects, isolating or purifying as used herein is the process of removing, or partially removing (e.g., a fraction), a composition of the present disclosure from a sample containing contaminants.

In some aspects, an isolated composition has no detectable undesired activity or, alternatively, the level or amount of the undesired activity is at or below an acceptable level or amount. An isolated composition can have an amount and/or concentration of desired composition of the present disclosure, at or above an acceptable amount and/or concentration and/or activity. In some aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. Cancers that can be treated with the present disclosure include those associated with a solid tumor.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

“Treatment” or “therapy” of a subject refers to any type of intervention or process performed on, or the administration of an active agent to, a subject with the objective of reversing, alleviating, ameliorating, inhibiting, slowing down, or preventing the onset, progression, development, severity, or recurrence of a symptom, complication, condition, or biochemical indicia associated with a disease.

As used herein, the terms “ug” and “uM” are used interchangeably with “μg” and “μM,” respectively. Various aspects described herein are described in further detail in the following subsections.

### II. Cas9 Protein Variants

Present disclosure provides Cas9 proteins with one or more improved properties compared to a reference Cas9 protein (e.g., wild-type Cas9 protein). For example, as demonstrated herein, the Cas9 proteins described herein comprise one or more amino acid modifications, such that the Cas9 proteins exhibit enhanced (or increased) specificity compared to a reference Cas9 protein. Accordingly, in some aspects, provided herein is a Cas9 protein comprising a cavity domain which comprises a plurality of amino acids, wherein at least one of the plurality of amino acids have been modified (“amino acid modification”) compared to a reference Cas9 protein (e.g., corresponding wild-type Cas9 protein), wherein the amino acid modification increases the specificity of the Cas9 protein. As used herein, the term “cavity domain” refers to the portion of a Cas9 protein that plays a role in the interaction of the Cas9 protein with a nucleic acid sequence.

A Cas9 protein, such as that exists in nature, comprises two lobes: a recognition (REC) lobe and a nuclease (NUC) lobe; each of which comprise particular structural and/or functional domains. The “REC lobe” comprises an arginine-rich bridge helix (BH) domain, and at least one REC domain (e.g., a REC1 domain and, optionally, a REC2 domain and a REC3 domain), which are involved in the Cas9 protein's recognition of the guide RNA scaffold and guide RNA/DNA heteroduplex. For example, while not wishing to be bound by any one theory, in some aspects, the BH domain plays a role in gRNA:DNA recognition, while the REC domain interacts with the repeat:anti-repeat duplex of the gRNA and to mediate the formation of the Cas9/gRNA complex. The “NUC lobe” comprises a RuvC domain, a HNH domain, and a PAM-interacting (PI) domain. The RuvC domain is primarily responsible for cleaving the non-complementary (i.e., bottom or non-target) strand of the target nucleic acid. The HNH domain, meanwhile, is responsible for cleaving the complementary (i.e., top or target strand) strand of the target nucleic acid. The PI domain contributes to PAM specificity. The term “cavity domain,” as used herein comprises both the REC and NUC lobes. As further described elsewhere in the present disclosure, Applicant has identified that modifying one or more amino acids within the cavity domain of a Cas9 protein can enhance the specificity of the Cas9 protein.

In some aspects, compared to a reference Cas9 protein (e.g., corresponding wild-type Cas9 protein), the specificity of a Cas9 protein is increased by at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold or more.

As is apparent from the present disclosure, the enhanced specificity allows the Cas9 proteins of the present disclosure to more accurately recognize base mismatches within a nucleic acid sequence (e.g., sequence of a target gene to be modified). Not to be bound by any one theory, as a result, in some aspects, the Cas9 proteins described herein are associated with reduced off-target effects (e.g., off-target binding, editing, and/or cleavage activity), as they are capable of not cleaving sequences comprising such base mismatches. Similarly, in some aspects, such Cas9 proteins can have increased on-target effects (e.g., on-target binding, editing, and/or cleavage activity). As further described elsewhere in the present disclosure, such base mismatches can be present within the target sequence to which the gRNA binds to. In some aspects, the base mismatches can occur within a PAM. In some aspects, the base mismatches can occur both within the target sequence and within the PAM.

In some aspects, a Cas9 protein of the present disclosure can accurately distinguish a nucleic acid sequence comprising multiple base mismatches (e.g., within the target sequence and/or the PAM). For example, in some aspects, a Cas9 protein described herein can recognize a nucleic acid sequence comprising at least two, at least three, at least four, at least five, at least six, at least seven, at least eight, at least nine, or at least 10 or more base mismatches, and thereby, not cleave such a sequence. As described herein, where multiple base mismatches are present, in some aspects, the multiple base mismatches can all be present within the target sequence. In some aspects, the multiple base mismatches can all be present within the PAM. In some aspects, some of the multiple base mismatches can be within the target sequence, while some of the multiple base mismatches can be within the PAM. In some aspects, as demonstrated herein, a Cas9 protein described herein can recognize (and thereby, not cleave) a nucleic acid sequence having a single base mismatch. In some aspects, the single base mismatch can be within the target sequence. In some aspects, the single base mismatch can be within the PAM. Accordingly, in some aspects, a Cas9 protein of the present disclosure is capable of cleaving only nucleic acid sequences that are fully complementary (i.e., 100% complementary) to the guide sequence of the gRNA.

As described herein, in some aspects, a Cas9 protein of the present disclosure comprises at least about one, at least about two, at least about three, at least about four, at least about five, at least about six, at least about seven, at least about eight, at least about nine, at least about 10, at least about 11, at least about 12, at least about 13, at least about 14, at least about 15, at least about 16, at least about 17, at least about 18, at least about 19, or at least about 20 or more amino acid modifications (e.g., substitutions) within the cavity domain of the Cas9 protein.

In some aspects, a Cas9 protein described herein comprises about one amino acid modification within the cavity domain. In some aspects, a Cas9 protein described herein comprises about two amino acid modifications within the cavity domain. In some aspects, a Cas9 protein described herein comprises about three amino acid modifications within the cavity domain. In some aspects, a Cas9 protein described herein comprises about four amino acid modifications within the cavity domain. In some aspects, a Cas9 protein described herein comprises about five amino acid modifications within the cavity domain. In some aspects, a Cas9 protein described herein comprises about six amino acid modifications within the cavity domain. In some aspects, a Cas9 protein described herein comprises about seven amino acid modifications within the cavity domain. In some aspects, a Cas9 protein described herein comprises about eight amino acid modifications within the cavity domain. In some aspects, a Cas9 protein described herein comprises about nine amino acid modifications within the cavity domain. In some aspects, a Cas9 protein described herein comprises about 10 amino acid modifications within the cavity domain. In some aspects, a Cas9 protein described herein comprises about 11 amino acid modifications within the cavity domain. In some aspects, a Cas9 protein described herein comprises about 12 amino acid modifications within the cavity domain. In some aspects, a Cas9 protein described herein comprises about 13 amino acid modifications within the cavity domain. In some aspects, a Cas9 protein described herein comprises about 14 amino acid modifications within the cavity domain. In some aspects, a Cas9 protein described herein comprises about 15 amino acid modifications within the cavity domain. In some aspects, a Cas9 protein described herein comprises about 16 amino acid modifications within the cavity domain. In some aspects, a Cas9 protein described herein comprises about 17 amino acid modifications within the cavity domain. In some aspects, a Cas9 protein described herein comprises about 18 amino acid modifications within the cavity domain. In some aspects, a Cas9 protein described herein comprises about 19 amino acid modifications within the cavity domain. In some aspects, a Cas9 protein described herein comprises about 20 amino acid modifications within the cavity domain.

As demonstrated herein, Applicant has identified that modifications (e.g., substitution) to certain amino acid residues within the cavity domain of a Cas9 protein can increase the specificity of the Cas9 protein. For example, in some aspects, an amino acid residue that is modified is capable of interacting with a backbone phosphate of a target DNA strand (i.e., strand of a DNA molecule to which the Cas9-gRNA complex binds and cleaves) (e.g., within the REC lobe). In some aspects, an amino acid residue that is modified is capable of interacting with a backbone phosphate of a non-target DNA strand (e.g., within the NUC lobe). Where multiple amino acid residues within the cavity domain of a Cas9 protein are modified, in some aspects, all the modified amino acid residues are those that are capable of interacting with a backbone phosphate of a target DNA strand. In some aspects, all the modified amino acid residues are those that are capable of interacting with a backbone phosphate of a non-target DNA strand. In some aspects, the multiple amino acid residues that are modified include both those that interact with a backbone phosphate of a target DNA strand and those that interact with a backbone phosphate of a non-target DNA strand. Not to be bound by any one theory, in some aspects, modifying such amino acid residues that are involved in the interaction between a Cas9 protein and a target DNA can help improve the specificity of the Cas9 protein. In some aspects, an amino acid residue that can be modified comprises a positively-charged amino acid, such as a histidine (H), lysine (K), arginine (R), or a combination thereof. Additional details relating to such modifications are provided elsewhere in the present disclosure.

Methods of identifying amino acid residues of a Cas9 protein that are involved in DNA interaction are known in the art. Non-limiting examples of such methods include crystallography, nuclear magnetic resonance (NMR), and sequence aligner. Accordingly, while the present disclosure largely focuses on Cas9 protein from Francisella novicida (FnCas9), it will be apparent to those skilled in the art that the disclosures provided herein can equally apply to Cas9 proteins from other sources. For example, in some aspects, a Cas9 protein that can be modified using the present disclosure to enhance specificity comprises Cas9 protein from Streptococcus pyogenes (SpCas9). Non-limiting examples of other suitable Cas9 proteins are known in the art. See, e.g., Gasiunas et al., Nat Commun 11(1): 5512 (November 2020), which is incorporated herein by reference in its entirety. In some aspects, a Cas9 protein described herein comprises a split Cas9 molecule or an inducible Cas9 molecule, as described in, e.g., WO 2015/089427 and WO 2014/018423, each of which is incorporated herein by reference in its entirety.

In some aspects, a Cas9 protein described herein comprises the amino acid sequence set forth in SEQ ID NO: 1, wherein one or more of the following amino acid residues are modified (e.g., substitution) relative to SEQ ID NO: 1: K405, R455, K546, K561, K562, K564, K566, K578, K579, R618, R622, K664, R721, R785, K786, K788, K789, R807, K808, R849, R856, K914, K917, R919, R920, K921, K922, R926, K934, K936, R939, K941, K945, R1047, R1131, R1137, K1142, K1152, K1155, R1178, K1189, K1198, K1206, K1213, K1223, R1226, K1227, K1228, R1241, or a combination thereof.

In some aspects, a Cas9 protein described herein comprises the amino acid sequence set forth in SEQ ID NO: 1, wherein one or more of the following amino acid residues are modified (e.g., substitution) relative to SEQ ID NO: 1: K405, R455, K566, K578, K664, R721, R785, K786, K789, K914, K917, R919, K921, K922, R926, K934, K936, R939, K941, K945, R1137, K1142, K1152, K1189, K1198, K1206, K1223, R1226, K1227, K1228, R1241, or a combination thereof.

In some aspects, a Cas9 protein described herein comprises the amino acid sequence set forth in SEQ ID NO: 1, wherein one or more of the following amino acid residues are modified (e.g., substitution) relative to SEQ ID NO: 1: R785, K789, R455, R721, R919, R1241, R939, K1189, K941, R1226, K1228, or a combination thereof.

In some aspects, a Cas9 protein described herein comprises the amino acid sequence set forth in SEQ ID NO: 1, wherein the amino acid residue K405 is modified relative to SEQ ID NO: 1. In some aspects, a Cas9 protein described herein comprises the amino acid sequence set forth in SEQ ID NO: 1, wherein the amino acid residue R455 is modified relative to SEQ ID NO: 1. In some aspects, a Cas9 protein described herein comprises the amino acid sequence set forth in SEQ ID NO: 1, wherein the amino acid residue K546 is modified relative to SEQ ID NO: 1. In some aspects, a Cas9 protein described herein comprises the amino acid sequence set forth in SEQ ID NO: 1, wherein the amino acid residue K561 is modified relative to SEQ ID NO: 1. In some aspects, a Cas9 protein described herein comprises the amino acid sequence set forth in SEQ ID NO: 1, wherein the amino acid residue K562 is modified relative to SEQ ID NO: 1. In some aspects, a Cas9 protein described herein comprises the amino acid sequence set forth in SEQ ID NO: 1, wherein the amino acid residue K564 is modified relative to SEQ ID NO: 1. In some aspects, a Cas9 protein described herein comprises the amino acid sequence set forth in SEQ ID NO: 1, wherein the amino acid residue K566 is modified relative to SEQ ID NO: 1. In some aspects, a Cas9 protein described herein comprises the amino acid sequence set forth in SEQ ID NO: 1, wherein the amino acid residue K578 is modified relative to SEQ ID NO: 1. In some aspects, a Cas9 protein described herein comprises the amino acid sequence set forth in SEQ ID NO: 1, wherein the amino acid residue K579 is modified relative to SEQ ID NO: 1. In some aspects, a Cas9 protein described herein comprises the amino acid sequence set forth in SEQ ID NO: 1, wherein the amino acid residue R618 is modified relative to SEQ ID NO: 1. In some aspects, a Cas9 protein described herein comprises the amino acid sequence set forth in SEQ ID NO: 1, wherein the amino acid residue R622 is modified relative to SEQ ID NO: 1. In some aspects, a Cas9 protein described herein comprises the amino acid sequence set forth in SEQ ID NO: 1, wherein the amino acid residue K664 is modified relative to SEQ ID NO: 1. In some aspects, a Cas9 protein described herein comprises the amino acid sequence set forth in SEQ ID NO: 1, wherein the amino acid residue R721 is modified relative to SEQ ID NO: 1. In some aspects, a Cas9 protein described herein comprises the amino acid sequence set forth in SEQ ID NO: 1, wherein the amino acid residue R785 is modified relative to SEQ ID NO: 1. In some aspects, a Cas9 protein described herein comprises the amino acid sequence set forth in SEQ ID NO: 1, wherein the amino acid residue K786 is modified relative to SEQ ID NO: 1. In some aspects, a Cas9 protein described herein comprises the amino acid sequence set forth in SEQ ID NO: 1, wherein the amino acid residue K788 is modified relative to SEQ ID NO: 1. In some aspects, a Cas9 protein described herein comprises the amino acid sequence set forth in SEQ ID NO: 1, wherein the amino acid residue K789 is modified relative to SEQ ID NO: 1. In some aspects, a Cas9 protein described herein comprises the amino acid sequence set forth in SEQ ID NO: 1, wherein the amino acid residue R807 is modified relative to SEQ ID NO: 1. In some aspects, a Cas9 protein described herein comprises the amino acid sequence set forth in SEQ ID NO: 1, wherein the amino acid residue K808 is modified relative to SEQ ID NO: 1. In some aspects, a Cas9 protein described herein comprises the amino acid sequence set forth in SEQ ID NO: 1, wherein the amino acid residue R849 is modified relative to SEQ ID NO: 1. In some aspects, a Cas9 protein described herein comprises the amino acid sequence set forth in SEQ ID NO: 1, wherein the amino acid residue R856 is modified relative to SEQ ID NO: 1. In some aspects, a Cas9 protein described herein comprises the amino acid sequence set forth in SEQ ID NO: 1, wherein the amino acid residue K914 is modified relative to SEQ ID NO: 1. In some aspects, a Cas9 protein described herein comprises the amino acid sequence set forth in SEQ ID NO: 1, wherein the amino acid residue K917 is modified relative to SEQ ID NO: 1. In some aspects, a Cas9 protein described herein comprises the amino acid sequence set forth in SEQ ID NO: 1, wherein the amino acid residue R919 is modified relative to SEQ ID NO: 1. In some aspects, a Cas9 protein described herein comprises the amino acid sequence set forth in SEQ ID NO: 1, wherein the amino acid residue R920 is modified relative to SEQ ID NO: 1. In some aspects, a Cas9 protein described herein comprises the amino acid sequence set forth in SEQ ID NO: 1, wherein the amino acid residue K921 is modified relative to SEQ ID NO: 1. In some aspects, a Cas9 protein described herein comprises the amino acid sequence set forth in SEQ ID NO: 1, wherein the amino acid residue K922 is modified relative to SEQ ID NO: 1. In some aspects, a Cas9 protein described herein comprises the amino acid sequence set forth in SEQ ID NO: 1, wherein the amino acid residue R926 is modified relative to SEQ ID NO: 1. In some aspects, a Cas9 protein described herein comprises the amino acid sequence set forth in SEQ ID NO: 1, wherein the amino acid residue K934 is modified relative to SEQ ID NO: 1. In some aspects, a Cas9 protein described herein comprises the amino acid sequence set forth in SEQ ID NO: 1, wherein the amino acid residue K936 is modified relative to SEQ ID NO: 1. In some aspects, a Cas9 protein described herein comprises the amino acid sequence set forth in SEQ ID NO: 1, wherein the amino acid residue R939 is modified relative to SEQ ID NO: 1. In some aspects, a Cas9 protein described herein comprises the amino acid sequence set forth in SEQ ID NO: 1, wherein the amino acid residue K941 is modified relative to SEQ ID NO: 1. In some aspects, a Cas9 protein described herein comprises the amino acid sequence set forth in SEQ ID NO: 1, wherein the amino acid residue K945 is modified relative to SEQ ID NO: 1. In some aspects, a Cas9 protein described herein comprises the amino acid sequence set forth in SEQ ID NO: 1, wherein the amino acid residue R1047 is modified relative to SEQ ID NO: 1. In some aspects, a Cas9 protein described herein comprises the amino acid sequence set forth in SEQ ID NO: 1, wherein the amino acid residue R1131 is modified relative to SEQ ID NO: 1. In some aspects, a Cas9 protein described herein comprises the amino acid sequence set forth in SEQ ID NO: 1, wherein the amino acid residue R1137 is modified relative to SEQ ID NO: 1. In some aspects, a Cas9 protein described herein comprises the amino acid sequence set forth in SEQ ID NO: 1, wherein the amino acid residue K1142 is modified relative to SEQ ID NO: 1. In some aspects, a Cas9 protein described herein comprises the amino acid sequence set forth in SEQ ID NO: 1, wherein the amino acid residue K1152 is modified relative to SEQ ID NO: 1. In some aspects, a Cas9 protein described herein comprises the amino acid sequence set forth in SEQ ID NO: 1, wherein the amino acid residue K1155 is modified relative to SEQ ID NO: 1. In some aspects, a Cas9 protein described herein comprises the amino acid sequence set forth in SEQ ID NO: 1, wherein the amino acid residue R1178 is modified relative to SEQ ID NO: 1. In some aspects, a Cas9 protein described herein comprises the amino acid sequence set forth in SEQ ID NO: 1, wherein the amino acid residue K1189 is modified relative to SEQ ID NO: 1. In some aspects, a Cas9 protein described herein comprises the amino acid sequence set forth in SEQ ID NO: 1, wherein the amino acid residue K1198 is modified relative to SEQ ID NO: 1. In some aspects, a Cas9 protein described herein comprises the amino acid sequence set forth in SEQ ID NO: 1, wherein the amino acid residue K1206 is modified relative to SEQ ID NO: 1. In some aspects, a Cas9 protein described herein comprises the amino acid sequence set forth in SEQ ID NO: 1, wherein the amino acid residue K1213 is modified relative to SEQ ID NO: 1. In some aspects, a Cas9 protein described herein comprises the amino acid sequence set forth in SEQ ID NO: 1, wherein the amino acid residue K1223 is modified relative to SEQ ID NO: 1. In some aspects, a Cas9 protein described herein comprises the amino acid sequence set forth in SEQ ID NO: 1, wherein the amino acid residue R1226 is modified relative to SEQ ID NO: 1. In some aspects, a Cas9 protein described herein comprises the amino acid sequence set forth in SEQ ID NO: 1, wherein the amino acid residue K1227 is modified relative to SEQ ID NO: 1. In some aspects, a Cas9 protein described herein comprises the amino acid sequence set forth in SEQ ID NO: 1, wherein the amino acid residue K1228 is modified relative to SEQ ID NO: 1. In some aspects, a Cas9 protein described herein comprises the amino acid sequence set forth in SEQ ID NO: 1, wherein the amino acid residue R1241 is modified relative to SEQ ID NO: 1.

As described herein, in some aspects, a Cas9 protein of the present disclosure comprises multiple amino acid modifications (e.g., substitutions). In some aspects, a Cas9 protein described herein comprises two amino acid modifications. For example, in some aspects, a Cas9 protein described herein comprises the amino acid sequence set forth in SEQ ID NO: 1, wherein the amino acid residues K1189 and R1241 are modified relative to SEQ ID NO: 1. In some aspects, a Cas9 protein described herein comprises the amino acid sequence set forth in SEQ ID NO: 1, wherein the amino acid residues R721 and R1241 are modified relative to SEQ ID NO: 1. In some aspects, a Cas9 protein described herein comprises the amino acid sequence set forth in SEQ ID NO: 1, wherein the amino acid residues R785 and R1241 are modified relative to SEQ ID NO: 1. In some aspects, a Cas9 protein described herein comprises three amino acid modifications. For example, in some aspects, a Cas9 protein described herein comprises the amino acid sequence set forth in SEQ ID NO: 1, wherein the amino acid residues R785, K1189, and R1241 are modified relative to SEQ ID NO: 1. In some aspects, a Cas9 protein described herein comprises the amino acid sequence set forth in SEQ ID NO: 1, wherein the amino acid residues R721, K1189, and R1241 are modified relative to SEQ ID NO: 1. In some aspects, a Cas9 protein described herein comprises the amino acid sequence set forth in SEQ ID NO: 1, wherein the amino acid residues K1189, K1228, and R1241 are modified relative to SEQ ID NO: 1. In some aspects, a Cas9 protein described herein comprises the amino acid sequence set forth in SEQ ID NO: 1, wherein at least two different amino acid residues are modified relative to SEQ ID NO: 1, and wherein the at least two different amino acid residues are independently selected from: K405, R455, K546, K561, K562, K564, K566, K578, K579, R618, R622, K664, R721, R785, K786, K788, K789, R807, K808, R849, R856, K914, K917, R919, R920, K921, K922, R926, K934, K936, R939, K941, K945, R1047, R1131, R1137, K1142, K1152, K1155, R1178, K1198, K1206, K1213, K1223, R1226, K1227, K1228, R1241, or K1189.

As is apparent from the present disclosure, a Cas9 protein described herein can comprise any suitable amino modifications, as long as one or more of the amino acid modifications can enhance the specificity of the Cas9 protein. Non-limiting examples of such modifications include a substitution, deletion, insertion, or a combination thereof. As demonstrated herein, in some aspects, one or more of the amino acid residues described herein are replaced or substituted with a different amino acid. In some aspects, a suitable modification comprises a conservative substitution. “Conservative substitution” (also referred to as conservative replacement) as used herein means an amino acid replacement that changes a given amino acid to a different amino acid with similar biochemical properties (e.g., charge, hydrophobicity and size). Although there are many ways to classify amino acids, they are often sorted into six main groups on the basis of their structure and the general chemical characteristics of their R groups. In some aspects, a suitable modification comprises a radical substitution. As used herein, the term “radical substitution” refers to an amino acid replacement that exchanges an initial amino acid by a final amino acid with different physicochemical properties.

TABLE 2
Amino Acid Classes

| Class | Amino Acids |
|---|---|
| Aliphatic | Glycine, Alanine, Valine, Leucine, |
| | Isoleucine |
| Hydroxyl or | Serine, Cysteine, Selenocysteine, |
| sulfur/ | Threonine, Methionine |
| selenium- | |
| containing | |
| Cyclic | Proline |
| Aromatic | Phenylalanine, Tyrosine, Tryptophan |
| Basic | Histidine, Lysine, Arginine |
| (positively- | |
| charged) | |
| Acidic and | Aspartate, Glutamate, Asparagine, |
| their amides | Glutamine |

Where a Cas9 protein comprises multiple amino acid modifications, in some aspects, one or more of the multiple amino acid modifications comprise conservative substitutions. In some aspects, one or more of the multiple amino acid modifications comprise radical substitutions. In some aspects, the multiple amino acid modifications comprise a conservative substitution and a radical substitution.

In some aspects, one or more of the amino acid residues provided herein is substituted with an aliphatic amino acid. For example, in some aspects, one or more of the positively-charged amino acids present within the cavity domain of a Cas9 protein is substituted with an aliphatic amino acid. In some aspects, the aliphatic amino acid comprises an alanine.

Accordingly, in some aspects, a Cas9 protein described herein comprises the amino acid sequence set forth in SEQ ID NO: 1, wherein one or more of the following positively-charged amino acid residues of SEQ ID NO: 1 are substituted with an aliphatic amino acid: K405, R455, K566, K578, K664, R721, R785, K786, K789, K914, K917, R919, K921, K922, R926, K934, K936, R939, K941, K945, R1137, K1142, K1152, K1189, K1198, K1206, K1223, R1226, K1227, K1228, R1241, or a combination thereof.

In some aspects, a Cas9 protein described herein comprises a K405A substitution (e.g., SEQ ID NO: 251). In some aspects, a Cas9 protein described herein comprises a R455A substitution (e.g., SEQ ID NO: 252). In some aspects, a Cas9 protein described herein comprises a K566A substitution (e.g., SEQ ID NO: 253). In some aspects, a Cas9 protein described herein comprises a K578A substitution (e.g., SEQ ID NO: 254). In some aspects, a Cas9 protein described herein comprises a K664A substitution (e.g., SEQ ID NO: 255). In some aspects, a Cas9 protein described herein comprises a R721A substitution (e.g., SEQ ID NO: 256). In some aspects, a Cas9 protein described herein comprises a R785A substitution (e.g., SEQ ID NO: 257). In some aspects, a Cas9 protein described herein comprises a K786A substitution (e.g., SEQ ID NO: 258). In some aspects, a Cas9 protein described herein comprises a K789A substitution (e.g., SEQ ID NO: 259). In some aspects, a Cas9 protein described herein comprises a K914A substitution (e.g., SEQ ID NO: 260). In some aspects, a Cas9 protein described herein comprises a K917A substitution (e.g., SEQ ID NO: 261). In some aspects, a Cas9 protein described herein comprises a R919A substitution (e.g., SEQ ID NO: 262). In some aspects, a Cas9 protein described herein comprises a K921A substitution (e.g., SEQ ID NO: 263). In some aspects, a Cas9 protein described herein comprises a K922A substitution (e.g., SEQ ID NO: 264). In some aspects, a Cas9 protein described herein comprises a R926A substitution (e.g., SEQ ID NO: 265). In some aspects, a Cas9 protein described herein comprises a K934A substitution (e.g., SEQ ID NO: 266). In some aspects, a Cas9 protein described herein comprises a K936A substitution (e.g., SEQ ID NO: 267). In some aspects, a Cas9 protein described herein comprises a R939A substitution (e.g., SEQ ID NO: 268). In some aspects, a Cas9 protein described herein comprises a K941A substitution (e.g., SEQ ID NO: 269). In some aspects, a Cas9 protein described herein comprises a K945A substitution (e.g., SEQ ID NO: 270). In some aspects, a Cas9 protein described herein comprises a R1137A substitution (e.g., SEQ ID NO: 271). In some aspects, a Cas9 protein described herein comprises a K1142A substitution (e.g., SEQ ID NO: 272). In some aspects, a Cas9 protein described herein comprises a K1152A substitution (e.g., SEQ ID NO: 273). In some aspects, a Cas9 protein described herein comprises a K1189A substitution (e.g., SEQ ID NO: 2). In some aspects, a Cas9 protein described herein comprises a K1198A substitution (e.g., SEQ ID NO: 274). In some aspects, a Cas9 protein described herein comprises a K1206A substitution (e.g., SEQ ID NO: 275). In some aspects, a Cas9 protein described herein comprises a K1223A substitution (e.g., SEQ ID NO: 276). In some aspects, a Cas9 protein described herein comprises a R1226A substitution (e.g., SEQ ID NO: 277). In some aspects, a Cas9 protein described herein comprises a K1227A substitution (e.g., SEQ ID NO: 278). In some aspects, a Cas9 protein described herein comprises a K1228A substitution (e.g., SEQ ID NO: 279). In some aspects, a Cas9 protein described herein comprises a R1241A substitution (e.g., SEQ ID NO: 3).

In some aspects, a Cas9 protein described herein comprises multiple amino acid modifications, wherein the multiple amino acid modifications comprise K1189A and R1241A substitutions. Accordingly, in some aspects, a Cas9 protein described herein comprises the amino acid sequence set forth in SEQ ID NO: 4. In some aspects, a Cas9 protein described herein consists of the amino acid sequence set forth in SEQ ID NO: 4. In some aspects, a Cas9 protein described herein consists essentially of the amino acid sequence set forth in SEQ ID NO: 4

In some aspects, the multiple modifications comprise R721A and R1241A substitutions. Accordingly, in some aspects, a Cas9 protein described herein comprises the amino acid sequence set forth in SEQ ID NO: 5. In some aspects, a Cas9 protein described herein consists of the amino acid sequence set forth in SEQ ID NO: 5. In some aspects, a Cas9 protein described herein consists essentially of the amino acid sequence set forth in SEQ ID NO: 5.

In some aspects, the multiple modifications comprise R785A and R1241A substitutions. Accordingly, in some aspects, a Cas9 protein described herein comprises the amino acid sequence set forth in SEQ ID NO: 6. In some aspects, a Cas9 protein described herein consists of the amino acid sequence set forth in SEQ ID NO: 6. In some aspects, a Cas9 protein described herein consists essentially of the amino acid sequence set forth in SEQ ID NO: 6.

In some aspects, the multiple modifications comprise R785A, K1189A, and R1241A substitutions. Accordingly, a Cas9 protein described herein can comprise the amino acid sequence set forth in SEQ ID NO: 7. In some aspects, a Cas9 protein described herein consists of the amino acid sequence set forth in SEQ ID NO: 7. In some aspects, a Cas9 protein described herein consists essentially of the amino acid sequence set forth in SEQ ID NO: 7.

In some aspects, the multiple modifications comprise R721A, K1189A, and R1241A substitutions. Accordingly, a Cas9 protein described herein can comprise the amino acid sequence set forth in SEQ ID NO: 8. In some aspects, a Cas9 protein described herein consists of the amino acid sequence set forth in SEQ ID NO: 8. In some aspects, a Cas9 protein described herein consists essentially of the amino acid sequence set forth in SEQ ID NO: 8.

In some aspects, the multiple modifications comprise K1189A, K1228A, and R1241A substitutions. Accordingly, a Cas9 protein described herein can comprise the amino acid sequence set forth in SEQ ID NO: 9. In some aspects, a Cas9 protein described herein consists of the amino acid sequence set forth in SEQ ID NO: 9. In some aspects, a Cas9 protein described herein consists essentially of the amino acid sequence set forth in SEQ ID NO: 9.

As described herein, in some aspects, a Cas9 protein described herein can be a fusion protein. For example, in some aspects, the Cas9 protein can be conjugated (e.g., directly or via a linker) or fused to an agent (e.g., heterologous peptide). Any suitable agents known in the art can be conjugated or fused to a Cas9 protein described herein to produce the fusion protein. For instance, in some aspects, a Cas9 protein described herein is fused to a therapeutic agent, which can be useful in treating a disease or disorder, such as that described herein. In some aspects, a Cas9 protein described herein is conjugated to a guide RNA to form a Cas9:guide RNA complex. In some aspects, a Cas9 protein described herein is conjugated or fused to an agent that aids in improving the activity of the Cas9 protein. For example, in some aspects, the Cas9 protein is conjugated or fused to a nuclear localization signal and/or a cell penetrating amino acid sequence, such that the Cas9 protein can more effectively penetrate into a cell (or the nucleus of a cell). In some aspects, the Cas9 protein can be conjugated or fused to a tag, e.g., affinity/purification tag or a detectable tag, which can be useful, for instance, in producing the Cas9 protein or in determining whether a cell comprises the Cas9 protein. Non-limiting examples of such tags include: β-galactosidase, glutathione-S-transferase, green fluorescent proteins (GFP), epitope tags such as FLAG, myc tag, polyhistidine, nuclease (exo-, endo-) transcription factor, zinc-finger, TAL, deaminase, transposase, methyltransferase, single strand DNA binding protein (SSB), and intein.

### III. Polynucleotides, Vectors, and Cells

Some aspects of the present disclosure relates to polynucleotides (e.g., isolated polynucleotides) encoding any of the Cas9 protein variants described herein (or a functional fragment thereof). The polynucleotides can be present in whole cells, in a cell lysate, or in a partially purified or substantially pure form. A polynucleotide is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. A nucleic acid described herein can be, for example, DNA or RNA and can or cannot contain intronic sequences. In some aspects, the nucleic acid is a cDNA molecule. Nucleic acids described herein can be obtained using standard molecular biology techniques known in the art.

Exemplary polynucleotides encoding RNA-guided nucleases (e.g., wild-type Cas9 protein) have been described previously (see, e.g., Cong et al., Science 339(6121):819-23 (February 2013); Wang et al., PLoS One 8(12):e85650 (December 2013); each of which is incorporated herein by reference in its entirety). As is apparent from the present disclosure, polynucleotides useful for the present disclosure differ from such exemplary polynucleotides (e.g., in sequence), as the present polynucleotides encode Cas9 proteins comprising one or more of the amino acid modifications described herein. Accordingly, in some aspects, an isolated polynucleotide provided herein comprises a nucleic acid sequence that has less than about 99.999%, less than about 99.998%, less than about 99.997%, less than about 99.996%, less than about 99.995%, less than about 99.994%, less than about 99.993%, less than about 99.992%, less than about 99.991%, less than about 99.99%, less than about 99.8%, less than about 99.7%, less than about 99.6%, less than about 99.5%, less than about 99.4%, less than about 99.3%, less than about 99.2%, less than about 99.1%, less than about 99%, less than about 98%, less than about 97%, less than about 96%, or less than about 95% sequence identity to the nucleic acid set forth in SEQ ID NO: 2.

In some aspects, a polynucleotide described herein (encoding a Cas9 protein of the present disclosure) can comprise at least one chemically modified nucleobase, sugar, backbone, or any combination thereof. Thus, a polynucleotide encoding the Cas9 protein of the present disclosure can comprise one or more modifications.

In some aspects, the present disclosure provides a vector comprising an isolated polynucleotide encoding a Cas9 protein with enhanced specificity, such as those described herein.

Suitable vectors for the disclosure include expression vectors, viral vectors, and plasmid vectors. In some aspects, the vector is a viral vector.

Viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus (AAV). In some aspects, a vector is derived from a lentivirus. Examples of the lentiviral vectors are disclosed in WO9931251, W09712622, W09817815, W09817816, and WO9818934, each of which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

In some aspects, the present disclosure is directed to a cell comprising any of the Cas9 proteins, polynucleotides, or vectors described herein. As further described elsewhere in the present disclosure, in some aspects, a cell that has been modified (e.g., transduced) to comprise an isolated polynucleotide encoding a Cas9 protein described herein or a vector comprising the polynucleotide can be useful in producing the Cas9 proteins described herein. As also further described elsewhere in the present disclosure, in some aspects, a cell that has been modified to comprise any of the Cas9 proteins, polynucleotides, or vectors described herein can be useful in treating a disease or disorder (e.g., as part of a gene therapy).

### IV. Pharmaceutical Compositions

Provided herein are compositions comprising a Cas9 protein described herein (e.g., having enhanced specificity) (or an isolated polynucleotide, vector, or cell relating to such a Cas9 protein) having the desired degree of purity, and a pharmaceutically acceptable carrier or excipient, in a form suitable for administration to a subject. In some aspects, the composition further comprises a guide RNA, wherein the guide RNA is capable of interacting with the Cas9 protein and guiding the Cas9 protein to the target sequence.

Pharmaceutically acceptable excipients or carriers can be determined in part by the particular composition being administered, as well as by the particular method used to administer the composition. Accordingly, there is a wide variety of suitable formulations of pharmaceutical compositions (See, e.g., Remington, 23^{rd} Edition, The Science and Practice of Pharmacy, editor: A. Adejare, 2020, Academic Press.). The pharmaceutical compositions are generally formulated sterile and in full compliance with all Good Manufacturing Practice (GMP) regulations of any applicable Food and Drug Administration.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a pharmaceutical composition disclosed herein comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also be dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a IVIES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a pharmaceutical composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a pharmaceutical composition disclosed herein can also comprise a stabilizing agent. Non-limiting examples of stabilizing agents that can be used with the present disclosure include: sucrose, trehalose, raffinose, arginine, or combinations thereof.

In some aspects, a pharmaceutical composition disclosed herein comprises a surfactant. The surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a pharmaceutical composition disclosed herein further comprises an amino acid. The amino acid can be selected from arginine, glutamate, glycine, histidine, or combinations thereof. In some aspects, the composition further comprises a sugar alcohol. Examples of sugar alcohol includes: sorbitol, xylitol, maltitol, mannitol, or combinations thereof

A pharmaceutical composition disclosed herein (e.g., comprising a Cas9 protein described herein) can be formulated for any route of administration to a subject. Specific examples of routes of administration include intramuscularly, cutaneously, subcutaneously, ophthalmic, intravenously, intraperitoneally, intradermally, intraorbitally, intracerebrally, intracranially, intraspinally, intraventricular, intrathecally, intracapsularly, orally, rectally, vaginally, or intratumorally or via intratympanic injection. Parenteral administration, characterized by, e.g., cutaneous, subcutaneous, intramuscular, or intravenous injection, is also contemplated herein.

Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

A therapeutic agent described herein (e.g., Cas9 protein variant having enhanced specificity) can be formulated as an aerosol for topical application, such as by inhalation (see, e.g., U.S. Pat. Nos. 4,044,126; 4,414,209; and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation can have diameters of less than about 50 microns, e.g., less than about 10 microns.

A therapeutic agent disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies.

Transdermal patches, e.g., iontophoretic and electrophoretic devices, are known to those of skill in the art, and can be used to administer a therapeutic agent (e.g., those disclosed herein). For example, such patches are disclosed in U.S. Pat. Nos. 6,267,983; 6,261,595; 6,256,533; 6,167,301; 6,024,975; 6,010,715; 5,985,317; 5,983,134; 5,948,433; and 5,860,957.

In some aspects, a pharmaceutical composition described herein is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, e.g., citrate, sodium or potassium phosphate or other such buffer known to those of skill in the art at, in some aspects, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Pharmaceutical compositions provided herein can also be formulated to be targeted to a particular tissue, receptor, or other area of the body of the subject to be treated. Many such targeting methods are known to those of skill in the art. All such targeting methods are contemplated herein for use in the instant compositions. For non-limiting examples of targeting methods, see, e.g., U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

Pharmaceutical compositions to be used for in vivo administration can be sterile. This can be accomplished, for example, by filtration through, e.g., sterile filtration membranes.

### V. Kits/Systems

Also disclosed herein are kits comprising any of the Cas9 proteins, polynucleotides, vectors, compositions, or cells described herein. In some aspects, the kit includes one or more containers comprising any of the Cas9 proteins, polynucleotides, vectors, compositions, or cells described herein. In some aspects, the kit further comprises instructions for use, e.g., in accordance with any of the methods provided herein.

One skilled in the art will readily recognize that any of the Cas9 proteins, polynucleotides, vectors, compositions, or cells described herein can be readily incorporated into one of the established kit formats which are well known in the art. In some aspects, the kits further comprise additional components such as buffers and interpretive information. In some aspects, the kit comprises a container and a label or package insert(s) on or associated with the container. In some aspects, the disclosure provides articles of manufacture comprising the contents of the kits described herein.

The present disclosure further provides a gene editing system, comprising: (i) any of the Cas9 proteins described herein, and (ii) a guide polynucleotide. In some aspects, the guide polynucleotide is a guide RNA, which comprises a guide sequence that is complementary to a target sequence of a gene to be modified. As is apparent from the present disclosure, compared to other systems available in the art, the gene editing system of the present disclosure allows for reduced off-target effects and/or increased on-target effects during the gene editing process.

As described herein, in some aspects, the likelihood of an off-target effect (e.g., cleavage at a non-target sequence) is reduced by at least about 1-fold, at least about 2-fold, at least about 3-fold, at least about 4-fold, at least about 5-fold, at least about 10-fold, at least about 15-fold, least about 20-fold, at least about 25-fold, at least about 30-fold, at least about 40-fold, or at least about 50-fold or more, compared to the off-target effects observed with other gene editing systems in the art (e.g., using a wild-type Cas9 protein).

### VI. Methods of the Disclosure

VIA. Methods of Making

Also encompassed by the present disclosure is a method for producing/making a Cas9 protein described herein. In some aspects, such a method can comprise expressing Cas9 protein in a cell comprising a nucleic acid molecule encoding the protein. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, bacterial cell, insect cell, plant cell, yeast cell, or combinations thereof.

Related to the above method of producing a Cas9 protein, the present disclosure is also directed to methods producing/making the Cas9 proteins described herein. Specifically, provided herein is a method of increasing the specificity of a Cas9 protein, such that the Cas9 protein is capable of more accurately recognizing one or more base mismatches within a gene sequence (e.g., within the target sequence and/or the PAM). Applicant has discovered that modifying certain amino acid residues within the cavity domain of a Cas9 protein can increase the specificity of the Cas9 protein. In some aspects, one or more of the amino acid residues that are modified is capable of interacting with a backbone phosphate of a DNA sequence. Not to be bound by any one theory, in some aspects, such a modification modulates the interaction between the Cas9 protein and a nucleic acid sequence (e.g., does not bind as tightly), such that the Cas9 protein does not cleave the nucleic acid sequence if it comprises one or more base mismatches.

Non-limiting examples of amino acid residues that can be modified are provided elsewhere in the present disclosure. Additionally, methods for introducing amino acid modifications (e.g., substitutions) into an amino acid sequence of a polypeptide are available in the art. Nucleic acids encoding variant nucleases can be introduced into a viral or a non-viral vector for expression in a host cells (e.g., human cell, animal cell, bacterial cell, yeast cell, insect cell). In some aspects, nucleic acids encoding variant nucleases are operably linked to one or more regulatory domains for expression of the nuclease. Suitable bacterial and eukaryotic promoters are well known in the art and described in e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Paiva et al., 1983, Gene 22:229-235).

VI.B. Diagnostic Uses

The present disclosure is also directed to methods of determining the expression level of a biomarker (e.g., a nucleotide sequence), such as that associated with a disease or disorder (e.g., cancer and/or neurodegenerative diseases). As is apparent to those skilled in the arts, in some aspects, determining the expression level of a biomarker allows the diagnosis of a disease or disorder in a subject in need thereof. Therefore, in some aspects, disclosures provided herein is directed to a method of diagnosing a disease or disorder in a subject. In some aspects, such a method comprises determining the expression level of a biomarker, which is associated with a disease or disorder, in a subject, the method comprising contacting a biological sample obtained from the subject with a Cas9 protein described herein (e.g., modified to exhibit enhanced selectivity). As described herein, in some aspects, the Cas9 protein is contacted with the biological sample in combination with a guide RNA. In some aspects, the diagnostic methods provided herein further comprises determining the expression level of the biomarker in the biological sample.

In some aspects, an abnormal level (increased or decreased) of the biomarker in the biological sample indicates that the subject has or is at risk of developing the disease or disorder. In some aspects, the expression level of the biomarker in the biological sample is increased compared to a corresponding expression level in a reference sample (e.g., biological sample obtained from a subject determined not to have nor at risk of developing the disease or disorder). In some of such aspects, the expression level of the biomarker in the biological sample is at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold or more higher than the corresponding expression level in the reference sample. In some aspects, the expression level of the biomarker in the biological sample is decreased compared to a corresponding expression level in a reference sample (e.g., biological sample obtained from a subject determined not to have nor at risk of developing the disease or disorder). In some of such aspects, the expression level of the biomarker in the biological sample is at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold or more less than the corresponding expression level in the reference sample.

As described herein and also apparent to those skilled in the arts, for many diseases and disorders, the amount of the biomarker present in the subject can be extremely low. For example, the amount of circulating tumor DNA (ctDNA) that is present in the blood of a cancer patient can be as low as 0.01% of the total cell-free DNA, and thus, making the detection of such a biomarker very difficult. See, e.g., Elazezy et al., Comput Struct Biotechnol J 16: 370-378 (October 2018); Schwarzenbach et al., Ann N Y Acad Sci 1137: 190-6 (August 2008); Forshew et al., Sci Transl Med 4(136): 136ra68 (May 2012); and Kennedy et al., Nat Protoc 9(11): 2586-606 (November 2014); each of which is incorporated herein by reference in its entirety.

Compared to methods available in the art, the diagnostic methods provided herein allow for more accurate and cost-efficient approach to measuring a biomarker, including those that are present at very low frequency in subjects afflicted with a disease or disorder. Not to be bound by any one theory, in some aspects, the diagnostic methods provided herein are superior to those available in the art, as the contacting of the biological sample with a Cas9 protein described herein reduces the amount of one or more polynucleotides that differ (e.g., in sequence) from the biomarker, such that the biological sample is enriched for the biomarker.

Accordingly, provided herein is a method of measuring a first nucleotide sequence (i.e., biomarker) in a biological sample comprising the first nucleotide sequence and a second nucleotide sequence, wherein the first nucleotide sequence and the second nucleotide sequence are not the same, the method comprising contacting the biological sample with any of the Cas9 proteins described herein, wherein the contacting reduces the amount of the second nucleotide sequence present in the biological sample. In some aspects, the first nucleotide sequence is a biomarker for a disease or disorder (e.g., comprises a mutation associated with the disease or disorder). In some aspects, the Cas9 protein is contacted with the biological sample in combination with a guide RNA. Not to be bound by any one theory, by reducing the amount of the second nucleotide sequence present in the biological sample, in some aspects, the presence of the biomarker (i.e., the first nucleotide sequence) can be more accurately measured.

In some aspects, the first and second nucleotide sequences differ only as to the specific mutation (i.e., base mismatch compared to the guide sequence of the gRNA) present in the first nucleotide sequence. In some aspects, the mutation present in the first nucleotide sequence comprises a substitution, insertion, deletion, deletion-insertion (indel), duplication, inversion, large genomic rearrangement, or a combination thereof. In some aspects, the mutation comprises a single nucleotide. In some aspects, the mutation comprises multiple nucleotides (e.g., at least about two, at least about three, at least about four, at least about five, at least about six, at least about seven, at least about eight, at least about nine, or at least about 10 or more nucleotides). In some aspects, a mutation is within the target sequence to which the Cas9 protein (e.g., Cas9:gRNA complex) binds to. In some aspects, a mutation is within the PAM. In some aspects, a mutation is within both the target sequence and the PAM.

As is apparent from the present disclosure, when the biological sample is contacted with the Cas9 protein, the Cas9 protein is capable of recognizing the mutation present within the first nucleotide sequence. Accordingly, the Cas9 protein does not cleave the first nucleotide sequence but does cleave the second nucleotide sequence, which comprises a target sequence that is complementary to the guide sequence of the gRNA. As a result, after the contacting, the amount of the second nucleotide sequence present in the biological sample is reduced by: at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

In some aspects, by reducing the amount of the second nucleotide sequence present in a biological sample, it is possible to enrich for the first nucleotide sequence comprising the mutation (i.e., biomarker) in the biological sample. Accordingly, in some aspects, provided herein is a method of enriching for a first nucleotide sequence in a biological sample comprising the first nucleotide sequence and a second nucleotide sequence, wherein the first nucleotide sequence and the second nucleotide sequence are not the same, the method comprising contacting the biological sample with any of the Cas9 proteins described herein (e.g., modified to exhibit enhanced specificity), wherein after the contacting, the biological sample comprises a greater percentage of the first nucleotide sequence. In some aspects, the Cas9 protein is contacted with the biological sample in combination with a guide RNA. Not to be bound by any one theory, by enriching the biological sample for the first nucleotide sequence (i.e., the first nucleotide sequence makes up a greater percentage of the biological sample), in some aspects, the presence of the biomarker (i.e., the first nucleotide sequence) can be more accurately measured.

In some aspects, after the contacting, the percentage of the first nucleotide sequence present in the biomarker is increased by at least about 1-fold, at least about 2-fold, at least about 3-fold, at least about 4-fold, at least about 5-fold, at least about 10-fold, at least about 15-fold, least about 20-fold, at least about 25-fold, at least about 30-fold, at least about 40-fold, or at least about 50-fold or more compared to the corresponding percentage in a reference sample (e.g., the biological sample prior to the contacting).

In some aspects, the diagnosing is performed ex vivo. For example, the contacting of the biological sample with a Cas9 protein described herein can occur in vitro. In some aspects, both the contacting and determining the expression level of the biomarker occurs ex vivo.

As is apparent from the present disclosure, the expression level of the biomarker (e.g., first nucleotide sequence comprising a mutation associated with a disease or disorder) can be determined using any suitable methods known in the art. For example, the expression level of the biomarker can be determined using any sequencing-based methods described herein (see, e.g., Example 1) and/or known in the art (e.g., PCR, real-time PCR, microarray, next-generation sequencing (NGS), Sanger sequencing, LAMP, RFLP).

As described herein, in some aspects, the diagnostic methods provided herein comprise contacting a biological sample obtained from the subject with a Cas9 protein of the present disclosure (e.g., in combination with a guide RNA). As used herein, the term “biological sample” refers to any sample that contains a material that can be derived from a subject (e.g., human). Non-limiting examples of biological samples useful for the present disclosure include: a tissue, blood, cerebrospinal fluid (CSF), amniotic fluid, semen, vaginal fluid, urine, saliva, sputum, rhinorrhea, tears, sweat, stool, horny substance, hair, bile juice, pancreatic juice, gastric juice, serous fluid, transudate, synovial fluid, exudate, abscess, interstitial fluid (ISF), serum, plasma, cell culture media, or any combination thereof. In some aspects, a biological sample comprises blood. In some aspects, a biological sample comprises CSF. In some aspects, a biological sample comprises serum. In some aspects, a biological sample comprises plasma. In some aspects, a biological sample comprises cell culture media. In some aspects, a biological sample comprises both blood and CSF. In some aspects, a biological sample comprises any combination of blood, CSF, serum, plasma, and culture media.

In some aspects, once a subject has been diagnosed as suffering from or being at a high risk of developing a disease or disorder, the subject can be treated with a therapy, which, e.g., helps reduce or eliminate one or more symptoms of the disease (“therapeutic treatment”) or prevents or delays the onset of the disease (“prophylactic treatment”). Accordingly, in some aspects, the diagnostic methods provided herein further comprises administering a treatment/therapy to a subject identified as having the disease or at risk of developing the disease using the methods provided herein. Additional disclosures relating to such treatments are provided elsewhere in the present disclosure.

Additionally, as described herein, the diagnostic methods provided herein can be useful in diagnosing a wide range of diseases and conditions. It will be apparent to those skilled in the arts that as long as the specific disease or disorder is associated with a certain biomarker (e.g., unique DNA pattern resulting from the specific mutation present within the gene), the Cas9 protein and its accompany gRNA can be modified to identify the particular biomarker. Non-limiting examples of such diseases or conditions are provided elsewhere in the present disclosure. For example, in some aspects, diseases or conditions that are applicable for the present disclosure include: oncologic diseases (e.g., malignant tumor/benign tumor), hematologic diseases (e.g., leukemia/lymphoma), neurodegenerative diseases (e.g., Alzheimer's disease/Parkinson's disease), infectious diseases (e.g., viral infection/bacterial infection), rheumatoid diseases (e.g., rheumatoid arthritis/ankylosing spondylitis), neurologic diseases (e.g., stroke/amyotrophic lateral sclerosis), allergic diseases (e.g., dermatitis/asthma), psychiatric diseases (e.g., schizophrenia/depression), optical diseases (e.g., keratitis/retinitis), endocrinologic diseases (e.g., diabetes mellitus/thyroid insufficiency), congenital diseases (e.g., Down syndrome/neurofibromatosis), obstetric diagnosis (e.g., prenatal diagnosis/pregnancy diagnosis), cardiovascular diseases (e.g., myocardial infarction/arrhythmia), pulmonary diseases (e.g., pulmonary embolism/bronchitis), nephrologic diseases (e.g., nephritis/renal injury), gastrologic diseases (e.g., gastritis/reflux disease), hepatologic diseases (e.g., hepatitis/liver cirrhosis), and combinations thereof.

VI.C. Therapeutic Uses

As is apparent from the present disclosure, the Cas9 proteins described herein (e.g., modified to exhibit enhanced specificity) can be useful in a wide range of clinical settings, in addition to the above-described diagnostic methods. With advancements of gene-editing technologies (e.g., CRISPR-Cas9 system), it is becoming more possible to treat various diseases and disorders through gene modification (e.g., gene therapy). For example, by regulating the expression of a gene (e.g., deleting a mutated gene and/or introducing a healthy gene), the function of a cell can be restored and/or improved, and thereby treat the disease or disorder.

Accordingly, in some aspects, provided herein is a method of genetically modifying a cell, comprising contacting the cell with any of the Cas9 proteins provided herein (e.g., modified to exhibit enhanced specificity), wherein after the contacting the expression and/or activity of one or more genes in the cell is modified. In some aspects, the Cas9 protein is contacted with the cell in combination with a guide RNA, wherein the guide RNA comprises a guide sequence that is complementary (e.g., fully complementary) to a target sequence within a gene or genes to be modified. Once modified, such cells can be administered to a subject, such that the administered cells can provide the necessary function aberrant in the subject. In some aspects, the modified cells are derived from the subject to be treated. In some aspects, prior to the contacting, the cell is isolated from the subject, and then after the contacting, the modified cell is reintroduced to the subject. In some aspects, the cell that is contacted with the Cas9 protein is from a donor (e.g., healthy donor).

In some aspects, prior to the contacting, the subject to be treated receives an administration of the Cas9 proteins described herein, such that the contacting and the modifying occur in vivo. Suitable methods of administration are provided elsewhere in the present disclosure.

In some aspects, after the modification, the expression and/or activity of the one or more genes is increased by at least about 1-fold, at least about 2-fold, at least about 3-fold, at least about 4-fold, at least about 5-fold, at least about 10-fold, at least about 15-fold, least about 20-fold, at least about 25-fold, at least about 30-fold, at least about 40-fold, or at least about 50-fold or more compared to the expression and/or activity of the one or more genes in a reference cell (e.g., the cell prior to the contacting and modification). In some aspects, after the modification, the expression and/or activity of the one or more genes is decreased by at least about 1-fold, at least about 2-fold, at least about 3-fold, at least about 4-fold, at least about 5-fold, at least about 10-fold, at least about 15-fold, least about 20-fold, at least about 25-fold, at least about 30-fold, at least about 40-fold, or at least about 50-fold or more compared to the expression and/or activity of the one or more genes in a reference cell (e.g., the cell prior to the contacting and modification).

In some aspects, the gene to be modified differs (e.g., in sequence) from other genes present in the cell (“reference gene”). For example, in some aspects, the nucleic acid sequence of the gene to be modified has a sequence identity that is less than about 99%, less than about 98%, less than about 97%, less than about 96%, less than about 95%, less than about 94%, less than about 93%, less than about 92%, less than about 91%, less than about 90%, less than about 85%, less than about 80%, or less than about 75% to the nucleic acid sequence of the reference gene. In some aspects, both the gene to be modified and the reference gene comprises a target sequence, PAM or both. In some aspects, the target sequence of the gene to be modified differs from the target sequence of the reference gene by one or more nucleotides. In some aspects, the PAM of the gene to be modified differs from the PAM of the reference gene by one or more nucleotides. In some aspects, both the target sequence and the PAM of the gene to be modified differ from those of the reference gene by one or more nucleotides.

As described herein, the Cas9 proteins of the present disclosure are capable of recognizing even a single base mismatch within a target sequence and/or the PAM, such that the Cas9 protein does not cleave a gene comprising such a base mismatch. Because of such enhanced specificity, the Cas9 proteins described herein allow for increased on-target effects and/or decreased off-target effects.

Provided herein is also a method of increasing the on-target effects during CRISPR-based gene editing of a cell, comprising contacting the cell with a Cas9 protein that has been modified to comprise one or more amino acid modifications, which increase the specificity of the Cas9 protein. In some aspects, the Cas9 protein comprises any of the modified Cas9 proteins described herein. The Cas9 protein can be contacted with the cell in combination with a guide RNA. In some aspects, the on-target effects are increased by at least about 1-fold, at least about 2-fold, at least about 3-fold, at least about 4-fold, at least about 5-fold, at least about 10-fold, at least about 15-fold, least about 20-fold, at least about 25-fold, at least about 30-fold, at least about 40-fold, or at least about 50-fold or more, compared to the on-target effects observed during a CRISPR-based gene editing of a cell using a reference Cas9 protein (e.g., wild-type Cas9 protein).

Similarly, in some aspects, provided herein is a method of reducing the occurrence of an off-target effect (e.g., cleavage at a non-target sequence) during a CRISPR-based gene editing of a cell, comprising contacting the cell with a Cas9 protein that has been modified to comprise one or more amino acid modifications, which increase the specificity of the Cas9 protein. In some aspects, the Cas9 protein comprises any of the modified Cas9 proteins described herein. In some aspects, the Cas9 protein is contacted with the cell in combination with a guide RNA. In some aspects, the occurrence of an off-target effect is reduced by at least about 1-fold, at least about 2-fold, at least about 3-fold, at least about 4-fold, at least about 5-fold, at least about 10-fold, at least about 15-fold, least about 20-fold, at least about 25-fold, at least about 30-fold, at least about 40-fold, or at least about 50-fold or more, compared to the off-target effects observed during a CRISPR-based gene editing of a cell using a reference Cas9 protein (e.g., wild-type Cas9 protein).

As demonstrated, with the Cas9 proteins of the present disclosure, there is less than about 70, less than about 65, less than about 60, less than about 55, less than about 50, less than about 45, less than about 40, less than about 35, less than about 30, less than about 25, less than about 20, less than about 15, less than about 10, of less than about 5 off-target site cleavages observed, e.g., as measured using Digenome-seq analysis. In some aspects, with the Cas9 proteins described herein, a single off-target site cleavage can occur. In some aspects, with the Cas9 proteins described herein, no off-target site cleavages occur during a CRISPR-based gene editing of a cell.

As described herein, the present disclosure can be used to treat any suitable diseases or disorders known in the art. Non-limiting examples of such diseases and disorders are provided elsewhere in the present disclosure.

In some aspects, a therapeutic method provided herein further comprises administering one or more additional agents to the subject. For example, where the subject suffers from a cancer, the additional agent can comprise an anti-cancer agent. Non-limiting examples of such anti-cancer agents include chemotherapy, immunotherapy (e.g., checkpoint inhibitors), or both. Where the subject suffers from a neurodegenerative disease, the additional therapeutic agent comprises an acetylcholinesterase inhibitor. In some aspects, the additional therapeutic agent comprises a dopamine agonist. In some aspects, the additional therapeutic agent comprises a dopamine receptor antagonist. In some aspects, the additional therapeutic agent comprises an antipsychotic. In some aspects, the additional therapeutic agent comprises a monoamine oxidase (MAO) inhibitor. In some aspects, the additional therapeutic agent comprises a catechol O-methyltransferase (COMT) inhibitor. In some aspects, the additional therapeutic agent comprises a N-methyl-D-aspartate (NMDA) receptor antagonist. In some aspects, the additional therapeutic agent comprises an immunomodulatory. In some aspects, the additional therapeutic agent comprises an immunosuppressant.

The following examples are merely illustrative and should not be construed as limiting the scope of this disclosure in any way as many variations and equivalents will become apparent to those skilled in the art upon reading the present disclosure.

### EXAMPLES

### Example 1: Materials and Methods

The following examples used one or more of the materials and methods described below.

### Protein Engineering (Structural Analysis) and Cloning

Protein structure of FnCas9 (PDB ID 5B2O) was analyzed by Pymol. And residues of FnCas9 making hydrogen bond distance with DNA were marked with sphere. Those residues were changed to alanine using QuikChange II Site-Directed Mutagenesis Kit (Agilent). Briefly, wild-type FnCas9 (pET28-a) was used as template to amplify FnCas9 variants using primers containing alanine point mutation. The FnCas9 variants was cloned according to manufacturer's instrument, which has Hisx6 tag at N-terminal end of the recombinant FnCas9 protein.

### Protein Purification

pET-a vector containing FnCas9 variant under T7 promoter was transformed into BL21-DE competent cell (Novagen) according to manufacturer's instrument. Cell harboring pET-FnCas9 variant was cultured in LB media (Duchefa, Haarlem, The Netherlands) at 37° C. IPTG(Beams bio) was treated when value of OD600 nm reach at range of 0.5˜0.7. Cell was harvested after an overnight incubation at 18° C. Cell was lysed in lysis buffer (50 mM NaH2PO4, 300 mM NaCl, 10 mM Imidazole, 1 mg/ml Lysozyme, 1 mM PMSF, 1 mM DTT, pH 8) using ultra-sonicator. Cell lysate was centrifuged at 15000 rpm to remove cell debris. Clear supernatant containing FnCas9 variant proteins was treated with Ni NTA bead (Qiagen). Ni NTA bead with FnCas9 protein was washed with a wash buffer (50 mM NaH2PO4, 300 mM NaCl, 20 mM Imidazole, pH 8). Protein was eluted with a elution buffer (50 mM NaH2PO4, 300 mM NaCl, 250 mM Imidazole, pH 8) and maintained in a storage buffer (50 mM HEPES, 200 mM NaCl, 20% Glycerol, 1 mM DTT, pH 7.5) until further analysis.

In Vitro Transcription of sgRNA

Single guide RNAs (sgRNA) of SpCas9 and FnCas9 were designed and synthesized with single base pair mutation. The sgRNAs were synthesized by in vitro transcription. Briefly, RNA template was transcribed using T7 RNA polymerase in 40 mM Tris-HCl (pH 7.9), 6 mM MgCl₂, 10 mM DTT, 10 mM NaCl, 2 mM spermidine, NTPs, and a RNase inhibitor. The reaction mixture was incubated at 37° C. for 8 hours, and the transcribed sgRNAs were purified using a PCR purification kit (GeneAll, Seoul, Korea) and quantified using a NanoDrop spectrophotometer.

### In Vitro DNA Cleavage Assay

A 3 kb target DNA including KRAS, NRAS, EGFR gene sequence was cleaved with Cas9 proteins and sgRNAs. (Table 3). KRAS, NRAS, and EGFR target site are synthesized by IDT, and cloned into p3 vector. A 3 kb target DNA was amplified by PCR from p3 vector including target site, two pairs of primer and Q5 DNA polymerase (New England Biolabs). Reactions were cleaned up with PCR clean-up kit (GeneA11). The DNA template was incubated with guide RNA, variants of Cas9 in Cutsmart buffer (New England Biolabs) (100 mM Potassium acetate, 20 mM Tris-acetate, 10 mM Magnesium acetate, 100 ug/ml BSA, pH 7.9) for 1 h at 37° C. To analyze cleavage by nuclease, digested DNA fragments were run on TBE 1.5% agarose gels followed by ethidium bromide staining.

TABLE 3
KRAS, NRAS, and EGFR DNA Template Sequences

| Gene | Sequence |
|---|---|
| KRAS_3Kb | gggaataagggcgacacggaaatgttgaatactcatactcttcctttttcaatattatt |
| (SEQ ID NO: 281) | gaagcatttatcagggttattgtctcatgageggatacatatttgaatgtatttagaaa |
| | aataaacaaataggggttccgcgcacatttccccgaaaagtgccacctgacgtcgacgg |
| | atcgggagatctcccgatcccctatggtcgactctcagtacaatctgctctgatgccgc |
| | atagttaagccagtatctgctccctgcttgtgtgttggaggtcgctgagtagtgcgcga |
| | gcaaaatttaagctacaacaaggcaaggcttgaccgacaattgcatgaagaatctgctt |
| | agggttaggcgttttgcgctgcttcgcgatgtacgggccagatatacgcgttgacattg |
| | attattgactagttattaatagtaatcaattacggggtcattagttcatagcccatata |
| | tggagttccgcgttacataacttacggtaaatggcccgcctggctgaccgcccaacgac |
| | ccccgcccattgacgtcaataatgacgtatgttcccatagtaacgccaatagggacttt |
| | ccattgacgtcaatgggtggactatttacggtaaactgcccacttggcagtacatcaag |
| | tgtatcatatgccaagtacgccccctattgacgtcaatgacggtaaatggcccgcctgg |
| | cattatgcccagtacatgaccttatgggactttcctacttggcagtacatctacgtatt |
| | agtcatcgctattaccatggtgatgcggttttggcagtacatcaatgggcgtggatagc |
| | ggtttgactcacggggatttccaagtctccaccccattgacgtcaatgggagtttgttt |
| | tggcaccaaaatcaacgggactttccaaaatgtcgtaacaactccgccccattgacgca |
| | aatgggcggtaggcgtgtacggtgggaggtctatataagcagagctctctggctaacta |
| | gagaacccactgcttactggcttatcgaaattaatacgactcactatagggagacccAA |
| | GCTTATGATTCTGAATTAGCTGTATCGTCAAGGCACTCTTGCCTacgccaccagctcca |
| | actacCACAAGTTTATATTCAGTCATTTTCAGCAGGCCTTATAATtctagagggcccta |
| | ttctatagtgtcacctaaatgctagagctcgctgatcagcctcgactgtgccttctagt |
| | tgccagccatctgttgtttgcccctcccccgtgccttccttgaccctggaaggtgccac |
| | tcccactgtectttcctaataaaatgaggaaattgcatcgcattgtctgagtaggtgtc |
| | attctattctggggggtggggtggggcaggacagcaagggggaggattgggaagacaat |
| | agcaggcatgctggggatgcggtgggctctatggcttctgaggcggaaagaaccagctg |
| | cattaatgaatcggccaacgcgcggggagaggcggtttgcgtattgggcgctcttccgc |
| | actcaaaggcggtaatacggttatccacagaatcaggggataacgcaggaaagaacatg |
| | tgagcaaaaggccagcaaaaggccaggaaccgtaaaaaggccgcgttgctggcgttttt |
| | ccataggetccgcccccctgacgagcatcacaaaaatcgacgctcaagtcagaggtggc |
| | gaaacccgacaggactataaagataccaggcgtttccccctggaagctccctcgtgcgc |
| | tctcctgttccgaccctgccgcttaccggatacctgtccgcctttctcccttcgggaag |
| | cgtggcgctttctcaatgctcacgctgtaggtatctcagttcggtgtaggtcgttcgct |
| | ccaagctgggctgtgtgcacgaaccccccgttcagcccgaccgctgcgccttatccggt |
| | aactatcgtcttgagtccaacccggtaagacacgacttatcgccactggcagcagccac |
| | tggtaacaggattagcagagcgaggtatgtaggcggtgctacagagttcttgaagtggt |
| | ggcctaactacggctacactagaaggacagtatttggtatctgcgctctgctgaagcca |
| | gttaccttcggaaaaagagttggtagctcttgatccggcaaacaaaccaccgctggtag |
| | cggtggtttttttgtttgcaagcagcagattacgcgcagaaaaaaaggatctcaagaag |
| | atcctttgatcttttctacggggtctgacgctcagtggaacgaaaactcacgttaaggg |
| | attttggtcatgagattatcaaaaaggatcttcacctagatccttttaaattaaaaatg |
| | aagttttaaatcaatctaaagtatatatgagtaaacttggtctgacagttaccaatgct |
| | taatcagtgaggcacctatctcagcgatctgtctatttcgttcatccatagttgcctga |
| | ctccccgtcgtgtagataactacgatacgggagggcttaccatctggccccagtgctgc |
| | aatgataccgcgagacccacgctcaccggctccagatttatcagcaataaaccagccag |
| | ccggaagggccgagcgcagaagtggtcctgcaactttatccgcctcatccagtctatt |
| | aattgttgccgggaagctagagtaagtagttcgccagttaatagtttgcgcaacgttgt |
| | tgccattgctacaggcatcgtggtgtcacgctcgtcgtttggtatggcttcattcagct |
| | ccggttcccaacgatcaaggcgagttacatgatcccccatgttgtgcaaaaaagcggtt |
| | agctccttcggtcctccgatcgttgtcagaagtaagt |
| NRAS_3kb | gggaataagggcgacacggaaatgttgaatactcatactcttcctttttcaatattatt |
| (SEQ ID NO: 282) | gaagcatttatcagggttattgtctcatgageggatacatatttgaatgtatttagaaa |
| | aataaacaaataggggttccgcgcacatttccccgaaaagtgccacctgacgtcgacgg |
| | atcgggagatctcccgatcccctatggtcgactctcagtacaatctgctctgatgccgc |
| | atagttaagccagtatctgctccctgcttgtgtgttggaggtcgctgagtagtgcgcga |
| | gcaaaatttaagctacaacaaggcaaggcttgaccgacaattgcatgaagaatctgctt |
| | agggttaggcgttttgcgctgcttcgcgatgtacgggccagatatacgcgttgacattg |
| | attattgactagttattaatagtaatcaattacggggtcattagttcatagcccatata |
| | tggagttccgcgttacataacttacggtaaatggcccgcctggctgaccgcccaacgac |
| | ccccgcccattgacgtcaataatgacgtatgttcccatagtaacgccaatagggacttt |
| | ccattgacgtcaatgggtggactatttacggtaaactgcccacttggcagtacatcaag |
| | tgtatcatatgccaagtacgccccctattgacgtcaatgacggtaaatggcccgcctgg |
| | cattatgcccagtacatgaccttatgggactttcctacttggcagtacatctacgtatt |
| | agtcatcgctattaccatggtgatgcggttttggcagtacatcaatgggcgtggatagc |
| | ggtttgactcacggggatttccaagtctccaccccattgacgtcaatgggagtttgttt |
| | tggcaccaaaatcaacgggactttccaaaatgtcgtaacaactccgccccattgacgca |
| | aatgggcggtaggcgtgtacggtgggaggtctatataagcagagctctctggctactag |
| | agaacccactgcttactggcttatcgaaattaatacgactcactatagggagacccaag |
| | cttTCGCCTAGTCCTCATGTATTGGTCTCTCATGGCACTGTCCTCTTCTTGTCCAgctg |
| | tatccagtatgtccaaCAAACAGGTTTCACCATCTATAACCACTTtctagagggcccta |
| | ttctatagtgtcacctaaatgctagagctcgctgatcagcctcgactgtgccttctagt |
| | tgccagccatctgttgtttgcccctcccccgtgccttccttgaccctggaaggtgccac |
| | teccactgtcctttcctaataaaatgaggaaattgcatcgcattgtctgagtaggtgtc |
| | attctattctggggggtggggtggggcaggacagaagggggagggattgggaagacaat |
| | agcaggcatgctggggatgcggtgggctctatggcttctgaggcggaaagaaccagctg |
| | cattaatgaatcggccaacgcgcggggagaggcggtttgcgtattgggcgctcttccgc |
| | ttcctcgctcactgactcgctgcgctcggtcgttcggctgcggcgagcggtatcagctc |
| | actcaaaggcggtaatacggttatccacagaatcaggggataacgcaggaaagaacatg |
| | tgagcaaaaggccagcaaaaggccaggaaccgtaaaaaggccgcgttgctggcgttttt |
| | ccataggetccgcccccctgacgagcatcacaaaaatcgacgctcaagtcagaggtggc |
| | gaaacccgacaggactataaagataccaggcgtttccccctggaagctccctcgtgcgc |
| | tctcctgttccgaccctgccgcttaccggatacctgtccgcctttctcccttcgggaag |
| | cgtggcgctttctcaatgctcacgctgtaggtatctcagttcggtgtaggtcgttcgct |
| | ccaagctgggctgtgtgcacgaaccccccgttcagcccgaccgctgcgccttatccggt |
| | aactatcgtcttgagtccaacccggtaagacacgacttatcgccactggcagcagccac |
| | tggtaacaggattagcagagcgaggtatgtaggcggtgctacagagttcttgaagtggt |
| | ggectaactacggctacactagaaggacagtatttggtatctgcgctctgctgaagcca |
| | gttaccttcggaaaaagagttggtagctcttgatccggcaaacaaaccaccgctggtag |
| | cggtggtttttttgtttgcaagcagcagattacgcgcagaaaaaaaggatctcaagaag |
| | atcctttgatcttttctacggggtctgacgctcagtggaacgaaaactcacgttaaggg |
| | attttggtcatgagattatcaaaaaggatcttcacctagatccttttaaattaaaaatg |
| | aagttttaaatcaatctaaagtatatatgagtaaacttggtctgacagttaccaatgct |
| | taatcagtgaggcacctatctcagcgatctgtctatttcgttcatccatagttgcctga |
| | ctccccgtcgtgtagataactacgatacgggagggcttaccatctggccccagtgctgc |
| | aatgataccgcgagacccacgctcaccggetccagatttatcagcaataaaccagccag |
| | ccggaagggccgagcgcagaagtggtcctgcaactttatccgcctccatccagtctatt |
| | aattgttgccgggaagctagagtaagtagttcgccagttaatagtttgcgcaacgttgt |
| | tgccattgctacaggcatcgtggtgtcacgctcgtcgtttggtatggcttcattcagct |
| | ccggttcccaacgatcaaggcgagttacatgatcccccatgttgtgcaaaaaagcggtt |
| | agctcctteggtcctccgatcgttgtcagaagtaagt |
| EGFR_3kb | gggaataagggcgacacggaaatgttgaatactcatactcttcctttttcaatattatt |
| (SEQ ID NO: 283) | gaagcatttatcagggttattgtctcatgagcggatacatatttgaatgtatttagaaa |
| | aataaacaaataggggttccgcgcacatttccccgaaaagtgccacctgacgtcgacgg |
| | atcgggagatctcccgatcccctatggtcgactctcagtacaatctgctctgatgccgc |
| | atagttaagccagtatctgctccctgcttgtgtgttggaggtcgctgagtagtgcgcga |
| | gcaaaatttaagctacaacaaggcaaggcttgaccgacaattgcatgaagaatctgctt |
| | agggttaggcgttttgcgctgcttcgcgatgtacgggccagatatacgcgttgacattg |
| | attattgactagttattaatagtaatcaattacggggtcattagttcatagcccatata |
| | tggagttccgcgttacataacttacggtaaatggcccgcctggctgaccgcccaacgac |
| | ccattgacgtcaatgggtggactatttacggtaaactgcccacttggcagtacatcaag |
| | tgtatcatatgccaagtacgccccctattgacgtcaatgacggtaaatggcccgcctgg |
| | cattatgcccagtacatgaccttatgggactttcctacttggcagtacatctacgtatt |
| | agtcatcgctattaccatggtgatgcggttttggcagtacatcaatgggcgtggatagc |
| | ggtttgactcacggggatttccaagtctccaccccattgacgtcaatgggagtttgttt |
| | tggcaccaaaatcaacgggactttccaaaatgtcgtaacaactccgccccattgacgca |
| | aatgggcggtaggcgtgtacggtgggaggtctatataagcagagctctctggctaacta |
| | gagaacccactgcttactggcttatcgaaattaatacgactcactatagggagacccaa |
| | gcttCTTGGTGCACCGCGACCTGGCAGCCAGGAACGTACTGGTGAAAACACCGCAGCAT |
| | GTCAAGATCAcagattttgggctggccaaaCGGCTGGGTGCGGAAGAGAAAGAtctaga |
| | gggccctattctatagtgtcacctaaatgctagagctcgctgatcagcctcgactgtgc |
| | cttctagttgccagccatctgttgtttgcccctcccccgtgccttccttgaccctggaa |
| | ggtgccactcccactgtcctttcctaataaaatgaggaaattgcatcgcattgtctgag |
| | taggtgtcattctattctggggggtggggtggggcaggacagcaagggggaggattggg |
| | aagacaatagcaggcatgctggggatgcggtgggctctatggcttctgaggcggaaaga |
| | accagctgcattaatgaatcggccaacgcgcggggagaggcggtttgcgtattgggcgc |
| | atcagctcactcaaaggcggtaatacggttatccacagaatcaggggataacgcaggaa |
| | agaacatgtgagcaaaaggccagcaaaaggccaggaaccgtaaaaaggccgcgttgctg |
| | gegtttttccataggctccgcccccctgacgagcatcacaaaaatcgacgctcaagtca |
| | gaggtggcgaaacccgacaggactataaagataccaggcgtttccccctggaagctccc |
| | tegtgcgctctcctgttccgaccctgccgcttaccggatacctgtccgcctttctccct |
| | tcgggaagcgtggcgctttctcaatgctcacgctgtaggtatctcagttcggtgtaggt |
| | tatccggtaactatcgtcttgagtccaacccggtaagacacgacttatcgccactggca |
| | gcagccactggtaacaggattagcagagcgaggtatgtaggcggtgctacagagttctt |
| | gaagtggtggcctaactacggctacactagaaggacagtatttggtatctgcgctctgc |
| | tgaagccagttaccttcggaaaaagagttggtagctcttgatccggcaaacaaaccacc |
| | getggtageggtggtttttttgtttgcaagcagcagattacgcgcagaaaaaaaggatc |
| | tcaagaagatcctttgatcttttctacggggtctgacgctcagtggaacgaaaactcac |
| | gttaagggattttggtcatgagattatcaaaaaggatcttcacctagatccttttaaat |
| | taaaaatgaagttttaaatcaatctaaagtatatatgagtaaacttggtctgacagtta |
| | ttgcctgactccccgtcgtgtagataactacgatacgggagggcttaccatctggcccc |
| | agtgctgcaatgataccgcgagacccacgctcaccggctccagatttatcagcaataaa |
| | ccagccagccggaagggccgagcgcagaagtggtcctgcaactttatccgcctccatcc |
| | agtctattaattgttgccgggaagctagagtaagtagttcgccagttaatagtttgcgc |
| | aacgttgttgccattgctacaggcatcgtggtgtcacgctcgtcgtttggtatggcttc |
| | attcagctccggttcccaacgatcaaggcgagttacatgatcccccatgttgtgcaaaa |
| | aagcggttagctccttcggtcctccgatcgttgtcagaagtaagt |
| EGFR_Exon19del | ACTCTGGATCCCAGAAGGTGAGAAAGTTAAAATTCCCGTCGCTATCAAGGAATTAAGAG |
| (c.2236_2250del)_WT | AAGCAACATCTCCGAAAGGCAACAAGGAAATCCTCGATGTGAGTTTCTGCTTTGCT |
| (SEQ ID NO: 284) | |
| EGFR_Exon19del | ACTCTGGATCCCAGAAGGTGAGAAAGTTAAAATTCCCGTCGCTATCAAGACATCTCCGA |
| (c.2236_2250del)_MT | AAGGCAACAAGGAAATCCTCGATGTGAGTTTCTGCTTTGCT |
| (SEQ ID NO: 285) | |
| EGFR_p.T790M | GTGCCGCCTGCTGGGCATCTGCCTCACCTCCACCGTGCAGCTCATCACGCAGCTCATGC |
| (c.2269C > T)_WT | CCTTCGGCTGCCTCCTGGACTATGTCCGGGAACACAAAGAC |
| (SEQ ID NO: 286) | |
| EGFR_p.T790M | GTGCCGCCTGCTGGGCATCTGCCTCACCTCCACCGTGCAGCTCATCATGCAGCTCATGC |
| (c.2269C > T)_MT | CCTTCGGCTGCCTCCTGGACTATGTCCGGGAACACAAAGAC |
| (SEQ ID NO: 287) | |
| EGFR_p.L858R | CTTGGTGCACCGCGACCTGGCAGCCAGGAACGTACTGGTGAAAACACCGCAGCATGTCA |
| (c.2573T > G)_WT | AGATCACAGATTTTGGGCTGGCCAAACGGCTGGGTGCGGAAGAGAAAGA |
| (SEQ ID NO: 288) | |
| EGFR_p.L858R | CTTGGTGCACCGCGACCTGGCAGCCAGGAACGTACTGGTGAAAACACCGCAGCATGTCA |
| (c.2573T > G)_MT | AGATCACAGATTTTGGGCGGGCCAAACGGCTGGGTGCGGAAGAGAAAGA |
| (SEQ ID NO: 289) | |
| KRAS_p.G12D | ATGATTCTGAATTAGCTGTATCGTCAAGGCACTCTTGCCTACGCCACCAGCTCCAACTA |
| (c.35G > A)_WT | CCACAAGTTTATATTCAGTCATTTTCAGCAGGCCTTATAAT |
| (SEQ ID NO: 290) | |
| KRAS_p.G12D | ATGATTCTGAATTAGCTGTATCGTCAAGGCACTCTTGCCTACGCCATCAGCTCCAACTA |
| (c.35G > A)_MT | CCACAAGTTTATATTCAGTCATTTTCAGCAGGCCTTATAAT |
| (SEQ ID NO: 291) | |

### Digenome Sequencing

Digenome was carried out as described in Kim et al., Nature methods 12:237-243 (2015). Briefly, 8 ug genomic DNA (gDNA) was extracted from HEK293T using Blood Tissue kit (Qiagen), and then digested with 40 ug Cas9 and 10 ug gRNA (target sequence: 5′-TTGGACATACTGGATACAGC-3′; SEQ ID NO: 280) in 400 ul 1× Cutsmart buffer (New England Biolabs) at 37° C. for 16 hrs. Digested gDNA was isolated using Blood and Tissue kit (Qiagen) then fragmented at size of 500˜600 bp by M220 ultrasonicator (Covaris). NGS library for whole genome sequencing was prepared with a TruSeq Nano kit (Illumina), then sequenced by NovaSeq (illumine). The measure of the Double Strand Break score was preformed digenome analysis tool at Rgenome web server (rgenome.net) with 2 bp overhang in case of FnCas9. The loci with DSB score over 1 was sorted and marked as manhattan plot across human genome (hg38).

### Analysis of Cancer-Related Mutations Targetable by CRISPR Enrichment

Up-down 20 bp sequences of all PAM(NGG/CCN) sites were extracted from human genome (GRCh38). COSMIC point mutation data of cancers were downloaded (cancer.sanger.ac.uk/cosmic). The number of mutations located on all PAM(NGG/CCN) sites were counted and the number of mutations located inside of up-down 20 bp window sequences of all PAM(NGG/CCN) sites were counted. Ratio of the mutation count on/inside of PAM sites to total number of mutations were computed.

### Mutant DNA Enrichment by CRISPR

wtDNA and mutant of EGFR Exon19del, EGFR T790M, EGFR L858R and KRAS G12D are synthesized by IDT. A DNA samples was prepared by mixing the wtDNA and the mtDNA at a ratio of 95:5 (wtDNA ratio 95%; mtDNA ratio 5%), 99:1 (wtDNA ratio 99%; mtDNA ratio 1%), 99.9:0.1 (wtDNA ratio 99.9%; mtDNA ratio 0.1%), or 100:0 (wtDNA ratio 100%; mtDNA ratio 0%). 5 ng mutant/wtDNA mixture was digested by 500 ng FnCas9-AF2 with 200 ng guide in 10 ul 1× Cutsmart buffer (New England Biolabs) at 37° C. for 1 hr, which was terminated by adding 10× STOP R×N solution (1% SDS, 100 mM EDTA, pH 8). Digested products were then amplified by Q5 DNA polymerase (New England Biolabs) with Index primer. Index PCR amplicons were purified with AMPure and sequenced on an illumine Iseq instruments.

### Tissue/Blood Sampling and DNA/cfDNA Extraction

Patients with Non-small cell lung cancer who have Stage I were included in the study, which was approved by (IRB No. 2020AN0005). Cancer tissues were collected during surgical resection and about 10 cc of blood was collected in EDTA tubes (BD Vacutainer) prior to surgical resection. DNA was extracted from tissue with DNeasy Blood & Tissue Kits (Qiagen) according to the manufacturer's protocol. Blood was moved to falcon tube and centrifuged at 1900 g. Supernatants (plasma) were collected to e-tube and centrifuged again at 16000 g.

cfDNA was isolated from 1 ml of plasma with the Maxwell RSC ccfDNA plasma kit (Promega) following manufacture's instrument. cfDNA was eluted in 60 ul elution buffer in Maxwell RSC ccfDNA plasma kit. cfDNA was applied to the cell-fee DNA ScreenTape with Agilent 4150 TapeStation instruments (Agilent). Concentration and purity of cfDNA was analyzed using the Agilent TapeStation System software (Agilent).

Cancer Patient Mutant Allele Enrichment with CRISPR and NGS Library Preparation

Mutant allele enrichment NGS library were prepared from 5-10 ng of gDNA and DNA/cfDNA. 7 genes containing hotspot of interest were amplified by Q5 DNA polymerase (New England Biolabs) with 18 primer pairs (see Table 4).

A fraction (1 ul) of PCR product 10 fold diluted with DEPC water was treated by 8 ug (25 pmol/10 ul) FnCas9-AF2 with 2 ug (50 pmol/10 ul) gRNA Mix (see Table 5 which provides the sequences for the gRNAs used in the different experiments) in 10 ul 1×Cutsmart buffer (New England Biolabs) at 37° C. for 1 hr to remove wild type DNA allele, which was terminated by adding 10× STOP R×N solution (1% SDS, 100 mM EDTA, pH 8).

Wild type digested-products were then amplified by Q5 DNA polymerase (New England Biolabs) with Index primer. Index PCR amplicons were purified with AMPure and sequenced on an illumine Iseq instruments.

### Data Analysis

For accurate quantification of the mutation ratio changed according to the use of Cas9, cfDNA NGS data was analyzed using an in-house script (written in Python) instead of the popular NGS analysis method. Analysis was performed in three steps: QC, target read capture, and mutation detection.

To perform target read capture and mutation detection steps, target information including target amplicon site and mutation site was prepared. The target amplicon site extracted the sequence expected to be amplified through the gRNA sequence and primer information based on the reference genome sequence. Mutation site extracted the nucleotide sequence change of the mutation to be confirmed from the COSMIC database, and prepared the expected mutation site sequence.

In the initial quality control step, low quality read was trimmed using FASTQC tools (worldwideweb.bioinformatics.bbsrc.ac.uk/projects/fastqc) by phred quality score, remove adapter sequence, and perform length filter (min=50, max=150 bp).

The mutation rates for all variants were presented as the ratio of “reads including mutations/total reads” at the location of specific variants. For each ratio, it was organized in the form of R dataframe, and the heat map package was utilized and visualized. Boxplot was presented using the boxplot function of the pubr package of R, and statistical significance for each group was obtained through t-test and Kruskal-Wallis analysis for the entire group. The correlation plot was presented as a visualization strategy using the plot function and R cortest function.

TABLE 4
Primer Sequences

| Primer | Sequence |
|---|---|
| 3kb IVC template_F | ACTTACTTCTGACAACGATCGGAG |
| (SEQ ID NO: 10) | |
| 3kb IVC template_R | GGGAATAAGGGCGACACGGAA |
| (SEQ ID NO: 11) | |
| EGFR_NSCLC_T1_F | ACACTCTTTCCCTACACGACGCTCTTCCGATCTGAGAAAGTTAAAATTCCCGTCG |
| (SEQ ID NO: 12) | |
| EGFR_NSCLC_T1_R | GTGACTGGAGTTCAGACGTGTGCTCTTCCGATCTCACATCGAGGATTTCCTTGTT |
| (SEQ ID NO: 13) | GCCTTTCG |
| EGFR_NSCLC_T2_F | ACACTCTTTCCCTACACGACGCTCTTCCGATCTCCCTCCCTCCAGGAAGCCTACC |
| (SEQ ID NO: 14) | TGATG |
| EGFR_NSCLC_T2_R | GTGACTGGAGTTCAGACGTGTGCTCTTCCGATCTGGTGGAGGTGAGGCAGATG |
| (SEQ ID NO: 15) | |
| EGFR_NSCLC_T3_F | ACACTCTTTCCCTACACGACGCTCTTCCGATCTCTCACCTCCACCGTGCCGCTCA |
| (SEQ ID NO: 16) | |
| EGFR_NSCLC_T3_R | GTGACTGGAGTTCAGACGTGTGCTCTTCCGATCTCACACCAGTTGAGCAG |
| (SEQ ID NO: 17) | GTACTGGG |
| EGFR_NSCLC_T4_F | ACACTCTTTCCCTACACGACGCTCTTCCGATCTCTTGGTGCACCGCGACC |
| (SEQ ID NO: 18) | |
| EGFR_NSCLC_T4_R | GTGACTGGAGTTCAGACGTGTGCTCTTCCGATCTTTCTCTTCCGCACCCAGCCGT |
| (SEQ ID NO: 19) | TTG |
| KRAS_NSCLC_T1_F | ACACTCTTTCCCTACACGACGCTCTTCCGATCTCGTCAAGGCACTCTTGCC |
| (SEQ ID NO: 20) | |
| KRAS_NSCLC_T1_R | GTGACTGGAGTTCAGACGTGTGCTCTTCCGATCTTGCTGAAAATGACTGAATATA |
| (SEQ ID NO: 21) | AACTTGT |
| KRAS_NSCLC_T2_F | ACACTCTTTCCCTACACGACGCTCTTCCGATCTCTGTATTTATTTCAGTGTTACT |
| (SEQ ID NO: 22) | TACCTGTCTTGT |
| KRAS_NSCLC_T2_R | GTGACTGGAGTTCAGACGTGTGCTCTTCCGATCTGCTCAGGACTTAGCAAGAAGT |
| (SEQ ID NO: 23) | TATGGAATT |
| NRAS_NSCLC_T1_F | ACACTCTTTCCCTACACGACGCTCTTCCGATCTATTGTCAGTGCGCTTTTCC |
| (SEQ ID NO: 24) | |
| NRAS_NSCLC_T1_R | GTGACTGGAGTTCAGACGTGTGCTCTTCCGATCTTTGCTGGTGTGAAATGACTGA |
| (SEQ ID NO: 25) | |
| NRAS_NSCLC_T2_F | ACACTCTTTCCCTACACGACGCTCTTCCGATCTTGGTCTCTCATGGCACTGTCCT |
| (SEQ ID NO: 26) | CTT |
| NRAS_NSCLC_T2_R | GTGACTGGAGTTCAGACGTGTGCTCTTCCGATCTATGGTGAAACCTGTTTGTTGG |
| (SEQ ID NO: 27) | |
| TP53_NSCLC_T1_F | ACACTCTTTCCCTACACGACGCTCTTCCGATCGGTTTTCTGGGAAGGGACAGA |
| (SEQ ID NO: 28) | |
| TP53_NSCLC_T1_R | GTGACTGGAGTTCAGACGTGTGCTCTTCCGATCTCACCAGCAGCTCCTACACC |
| (SEQ ID NO: 29) | |
| TP53_NSCLC_T2_F | ACACTCTTTCCCTACACGACGCTCTTCCGATCTCACCATCGCTATCTGAGCACCG |
| (SEQ ID NO: 30) | CTC |
| TP53_NSCLC_T2_R | GTGACTGGAGTTCAGACGTGTGCTCTTCCGATCTTCTACAAGCAGTCACAGCACA |
| (SEQ ID NO: 31) | T |
| TP53_NSCLC_T3_F | ACACTCTTTCCCTACACGACGCTCTTCCGATCTGTGGCAAGTGGCTCCTGA |
| (SEQ ID NO: 32) | |
| TP53_NSCLC_T3_R | GTGACTGGAGTTCAGACGTGTGCTCTTCCGATCTTGTAACAGTTCCTGCATGGGC |
| (SEQ ID NO: 33) | CGCATGAAC |
| TP53_NSCLC_T4_F | ACACTCTTTCCCTACACGACGCTCTTCCGATCTACAAACACGCACCTCAAACCTG |
| (SEQ ID NO: 34) | TTCCG |
| TP53_NSCLC_T4_R | GTGACTGGAGTTCAGACGTGTGCTCTTCCGATCTTGCCTCTTGCTTCTCTTTTCC |
| (SEQ ID NO: 35) | T |
| TP53_NSCLC_T5_F | ACACTCTTTCCCTACACGACGCTCTTCCGATCTGTGAGGCTCCCCTTTCTTG |
| (SEQ ID NO: 36) | |
| TP53_NSCLC_T5_R | GTGACTGGAGTTCAGACGTGTGCTCTTCCGATCTCGTGTTTGTGCCTGTCCTG |
| (SEQ ID NO: 37) | |
| TP53_NSCLC_T6_F | ACACTCTTTCCCTACACGACGCTCTTCCGATCTCACCGCTTCTTGTCCTGCTTCC |
| (SEQ ID NO: 38) | TTACC |
| TP53_NSCLC_T6_R | GTGACTGGAGTTCAGACGTGTGCTCTTCCGATCTAAAGGGGAGCCTCACCAC |
| (SEQ ID NO: 39) | |
| PIK3CA_NSCLC_T1_F | ACACTCTTTCCCTACACGACGCTCTTCCGATCTTGAGCAAGAGGCTTTGGAGT |
| (SEQ ID NO: 40) | |
| PIK3CA_NSCLC_T1_R | GTGACTGGAGTTCAGACGTGTGCTCTTCCGATCTCCAATCCATTTTTGTTGTCCA |
| (SEQ ID NO: 41) | |
| PIK3CA_NSCLC_T2_F | ACACTCTTTCCCTACACGACGCTCTTCCGATCTTCCACACAATTAAACAGCCTGC |
| (SEQ ID NO: 42) | ATTGA |
| PIK3CA_NSCLC_T2_R | GTGACTGGAGTTCAGACGTGTGCTCTTCCGATCTTGGAATCCAGAGTGAGCTTTC |
| (SEQ ID NO: 43) | ATT |
| BRAF_NSCLC_T1_F | ACACTCTTTCCCTACACGACGCTCTTCCGATCTTCATGAAGACCTCACAGTAAAA |
| (SEQ ID NO: 44) | ATAGGT |
| BRAF_NSCLC_T1_R | GTGACTGGAGTTCAGACGTGTGCTCTTCCGATCTCTGATGGGACCCACTCCACCG |
| (SEQ ID NO: 45) | AG |
| BRAF_NSCLC_T2_F | ACACTCTTTCCCTACACGACGCTCTTCCGATCTGTGAAATCTCGATGGAGTGGGT |
| (SEQ ID NO: 46) | C |
| BRAF_NSCLC_T2_R | GTGACTGGAGTTCAGACGTGTGCTCTTCCGATCTTGGAAAAATAGCCTCAATTCT |
| (SEQ ID NO: 47) | TACCATCC |

TABLE 5
gRNA Target Sequences

| | SEQ ID | | |
|---|---|---|---|
| Experiment | NO | Name | Target sequence |
| Specificity | 48 | KRAS-WT | GUAGUUGGAGCUGGUGGCGU |
| comparison_ | 49 | KRAS-1 | GUAGUUGGAGCUGGUGGCGA |
| SpCas9 versus | 50 | KRAS-2 | GUAGUUGGAGCUGGUGGCCU |
| FnCas9 | 51 | KRAS-3 | GUAGUUGGAGCUGGUGGGGU |
| | 52 | KRAS-4 | GUAGUUGGAGCUGGUGCCGU |
| | 53 | KRAS-5 | GUAGUUGGAGCUGGUCGCGU |
| | 54 | KRAS-6 | GUAGUUGGAGCUGGAGGCGU |
| | 55 | KRAS-7 | GUAGUUGGAGCUGCUGGCGU |
| | 56 | KRAS-8 | GUAGUUGGAGCUCGUGGCGU |
| | 57 | KRAS-9 | GUAGUUGGAGCAGGUGGCGU |
| | 58 | KRAS-10 | GUAGUUGGAGGUGGUGGCGU |
| | 59 | KRAS-11 | GUAGUUGGACCUGGUGGCGU |
| | 60 | KRAS-12 | GUAGUUGGUGCUGGUGGCGU |
| | 51 | KRAS-13 | GUAGUUGCAGCUGGUGGCGU |
| | 62 | KRAS-14 | GUAGUUCGAGCUGGUGGCGU |
| | 63 | KRAS-15 | GUAGUAGGAGCUGGUGGCGU |
| | 64 | KRAS-16 | GUAGAUGGAGCUGGUGGCGU |
| | 65 | KRAS-17 | GUACUUGGAGCUGGUGGCGU |
| | 66 | KRAS-18 | GUUGUUGGAGCUGGUGGCGU |
| | 67 | KRAS-19 | GAAGUUGGAGCUGGUGGCGU |
| | 68 | KRAS-20 | CUAGUUGGAGCUGGUGGCGU |
| Specificity test | 105 | NRAS-WT | UUGGACAUACUGGAUACAGC |
| with mismatched | 106 | NRAS-1-G | UUGGACAUACUGGAUACAGG |
| gRNA to NRAS | 107 | NRAS-1-A | UUGGACAUACUGGAUACAGA |
| | 108 | NRAS-1-T | UUGGACAUACUGGAUACAGU |
| | 109 | NRAS-2-C | UUGGACAUACUGGAUACACC |
| | 110 | NRAS-2-A | UUGGACAUACUGGAUACAAC |
| | 111 | NRAS-2-T | UUGGACAUACUGGAUACAUC |
| | 112 | NRAS-3-T | UUGGACAUACUGGAUACUGC |
| | 113 | NRAS-3-G | UUGGACAUACUGGAUACGGC |
| | 114 | NRAS-3-C | UUGGACAUACUGGAUACCGC |
| | 115 | NRAS-4-G | UUGGACAUACUGGAUAGAGC |
| | 116 | NRAS-4-A | UUGGACAUACUGGAUAAAGC |
| | 117 | NRAS-4-T | UUGGACAUACUGGAUAUAGC |
| | 118 | NRAS-5-T | UUGGACAUACUGGAUUCAGC |
| | 119 | NRAS-5-G | UUGGACAUACUGGAUGCAGC |
| | 120 | NRAS-5-C | UUGGACAUACUGGAUCCAGC |
| | 121 | NRAS-6-A | UUGGACAUACUGGAAACAGC |
| | 122 | NRAS-6-G | UUGGACAUACUGGAGACAGC |
| | 123 | NRAS-6-C | UUGGACAUACUGGACACAGC |
| | 124 | NRAS-7-T | UUGGACAUACUGGUUACAGC |
| | 125 | NRAS-7-G | UUGGACAUACUGGGUACAGC |
| | 126 | NRAS-7-C | UUGGACAUACUGGCUACAGC |
| | 127 | NRAS-8-C | UUGGACAUACUGCAUACAGC |
| | 128 | NRAS-8-A | UUGGACAUACUGAAUACAGC |
| | 129 | NRAS-8-T | UUGGACAUACUGUAUACAGC |
| | 130 | NRAS-9-C | UUGGACAUACUCGAUACAGC |
| | 131 | NRAS-9-A | UUGGACAUACUAGAUACAGC |
| | 132 | NRAS-9-T | UUGGACAUACUUGAUACAGC |
| | 133 | NRAS-10-A | UUGGACAUACAGGAUACAGC |
| | 134 | NRAS-10-G | UUGGACAUACGGGAUACAGC |
| | 135 | NRAS-10-C | UUGGACAUACCGGAUACAGC |
| | 136 | NRAS-11-G | UUGGACAUAGUGGAUACAGC |
| | 137 | NRAS-11-A | UUGGACAUAAUGGAUACAGC |
| | 138 | NRAS-11-T | UUGGACAUAUUGGAUACAGC |
| | 139 | NRAS-12-T | UUGGACAUUCUGGAUACAGC |
| | 140 | NRAS-12-G | UUGGACAUGCUGGAUACAGC |
| | 141 | NRAS-12-C | UUGGACAUCCUGGAUACAGC |
| | 142 | NRAS-13-A | UUGGACAAACUGGAUACAGC |
| | 143 | NRAS-13-G | UUGGACAGACUGGAUACAGC |
| | 144 | NRAS-13-C | UUGGACACACUGGAUACAGC |
| | 145 | NRAS-14-T | UUGGACUUACUGGAUACAGC |
| | 146 | NRAS-14-G | UUGGACGUACUGGAUACAGC |
| | 147 | NRAS-14-C | UUGGACCUACUGGAUACAGC |
| | 148 | NRAS-15-G | UUGGAGAUACUGGAUACAGC |
| | 149 | NRAS-15-A | UUGGAAAUACUGGAUACAGC |
| | 150 | NRAS-15-T | UUGGAUAUACUGGAUACAGC |
| | 151 | NRAS-16-T | UUGGUCAUACUGGAUACAGC |
| | 152 | NRAS-16-G | UUGGGCAUACUGGAUACAGC |
| | 153 | NRAS-16-C | UUGGCCAUACUGGAUACAGC |
| | 154 | NRAS-17-C | UUGCACAUACUGGAUACAGC |
| | 155 | NRAS-17-A | UUGAACAUACUGGAUACAGC |
| | 156 | NRAS-17-T | UUGUACAUACUGGAUACAGC |
| | 157 | NRAS-18-C | UUCGACAUACUGGAUACAGC |
| | 158 | NRAS-18-A | UUAGACAUACUGGAUACAGC |
| | 159 | NRAS-18-T | UUUGACAUACUGGAUACAGC |
| | 160 | NRAS-19-A | UAGGACAUACUGGAUACAGC |
| | 161 | NRAS-19-G | UGGGACAUACUGGAUACAGC |
| | 162 | NRAS-19-C | UCGGACAUACUGGAUACAGC |
| | 163 | NRAS-20-A | AUGGACAUACUGGAUACAGC |
| | 164 | NRAS-20-G | GUGGACAUACUGGAUACAGC |
| | 165 | NRAS-20-C | CUGGACAUACUGGAUACAGC |
| Specifcity test | 48 | KRAS-WT | GUAGUUGGAGCUGGUGGCGU |
| with mismatched | 49 | KRAS-1-A (KRAS-1) | GUAGUUGGAGCUGGUGGCGA |
| gRNA to KRAS | 69 | KRAS-1-G | GUAGUUGGAGCUGGUGGCGG |
| | 70 | KRAS-1-C | GUAGUUGGAGCUGGUGGCGC |
| | 50 | KRAS-2-C (KRAS-2) | GUAGUUGGAGCUGGUGGCCU |
| | 71 | KRAS-2-A | GUAGUUGGAGCUGGUGGCAU |
| | 72 | KRAS-2-T | GUAGUUGGAGCUGGUGGCUU |
| | 51 | KRAS-3-G (KRAS-3) | GUAGUUGGAGCUGGUGGGGU |
| | 73 | KRAS-3-A | GUAGUUGGAGCUGGUGGAGU |
| | 74 | KRAS-3-T | GUAGUUGGAGCUGGUGGUGU |
| | 52 | KRAS-4-C (KRAS-4) | GUAGUUGGAGCUGGUGCCGU |
| | 75 | KRAS-4-A | GUAGUUGGAGCUGGUGACGU |
| | 76 | KRAS-4-T | GUAGUUGGAGCUGGUGUCGU |
| | 53 | KRAS-5-C (KRAS-5) | GUAGUUGGAGCUGGUCGCGU |
| | 77 | KRAS-5-A | GUAGUUGGAGCUGGUAGCGU |
| | 78 | KRAS-5-T | GUAGUUGGAGCUGGUUGCGU |
| | 54 | KRAS-6-A (KRAS-6) | GUAGUUGGAGCUGGAGGCGU |
| | 79 | KRAS-6-G | GUAGUUGGAGCUGGGGGCGU |
| | 80 | KRAS-6-C | GUAGUUGGAGCUGGCGGCGU |
| | 55 | KRAS-7-C (KRAS-7) | GUAGUUGGAGCUGCUGGCGU |
| | 81 | KRAS-7-A | GUAGUUGGAGCUGAUGGCGU |
| | 82 | KRAS-7-T | GUAGUUGGAGCUGUUGGCGU |
| | 56 | KRAS-8-C (KRAS-8) | GUAGUUGGAGCUCGUGGCGU |
| | 83 | KRAS-8-A | GUAGUUGGAGCUAGUGGCGU |
| | 84 | KRAS-8-T | GUAGUUGGAGCUUGUGGCGU |
| | 57 | KRAS-9-A (KRAS-9) | GUAGUUGGAGCAGGUGGCGU |
| | 85 | KRAS-9-G | GUAGUUGGAGCGGGUGGCGU |
| | 86 | KRAS-9-C | GUAGUUGGAGCCGGUGGCGU |
| | 58 | KRAS-10-G (KRAS-10) | GUAGUUGGAGGUGGUGGCGU |
| | 87 | KRAS-10-A | GUAGUUGGAGAUGGUGGCGU |
| | 88 | KRAS-10-T | GUAGUUGGAGUUGGUGGCGU |
| | 59 | KRAS-11-C (KRAS-11) | GUAGUUGGACCUGGUGGCGU |
| | 89 | KRAS-11-A | GUAGUUGGAACUGGUGGCGU |
| | 90 | KRAS-11-T | GUAGUUGGAUCUGGUGGCGU |
| | 60 | KRAS-12-T (KRAS-12) | GUAGUUGGUGCUGGUGGCGU |
| | 91 | KRAS-12-G | GUAGUUGGGGCUGGUGGCGU |
| | 92 | KRAS-12-C | GUAGUUGGCGCUGGUGGCGU |
| | 61 | KRAS-13-C (KRAS-13) | GUAGUUGCAGCUGGUGGCGU |
| | 93 | KRAS-13-A | GUAGUUGAAGCUGGUGGCGU |
| | 94 | KRAS-13-T | GUAGUUGUAGCUGGUGGCGU |
| | 62 | KRAS-14-C (KRAS-14) | GUAGUUCGAGCUGGUGGCGU |
| | 95 | KRAS-14-A | GUAGUUAGAGCUGGUGGCGU |
| | 96 | KRAS-14-T | GUAGUUUGAGCUGGUGGCGU |
| | 63 | KRAS-15-A (KRAS-15) | GUAGUAGGAGCUGGUGGCGU |
| | 97 | KRAS-15-G | GUAGUGGGAGCUGGUGGCGU |
| | 98 | KRAS-15-C | GUAGUCGGAGCUGGUGGCGU |
| | 64 | KRAS-16-A (KRAS-16) | GUAGAUGGAGCUGGUGGCGU |
| | 99 | KRAS-16-G | GUAGGUGGAGCUGGUGGCGU |
| | 100 | KRAS-16-C | GUAGCUGGAGCUGGUGGCGU |
| | 65 | KRAS-17-C (KRAS-17) | GUACUUGGAGCUGGUGGCGU |
| | 101 | KRAS-17-A | GUAAUUGGAGCUGGUGGCGU |
| | 102 | KRAS-17-T | GUAUUUGGAGCUGGUGGCGU |
| | 66 | KRAS-18-T (KRAS-18) | GUUGUUGGAGCUGGUGGCGU |
| | 103 | KRAS-18-G | GUGGUUGGAGCUGGUGGCGU |
| | 104 | KRAS-18-C | GUCGUUGGAGCUGGUGGCGU |
| | 67 | KRAS-19-A (KRAS-19) | GAAGUUGGAGCUGGUGGCGU |
| | 293 | KRAS-19-G | GGAGUUGGAGCUGGUGGCGU |
| | 294 | KRAS-19-C | GCAGUUGGAGCUGGUGGCGU |
| | 68 | KRAS-20-C (KRAS-20) | CUAGUUGGAGCUGGUGGCGU |
| | 295 | KRAS-20-A | AUAGUUGGAGCUGGUGGCGU |
| | 296 | KRAS-20-T | UUAGUUGGAGCUGGUGGCGU |
| Specifcity test | 166 | EGFR-WT | CAGAUUUUGGGCUGGCCAAA |
| with mismatched | 167 | EGFR-1-T | CAGAUUUUGGGCUGGCCAAU |
| gRNA to EGFR | 168 | EGFR-1-G | CAGAUUUUGGGCUGGCCAAG |
| | 169 | EGFR-1-C | CAGAUUUUGGGCUGGCCAAC |
| | 170 | EGFR-2-T | CAGAUUUUGGGCUGGCCAUA |
| | 171 | EGFR-2-G | CAGAUUUUGGGCUGGCCAGA |
| | 172 | EGFR-2-C | CAGAUUUUGGGCUGGCCACA |
| | 173 | EGFR-3-T | CAGAUUUUGGGCUGGCCUAA |
| | 174 | EGFR-3-G | CAGAUUUUGGGCUGGCCGAA |
| | 175 | EGFR-3-C | CAGAUUUUGGGCUGGCCCAA |
| | 176 | EGFR-4-G | CAGAUUUUGGGCUGGCGAAA |
| | 177 | EGFR-4-A | CAGAUUUUGGGCUGGCAAAA |
| | 178 | EGFR-4-T | CAGAUUUUGGGCUGGCUAAA |
| | 179 | EGFR-5-G | CAGAUUUUGGGCUGGGCAAA |
| | 180 | EGFR-5-A | CAGAUUUUGGGCUGGACAAA |
| | 181 | EGFR-5-T | CAGAUUUUGGGCUGGUCAAA |
| | 182 | EGFR-6-C | CAGAUUUUGGGCUGCCCAAA |
| | 183 | EGFR-6-A | CAGAUUUUGGGCUGACCAAA |
| | 184 | EGFR-6-T | CAGAUUUUGGGCUGUCCAAA |
| | 185 | EGFR-7-C | CAGAUUUUGGGCUCGCCAAA |
| | 186 | EGFR-7-A | CAGAUUUUGGGCUAGCCAAA |
| | 187 | EGFR-7-T | CAGAUUUUGGGCUUGCCAAA |
| | 188 | EGFR-8-A | CAGAUUUUGGGCAGGCCAAA |
| | 189 | EGFR-8-G | CAGAUUUUGGGCGGGCCAAA |
| | 190 | EGFR-8-C | CAGAUUUUGGGCCGGCCAAA |
| | 191 | EGFR-9-G | CAGAUUUUGGGGUGGCCAAA |
| | 192 | EGFR-9-A | CAGAUUUUGGGAUGGCCAAA |
| | 193 | EGFR-9-T | CAGAUUUUGGGUUGGCCAAA |
| | 194 | EGFR-10-C | CAGAUUUUGGCCUGGCCAAA |
| | 195 | EGFR-10-A | CAGAUUUUGGACUGGCCAAA |
| | 196 | EGFR-10-T | CAGAUUUUGGUCUGGCCAAA |
| | 197 | EGFR-11-C | CAGAUUUUGCGCUGGCCAAA |
| | 198 | EGFR-11-A | CAGAUUUUGAGCUGGCCAAA |
| | 199 | EGFR-11-T | CAGAUUUUGUGCUGGCCAAA |
| | 200 | EGFR-12-C | CAGAUUUUCGGCUGGCCAAA |
| | 201 | EGFR-12-A | CAGAUUUUAGGCUGGCCAAA |
| | 202 | EGFR-12-T | CAGAUUUUUGGCUGGCCAAA |
| | 203 | EGFR-13-A | CAGAUUUAGGGCUGGCCAAA |
| | 204 | EGFR-13-G | CAGAUUUGGGGCUGGCCAAA |
| | 205 | EGFR-13-C | CAGAUUUCGGGCUGGCCAAA |
| | 206 | EGFR-14-A | CAGAUUAUGGGCUGGCCAAA |
| | 207 | EGFR-14-G | CAGAUUGUGGGCUGGCCAAA |
| | 208 | EGFR-14-C | CAGAUUCUGGGCUGGCCAAA |
| | 209 | EGFR-15-A | CAGAUAUUGGGCUGGCCAAA |
| | 210 | EGFR-15-G | CAGAUGUUGGGCUGGCCAAA |
| | 211 | EGFR-15-C | CAGAUCUUGGGCUGGCCAAA |
| | 212 | EGFR-16-A | CAGAAUUUGGGCUGGCCAAA |
| | 213 | EGFR-16-G | CAGAGUUUGGGCUGGCCAAA |
| | 214 | EGFR-16-C | CAGACUUUGGGCUGGCCAAA |
| | 215 | EGFR-17-T | CAGUUUUUGGGCUGGCCAAA |
| | 216 | EGFR-17-G | CAGGUUUUGGGCUGGCCAAA |
| | 217 | EGFR-17-C | CAGCUUUUGGGCUGGCCAAA |
| | 218 | EGFR-18-C | CACAUUUUGGGCUGGCCAAA |
| | 219 | EGFR-18-A | CAAAUUUUGGGCUGGCCAAA |
| | 220 | EGFR-18-T | CAUAUUUUGGGCUGGCCAAA |
| | 221 | EGFR-19-T | CUGAUUUUGGGCUGGCCAAA |
| | 222 | EGFR-19-G | CGGAUUUUGGGCUGGCCAAA |
| | 223 | EGFR-19-C | CCGAUUUUGGGCUGGCCAAA |
| | 224 | EGFR-20-G | GAGAUUUUGGGCUGGCCAAA |
| | 225 | EGFR-20-A | AAGAUUUUGGGCUGGCCAAA |
| | 226 | EGFR-20-T | UAGAUUUUGGGCUGGCCAAA |
| Digenome-seq | 105 | SpCas9-NRAS-WT | UUGGACAUACUGGAUACAGC |
| | 105 | FnCas9-NRAS-WT | UUGGACAUACUGGAUACAGC |
| NSCLC DNA | 227 | FnCas9-EGFR-NSCLC_T1 | AGAGAAGCAACAUCUCCGAA |
| analysis | 228 | FnCas9-EGFR-NSCLC_T2 | GGUUGUCCACGCUGGCCAUC |
| | 229 | FnCas9-EGFR-NSCLC_T3 | GGCAUGAGCUGCGUGAUGAG |
| | 230 | FnCas9-EGFR-NSCLC_T4 | CAGAUUUUGGGCUGGCCAAA |
| | 48 | KRAS-NSCLC_T1 | GUAGUUGGAGCUGGUGGCGU |
| | | (KRAS-WT) | |
| | 231 | FnCas9-KRAS-NSCLC_T2 | AUUGAAACAUCAGCAAAGAC |
| | 232 | FnCas9-NRAS-NSCLC_T1 | UGGUUGGAGCAGGUGGUGUU |
| | 233 | FnCas9-NRAS-NSCLC_T2 | GGAUACAGCUGGACAAGAAG |
| | 234 | FnCas9-TP53-NSCLC_T1 | GCCAGGAGGGGGCUGGUGCA |
| | 235 | FnCas9-TP53-NSCLC_T2 | GGCGCUGCCCCCACCAUGAG |
| | 236 | FnCas9-TP53-NSCLC_T3 | GGAUGGGCCUCCGGUUCAUG |
| | 237 | FnCas9-TP53-NSCLC_T4 | GUAAUCUACUGGGACGGAAC |
| | 238 | FnCas9-TP53-NSCLC_T5 | UCUGUGCGCCGGUCUCUCCC |
| | 239 | FnCas9-TP53-NSCLC_T6 | AGGGAGCACUAAGCGAGGUA |
| | 240 | FnCas9-PIK3CA-NSCLC_T1 | AUGAUGCACAUCAUGGUGGC |
| | 241 | FnCas9-PIK3CA-NSCLC_T2 | UUAUCUUUUCAGUUCAAUGC |
| | 242 | FnCas9-BRAF-NSCLC_T1 | GUCUAGCUACAGUGAAAUCU |
| | 243 | FnCas9-BRAF-NSCLC_T2 | GUCUGGAUCCAUUUUGUGGA |

### Example 2: Analysis of the Effect of Base Mismatch on Cas9 Endonuclease Activity

To assess the effect that a base mismatch between the sgRNA and a target DNA sequence has on Cas9 activity, sgRNAs (20 total) with single-base mismatches at the different positions of a KRAS target sequence (20-base pairs long) were prepared as described in Example 1. Table 5 (above) provides the sequences for the KRAS-targeting sgRNAs. The specific single-base mismatch of the sgRNAs is shown bolded an in lower case. Then, the activity of the following Cas9 proteins was assessed with the different KRAS-targeting sgRNAs using an in vitro cleavage assay: (1) wild-type SpCas9, (ii) SpCas9-HF1, (iii) SpCas9-HF4, (iv) eSpCas9(1.0), (v) eSpCas9(1.1), and (vi) FnCas9-WT.

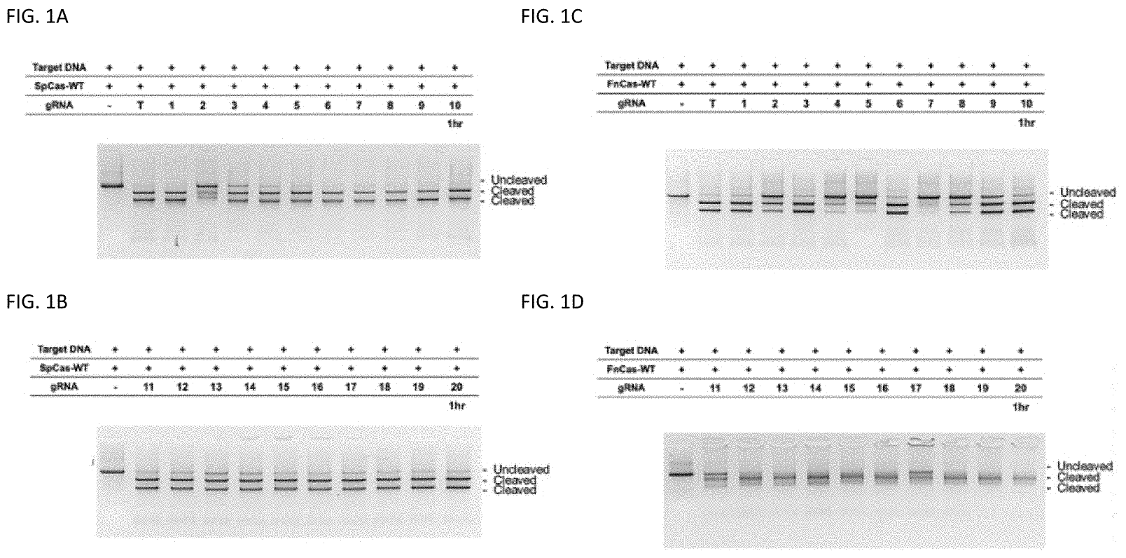

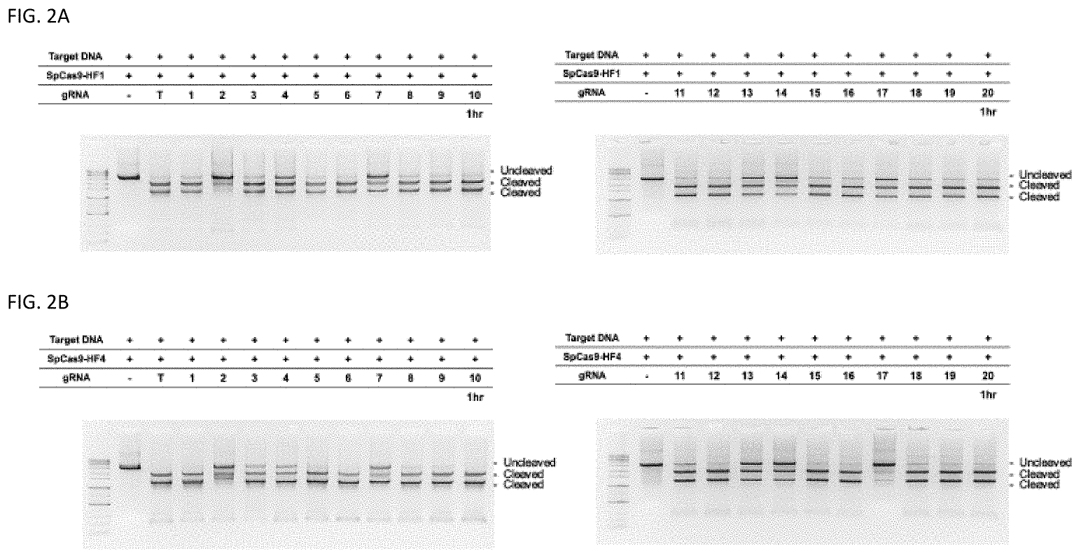

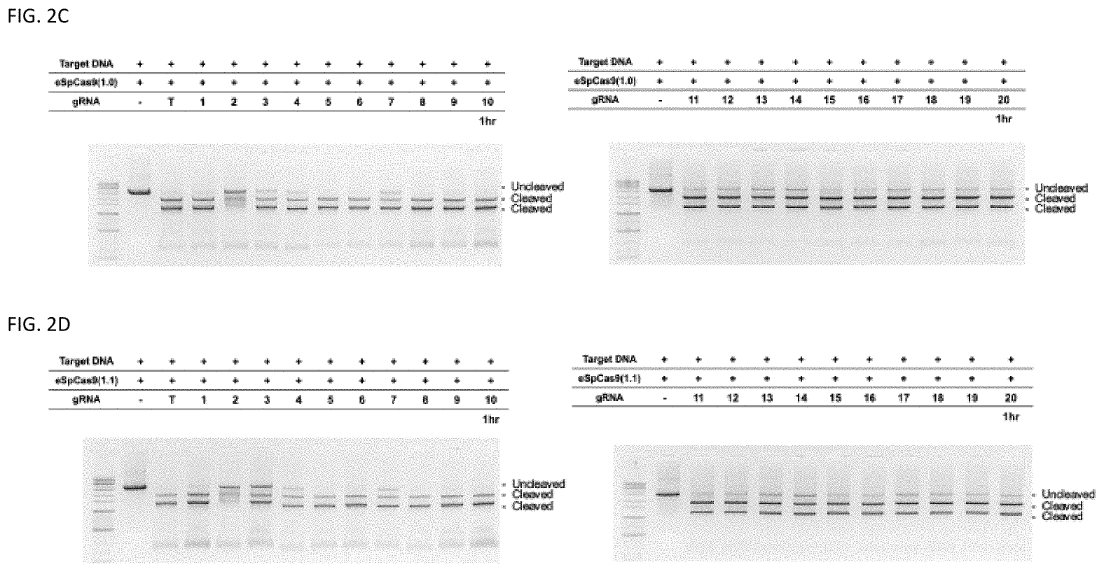

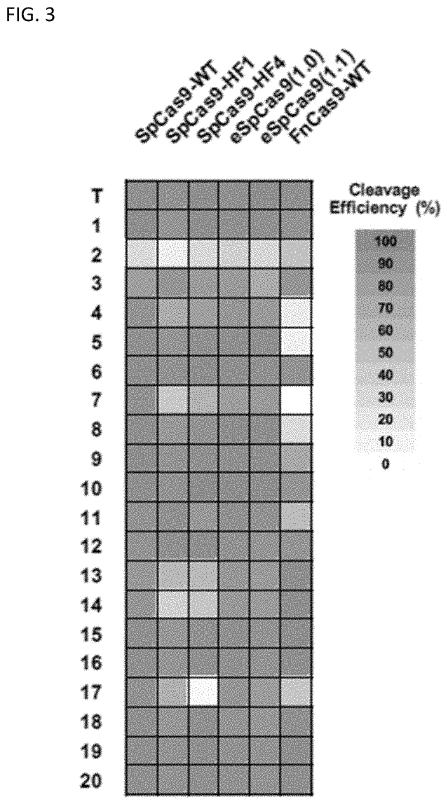

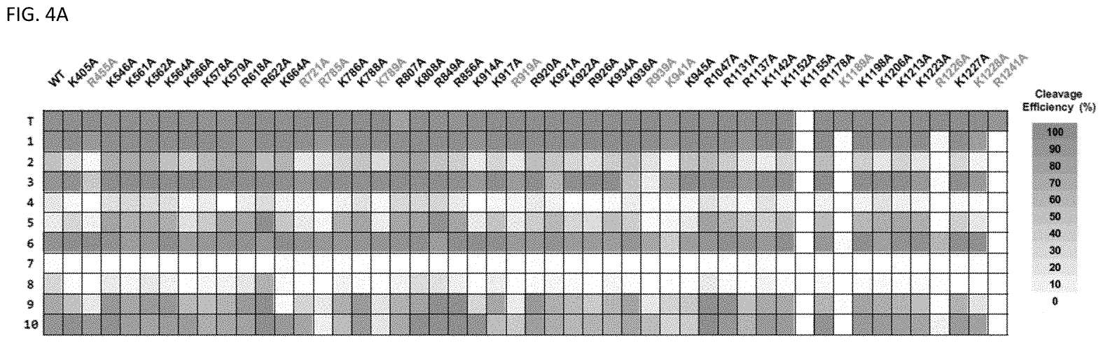

As shown in FIGS. 1A and 1B, except for with the KRAS-sgRNA #2, the wild-type SpCas9 induced significant cleavage of the KRAS target sequence with all other sgRNAs tested. The extent of cleavage was similar to that observed with the control KRAS-sgRNA, which did not contain any base mismatches. With SpCas9-HF1 and SpCas9-HF4, noticeable decreases in target DNA cleavage were observed only with KRAS-sgRNAs #2, 7, 13, 14, and 17 (see FIGS. 2A and 2B). As between SpCas9-HF1 and SpCas9-HF4, the decrease in cleavage efficiency was more prominent. With both eSpCas9(1.0) and eSpCas9(1.1), specificity was similar to that observed with the wild-type SpCas9 protein. Except with KRAS-sgRNA #2, significant cleavage of the KRAS target sequence was observed (see FIGS. 2C and 2D). Lastly, as with the wild-type FnCas9 protein, significantly reduced cleavage was observed with many more sgRNAs—i.e., KRAS-sgRNAs #2, 4, 7, 8, 9, 11, and 17 (see FIGS. 1C and 1D). A heat map comparison of the cleavage efficiency of the above Cas9 proteins with the different KRAS-sgRNAs is provided in FIG. 3.

The above results demonstrate that at least compared to SpCas9 and its high-specificity variants, the wild-type FnCas9 protein is much more efficient at discriminating single-base differences in a target sequence.

### Example 3: Construction of FnCas9 Protein Variants with Enhanced Specificity

To assess whether the specificity of the wild-type FnCas9 protein can be further enhanced, 49 different recombinant FnCas9 proteins with single amino acid modification were constructed. The amino acid modification was made at a residue within the cavity domain of the wild-type FnCas9 protein (SEQ ID NO: 1) that was predicted to interact with the backbone phosphate of a target DNA sequence. The FnCas9 protein variants contained an alanine substitution at one of the following amino acid residues: K405, R455, K546, K561, K562, K564, K566, K578, K579, R618, R622, K664, R721, R785, K786, K788, K789, R807, K808, R849, R856, K914, K917, R919, R920, K921, K922, R926, K934, K936, R939, K941, K945, R1047, R1131, R1137, K1142, K1152, K1155, R1178, K1189, K1198, K1206, K1213, K1223, R1226, K1227, K1228, and R1241. The cleavage efficiency of the different FnCas9 protein variants was tested with the KRAS-sgRNAs described in Example 2 using an in vitro cleavage assay.

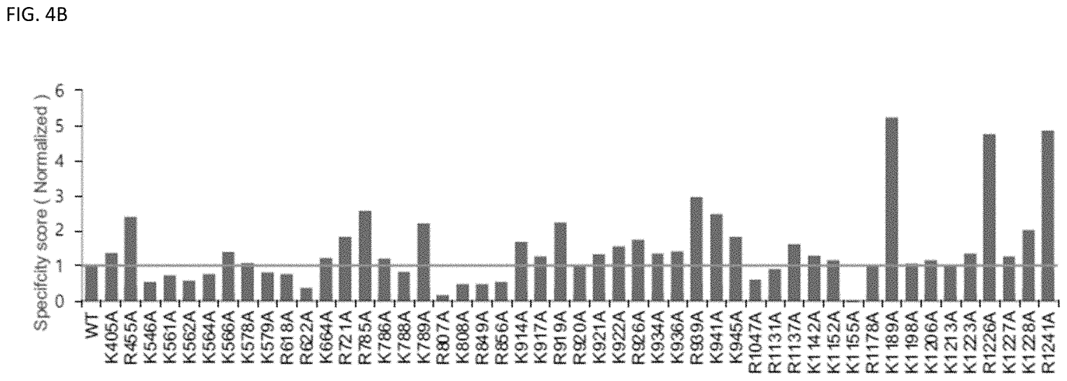

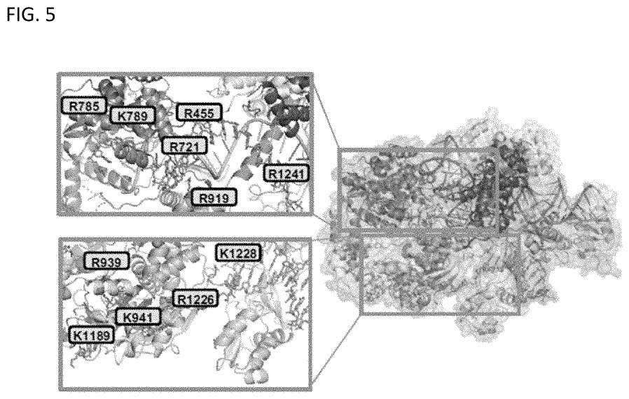

As shown in FIG. 4A, several of the amino acid modifications (e.g., R455A, R721A, R785A, K789A, R919A, R939A, K941A, K1189A, R1226A, K1228A, and R1241A) significantly reduced cleavage rate with one or more of the mismatched sgRNAs, while retaining cleavage capability with perfectly matched sgRNA. To compare the relative specificity of the FnCas9 single-residue variants, a specificity score (i.e., the differences between the cleavage rate of the non-target and average of off-targets) was determined for each of the variants. Of those tested, FnCas9 variants with an alanine substitution at one of the following amino acid residues had a specificity score above 60%: R455, R785, R721, K789, R919, R1241, R939, K941, K1189, R1226, and K1228 (see FIG. 4B). Amino acid residues R455, R785, R721, K789, R919, and R1241 were within the recognition (REC) lobe and determined to interact with the backbone phosphates of the target DNA strand (see FIG. 5). Amino acid residues R939, K941, K1189, R1226, and K1228 were within the nuclease (NUC) lobe and determined to interact with the backbone phosphates of non-target DNA strain. Among these 11 variants, FnCas9 protein with modifications at residue K1189 or R1241 had the highest specificity score.

Next, to assess whether specificity can be further enhanced, FnCas9 proteins with double and triple amino acid modifications were constructed. Specifically, FnCas9 protein variants with the following amino acid modifications were constructed: (i) K1189A and R1241A (“FnCas9 double mutant #1”) (also referred to herein as “FnCas9-advanced fidelity 1” or “FnCas9-AF1”); (ii) R721A and R1241A (“FnCas9 double mutant #2”); (iii) R785A and R1241A (“FnCas9 double mutant #3”); (iv) K1189A and R1241A (“FnCas9 double mutant #3); (v) R785A, K1189A, R1241A (“FnCas9 triple mutant #1”) (also referred to herein as “FnCas9-advanced fidelity 2” or “FnCas9-AF2”); (vi) R721A, K1189A, and R1241A (“FnCas9 triple mutant #2”); and (vii) K1189, K1228, and R1241 (“FnCas9 triple mutant #3”). Then, the in vitro cleavage rates of the FnCas9 double and triple mutants were assessed using sgRNAs (60 total) that cover all possible single-base mismatches in all 20 positions within the target NRAS sequence. The NRAS-targeting sgRNAs were produced as described in Example 1. Table 5 provides the sgRNA sequences.

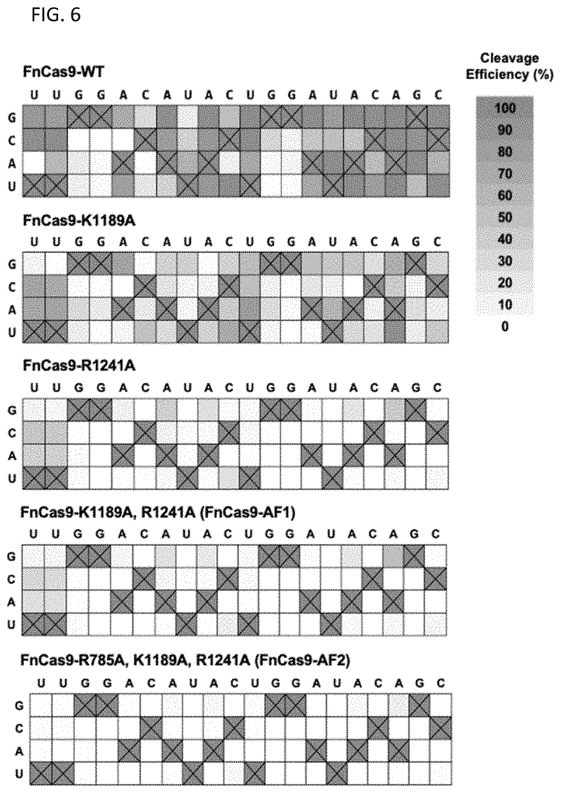

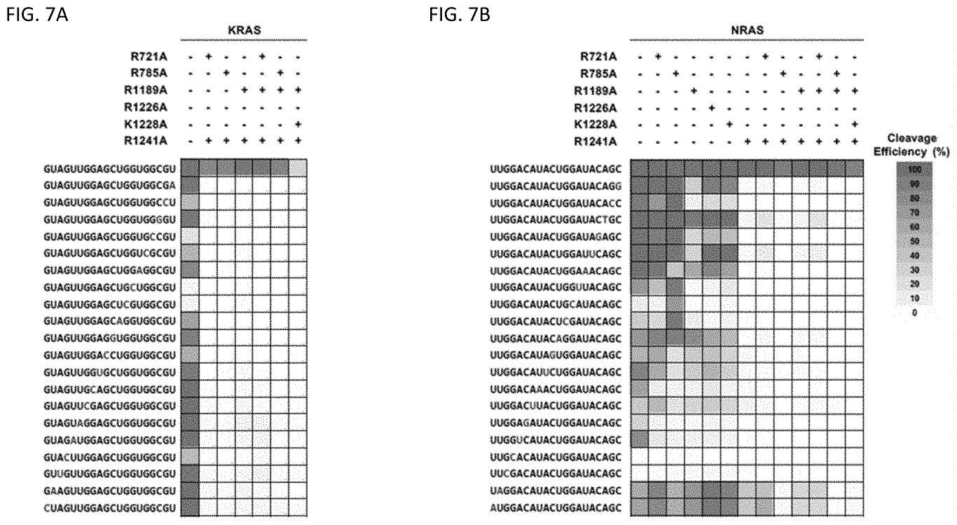

As shown in FIG. 6, compared to the wild-type and single mutant (K1189A or R1241A) FnCas9 proteins, the FnCas9 double mutant #1 (both K1189A and R1241A) showed greater specificity, as evidenced by the reduced cleavage of the NRAS target sequence with many of the NRAS-sgRNAs comprising a single-base mismatch. Similar results were observed with FnCas9 double mutants #2 and #3 (see FIG. 7B). With the triple mutants, even greater specificity was observed (see FIGS. 6 and 7B). As further confirmation, the cleavage efficiency of the double and triple FnCas9 protein variants was also tested with the KRAS-sgRNAs comprising a single-base mismatch described in Example 2. As shown in FIG. 7A, similar results were observed, with both the double and triple mutants exhibiting enhanced specificity, with the greatest specificity observed with the triple mutants.

The above results collectively demonstrate the enhanced specificity of the FnCas9 proteins described herein and suggest that certain amino acid modifications, particularly at residues that interact with the backbone phosphate of DNA sequences, could be useful in improving the specificity of a Cas9 protein.

### Example 4: Analysis of the Effect of SgRNA Sequence on the Specificity of FnCas9 Protein

To assess whether a particular sequence of a sgRNA described herein has an effect on the enhanced specificity of the above-described FnCas9 proteins, an in vitro cleavage assay was performed using FnCas9-AF2 (i.e., FnCas9 triple mutant #1) and sgRNAs comprising a single-base mismatch against the different positions of the target KRAS or EGFR target sequences (20-base pairs long). The sgRNAs were constructed as described in Example 1, and Table 5 provides the sequences of the sgRNAs.

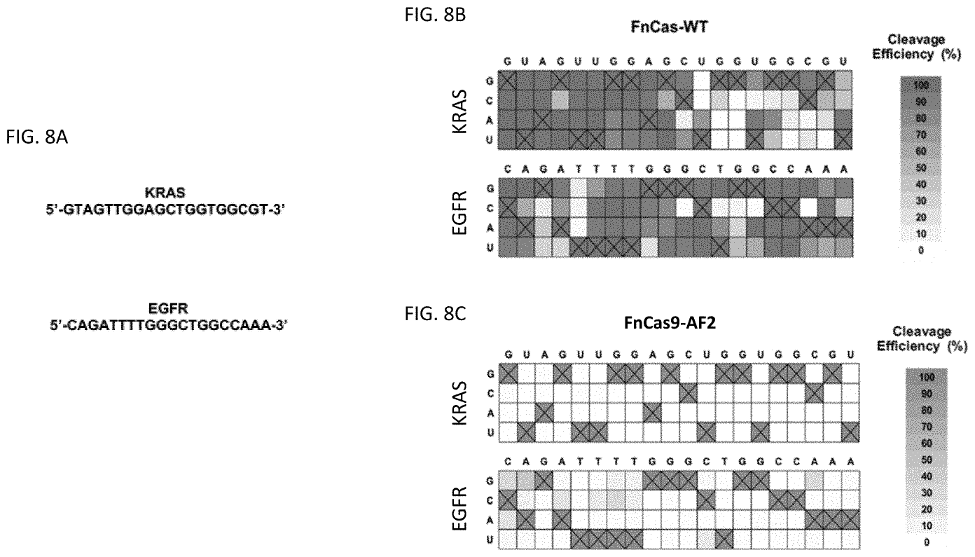

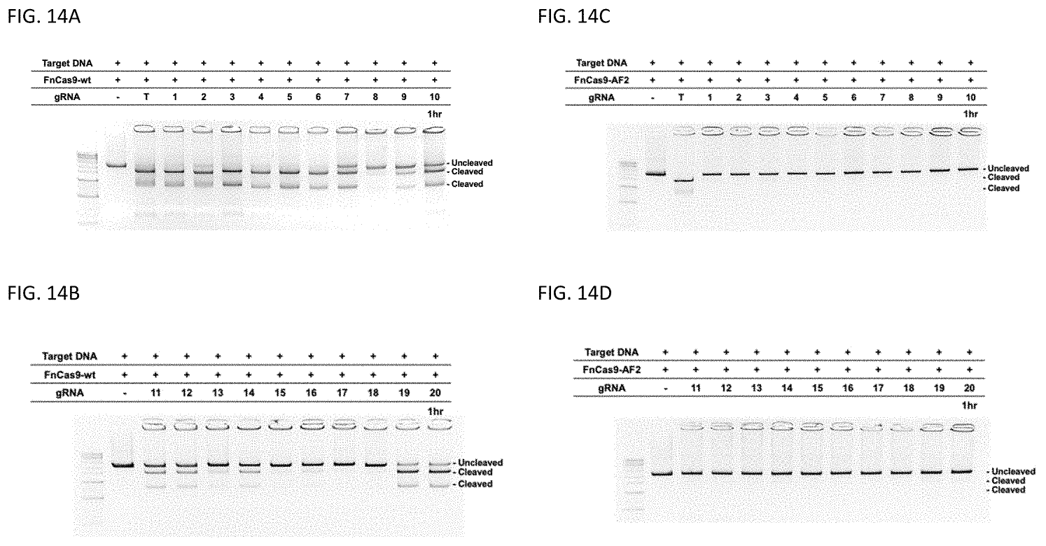

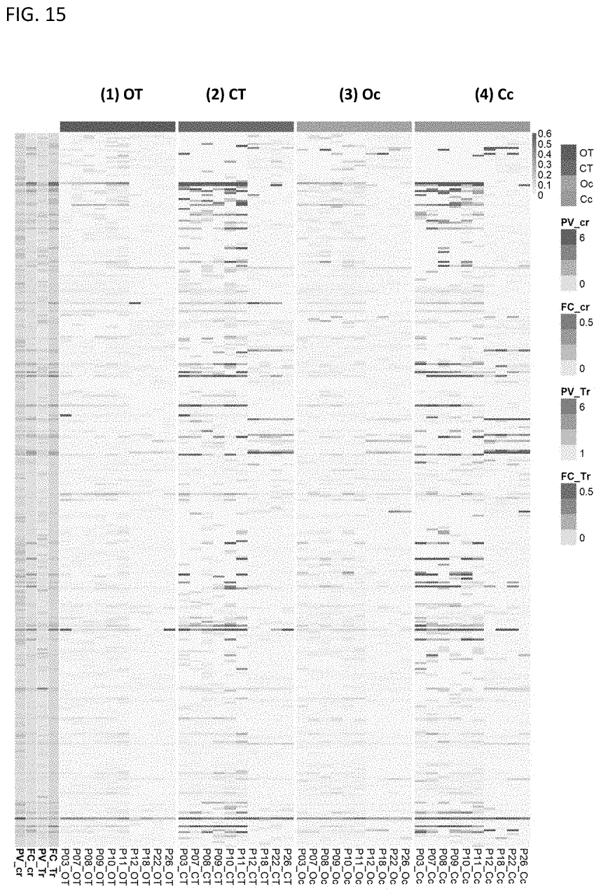

As shown in FIGS. 8B, 8C, and 14A-14D, the specificity of the FnCas9-AF2 with the KRAS and EGFR sgRNAs were comparable to that observed earlier with the NRAS-targeting sgRNAs (see Example 3). The results shown here demonstrate that the enhanced specificity of the FnCas9-AF2 is applicable to sgRNAs with various sequences.

### Example 5: Off-Target Analysis of FnCas9 Proteins with Enhanced Specificity

To assess whether the enhanced specificity of the FnCas9 proteins described herein is associated with decreased off-target effects, a genome-wide off-target analysis using Digenome-seq was conducted (as described in Example 1) for the following Cas9 proteins: (i) wild-type SpCas9 protein; (ii) wild-type FnCas9 protein; (iii) eSpCas9(1.1); (iv) FnCas9-AF1; (v) SpCas9-H4; and (vi) FnCas9-AF2.

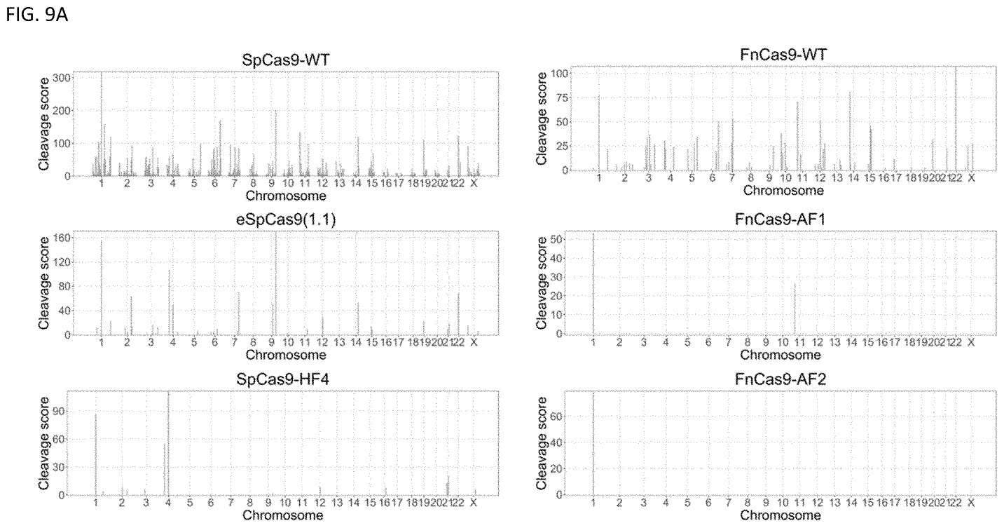

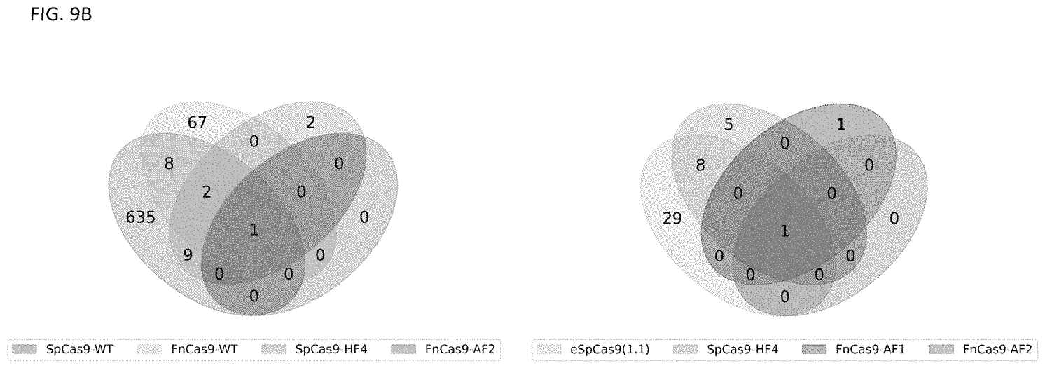

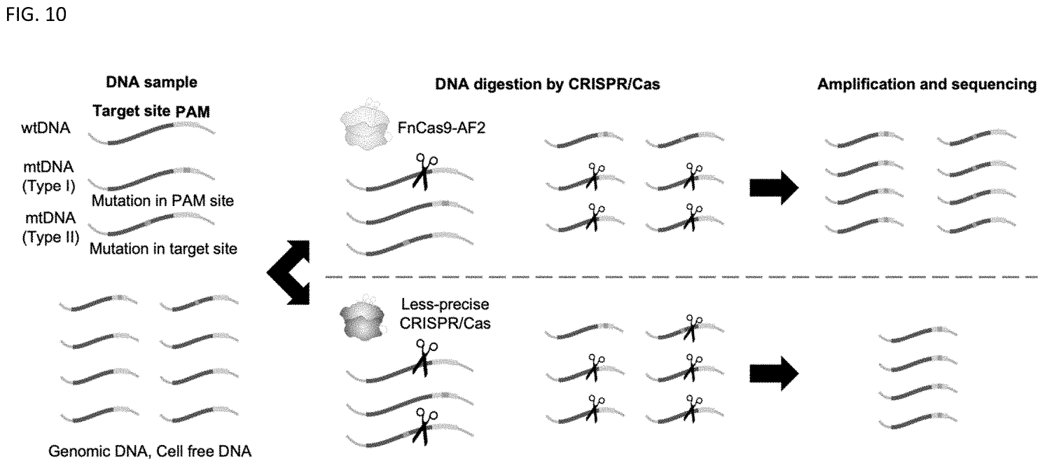

As shown in FIGS. 9A and 9B, with the wild-type SpCas9 protein there were 654 potential off-target sites with Digenome-seq cleavage score above 1. And, in agreement with the enhanced specificity observed in the earlier examples (see, e.g., Example 1), significantly reduced number of potential off-target sites were observed for the other Cas9 proteins tested. For example, with the wild-type FnCas9 protein, there were 77 potential off-target sites observed. With eSpCas9(1.1) and SpCas9-H4, there were 37 and 13 potential off-target sites, respectively. And, as to the FnCas9-AF1 and FnCas9-AF2 variants described herein, there were 1 and 0 potential off-target sites observed, respectively.

The above results confirm the enhanced specificity of the FnCas9 protein variants described herein, and suggest that with the FnCas9 proteins described herein are less prone to off-target effects observed with many of the Cas9 proteins available in the art (e.g., wild-type SpCas9 protein).

### Example 6: Enrichment of Low-Frequency Gene Mutations Using FnCas9 Proteins with Enhanced Selectivity

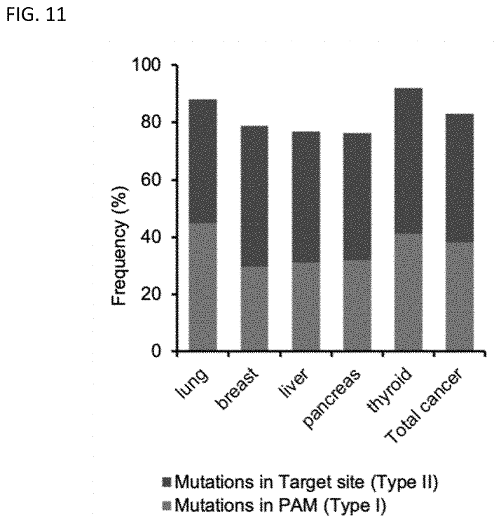

As described herein, current CRISPR-based enrichment methods (e.g., used to identify the presence of a circulating tumor DNA in a biological sample) requires the mutations to be positioned within the PAM region of a target gene sequence. Lee et al., Oncogene 36: 6823-6829 (2017). Accordingly, such methods are not capable of identifying mutations elsewhere within the target gene sequence. And, analysis of cancer-related mutations from COSMIC database showed that the current CRISPR-based enrichment methods would only be applicable for about 30% of all mutations (see FIG. 11). With the FnCas9 protein variants described herein (e.g., having enhanced specificity and capable of distinguishing a single nucleotide difference both within the PAM and outside the PAM region), in some aspects, DNA sequences comprising nearly 95% of cancer-related mutations could be identified.

To further assess the specificity of the FnCas9 proteins for pathogenic mutations (e.g., those associated with a cancer), an in vitro enrichment experiment was performed to screen for synthetic DNA comprising one of the following mutations, which were all positioned outside of the PAM region: EGFR Exon19del, EGFR T790M, EGFR L858R, and KRAS G12D. Mixtures comprising the following ratio of mutant to wild-type DNA sequences were prepared: 5%, 1%, 0.1%, or 0%. Then, the mixtures were digested with the FnCas9-AF2 protein (i.e., FnCas9 triple mutant #1) to see if the FnCas9 protein variant can be used to enrich for the mutant allele within the different mixtures. Next generation sequencing was used to determine the frequency of the mutant alleles within the different mixture both before and after the digestion.

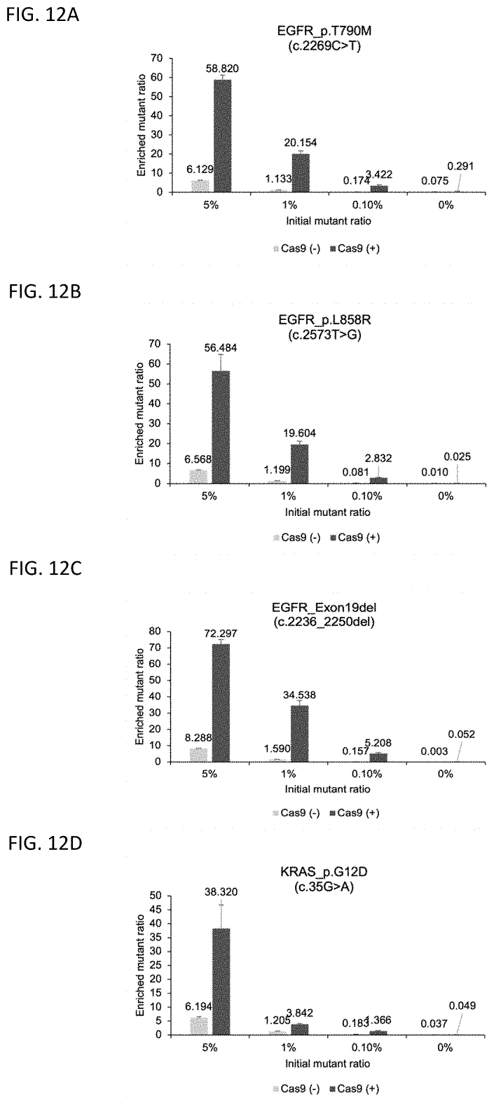

Prior to the digestion with the FnCas9 protein variant, the frequency of the mutant allele within the different mixtures was about as expected (FIGS. 12A-12C). However, after the digestion, the frequency of the mutant allele was significantly increased. Notably, no enrichment was observed in the negative control mixture (i.e., containing 0% of mutant DNA sequence).

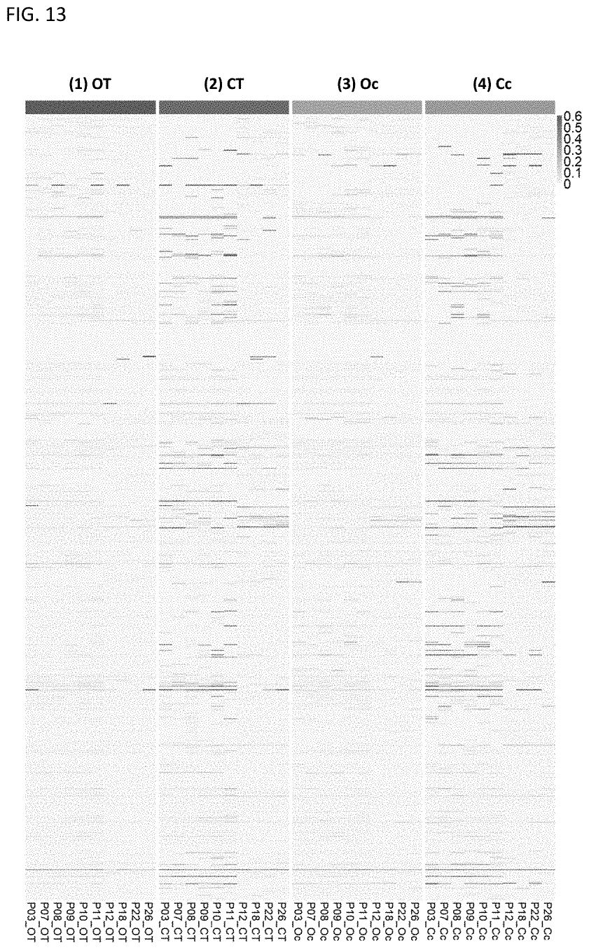

As further confirmation, the above enrichment step was applied to cancer patient samples (cancer tissue and blood from 10 non-small cell lung cancer patients with 9 in stage I and 1 in stage II), and the matching rate was confirmed by performing targeted NGS sequencing using general targeted NGS and the above enrichment process, respectively. In total, the landscape of 1,056 genomic variants were analyzed. On average, a tissue-blood mutation concordance rate of about 70% was confirmed. The represented heatmap showed that CRISPR enrichment resulted in increased overall correlation of mutation profiles between tissue and ctDNA (see FIG. 13). For individual patient, the genomic variants were classified into eight conditions (or categories) as shown in Table 6 provided below.

TABLE 6
Description of Criteria for Each of Categories #1-#7 and X

| | | Original | CE | Original | CE | |
|---|---|---|---|---|---|---|
| | | tissue | tissue | cfDNA | cfDNA | |
| | | samples | samples | samples | samples | |
| Category | Occurrences | (n = 10) | (n = 10) | (n = 10) | (n = 10) | Description |
| 1 | 739 | ◯ | ◯ | ◯ | ◯ | Variants detected in all samples. |
| 2 | 126 | ◯ | ◯ | X | X | Variants found only in tissues and not in |
| | | | | | | cfDNA. |
| 3 | 170 | X | X | ◯ | ◯ | Variants found only in cfDNA and not in |
| | | | | | | tissues. |
| 4 | 57 | X | ◯ | X | ◯ | Variants found in tissues and cfDNA |
| | | | | | | only after CRISPR enrichment. |
| 5 | 190 | X | ◯ | X | X | Variants found only in tissues after |
| | | | | | | CRISPR enrichment. |
| 6 | 198 | X | X | X | ◯ | Variants found only in cfDNA after |
| | | | | | | CRISPR enrichment. |
| 7 | 6,592 | X | X | X | X | Variants not detected in any samples. |
| X | 2,128 | | | | | Cases not falling under categories 1-7. |

◯, occurring; X, not occurring; CE, CRISPR-enriched.

Among the 8 conditions, Category 7 had the most mutations with 6592, and Categories 4, 5, and 6 corresponding to CRISPR-enriched variants were found with 57, 190, and 198, respectively. CRISPR enrichment resulted in increase of detecting pathogenic variants from 445 to 1480 cases out of 10560 total possible cases.

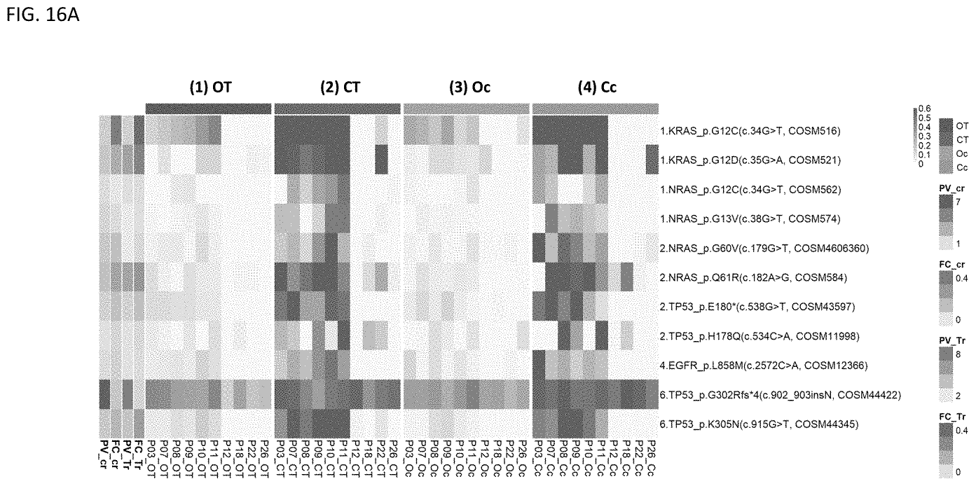

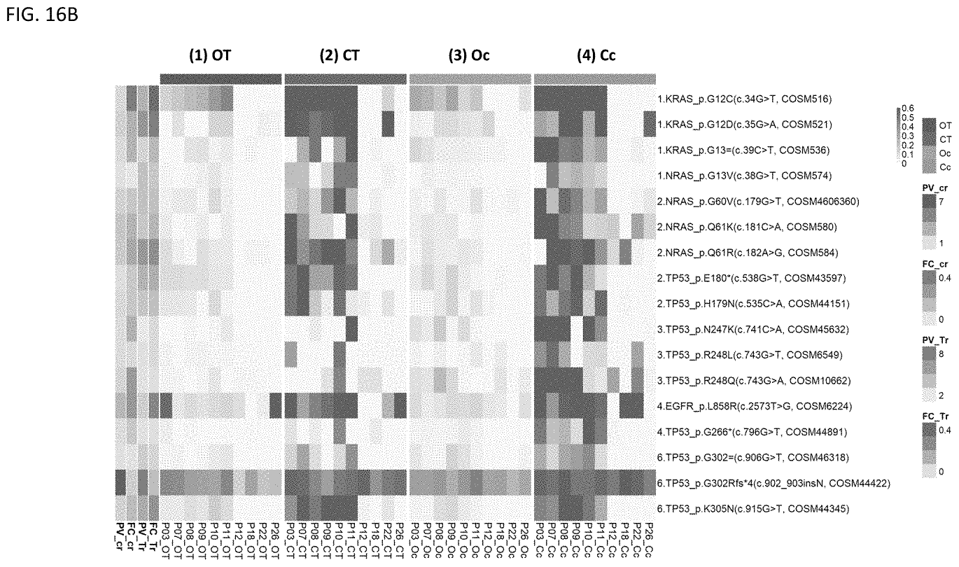

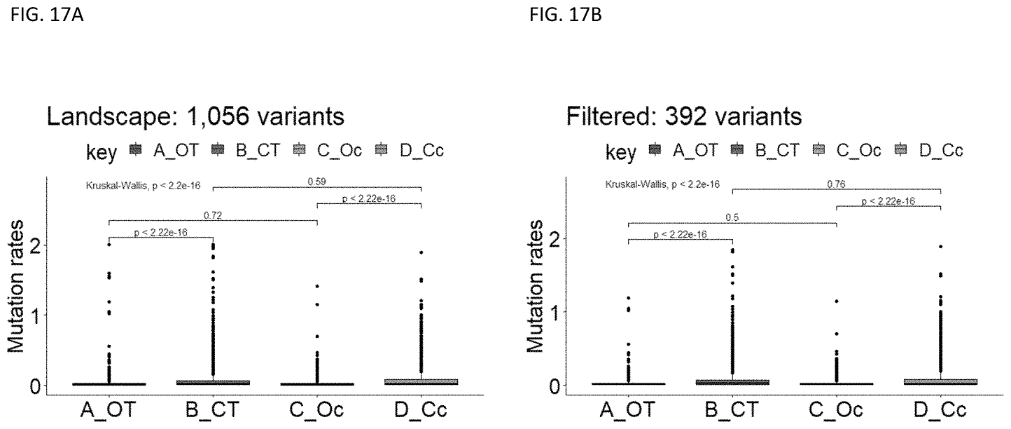

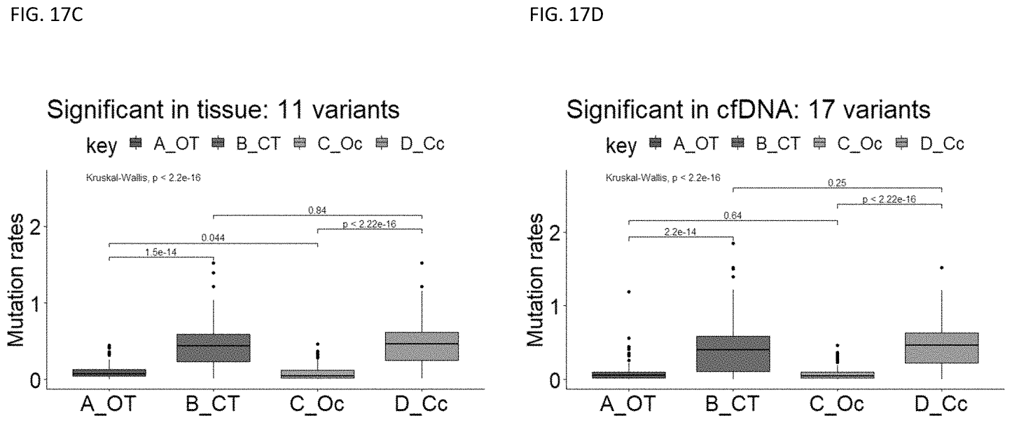

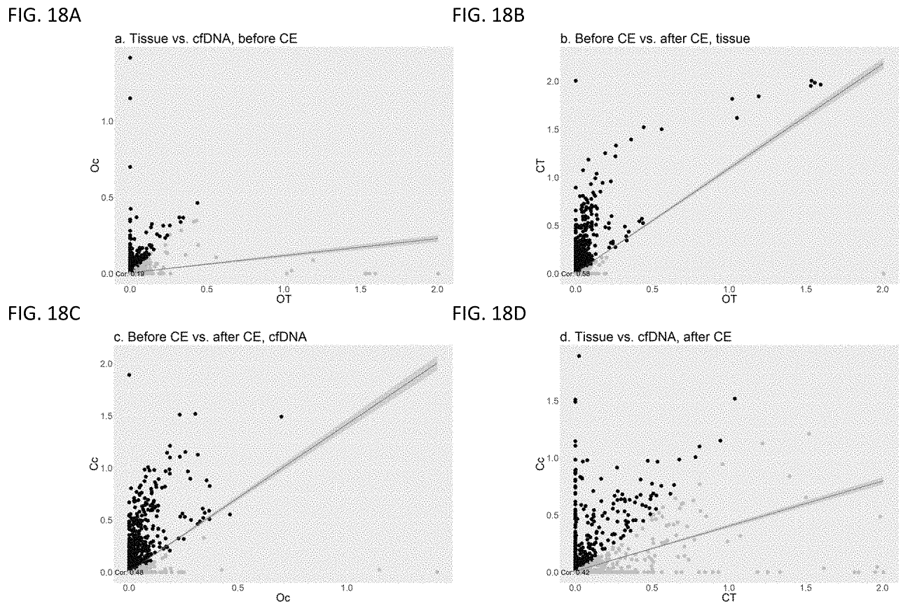

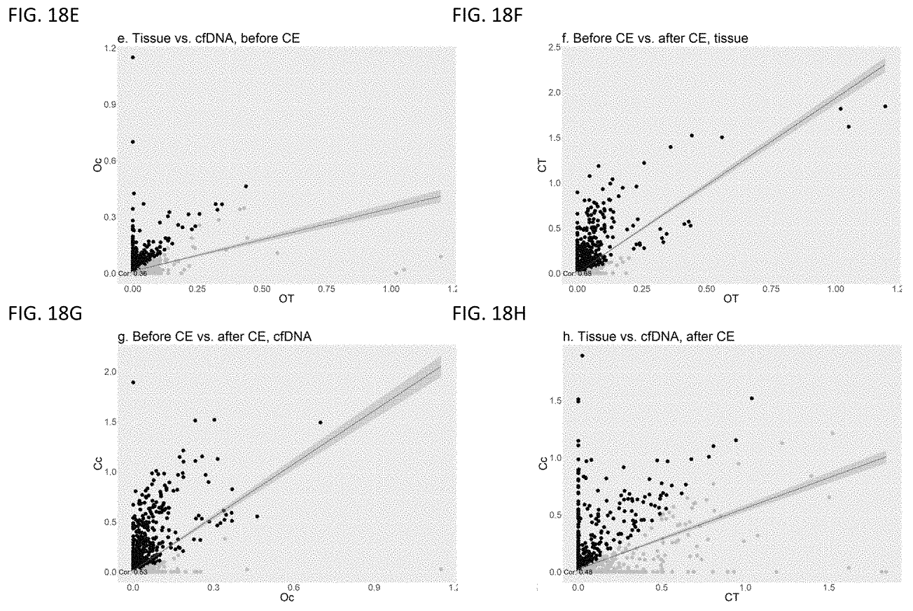

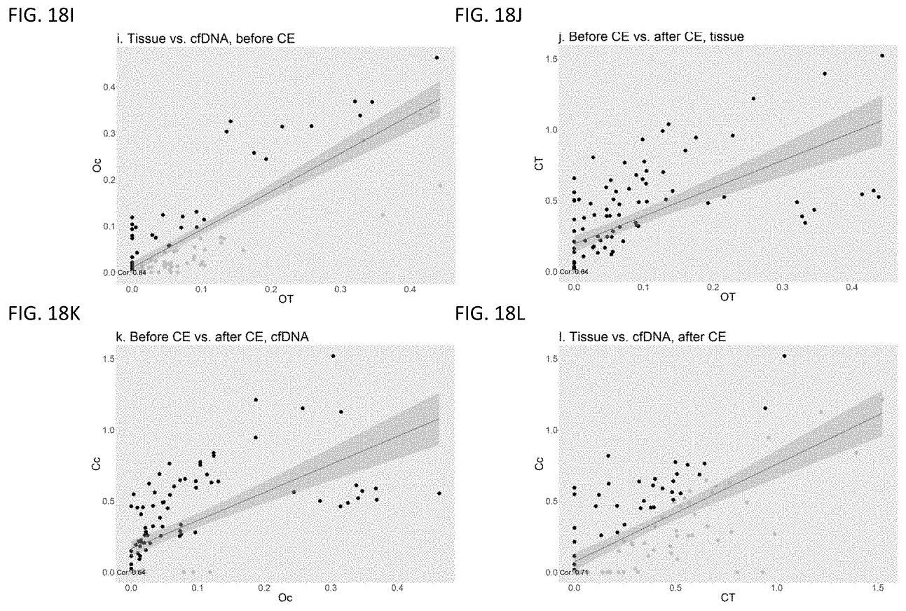

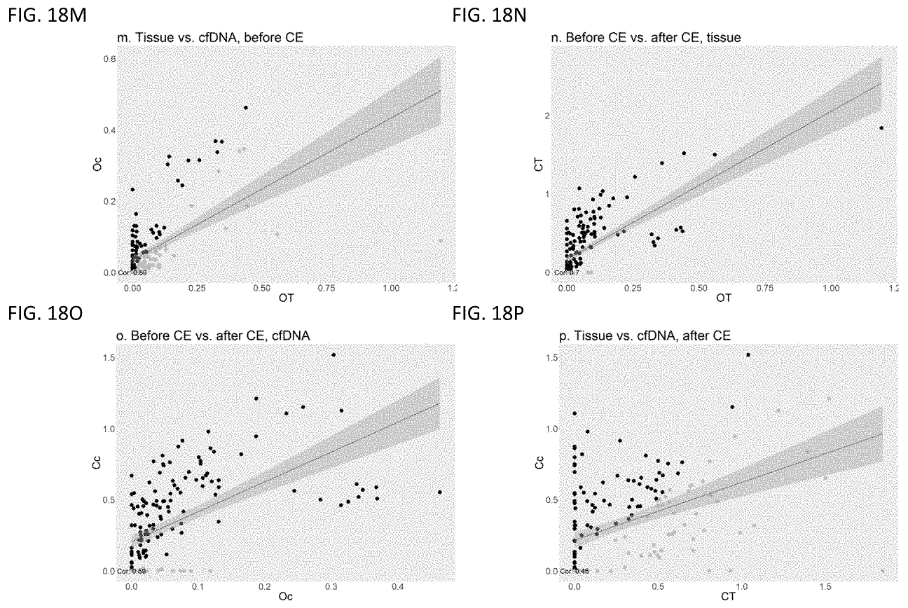

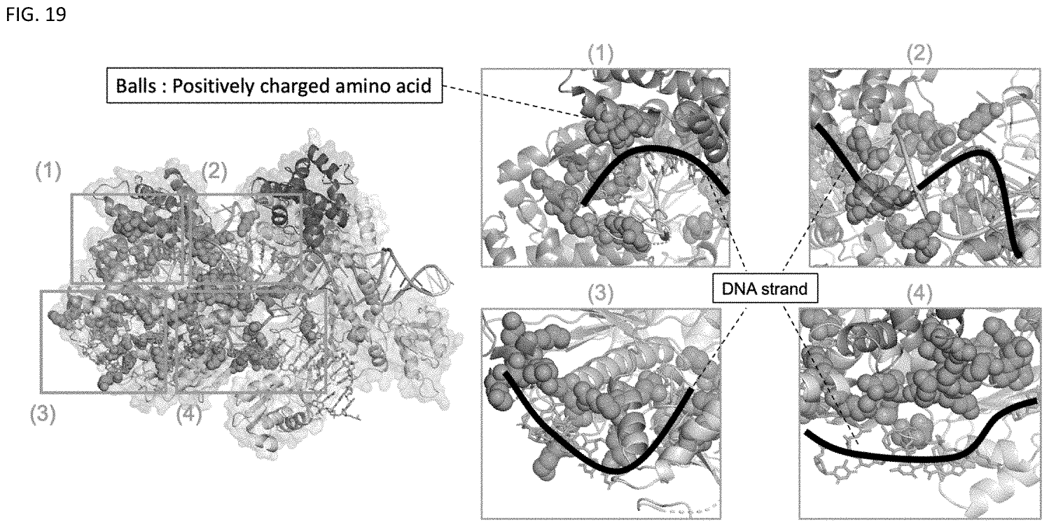

Next, the statistical significance and amount of change for mutations that were detected before and after enrichment were analyzed for each group of tissue and cfDNA. 11 and 17 variants satisfying p-value<0.05 and an absolute fold change>0.1, respectively, were analyzed in tissue DNA and cfDNA. As shown in FIGS. 16A-16B, 17A-17D, and 18A-18P, the enrichment resulted in statistically significant increase in the detection rates of the mutations.

In summary, the above results demonstrate that the Cas9 proteins described herein are capable of efficiently discriminating single-base mutations in all 20 target positions and inducing DNA cleavage only for targets with perfect base matches. The above results further demonstrate that the accuracy of FnCas9-AF was greater than high-precision eSpCas9 and SpCas9-HF. The engineered FnCas9-AF provided herein efficiently distinguished base changes in all PAM and non-PAM positions, and could be utilized for flexible sgRNA design in detecting mutations in circulating tumor DNA (ctDNA) from cancer cells.

TABLE 7
FnCas9 Variant Sequences

| Name | Sequence |
|---|---|
| FnCas9-K1189A Variant | MNFKILPIAIDLGVKNTGVFSAFYQKGTSLERLDNKNGKVYELSKDSYTLLMNNRTA |
| (SEQ ID NO: 2) (mutated | RRHQRRGIDRKQLVKRLFKLIWTEQLNLEWDKDTQQAISFLFNRRGFSFITDGYSPE |
| amino acid is bolded) | YLNIVPEQVKAILMDIFDDYNGEDDLDSYLKLATEQESKISEIYNKLMQKILEFKLM |
| | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDANYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R1241A Variant | MNFKILPIAIDLGVKNTGVFSAFYQKGTSLERLDNKNGKVYELSKDSYTLLMNNRTA |
| (SEQ ID NO: 3) (mutated | RRHQRRGIDRKQLVKRLFKLIWTEQLNLEWDKDTQQAISFLFNRRGFSFITDGYSPE |
| amino acid is bolded) | YLNIVPEQVKAILMDIFDDYNGEDDLDSYLKLATEQESKISEIYNKLMQKILEFKLM |
| | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTADGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9- | MNFKILPIAIDLGVKNTGVFSAFYQKGTSLERLDNKNGKVYELSKDSYTLLMNNRTA |
| K1189A + R1241A | RRHQRRGIDRKQLVKRLFKLIWTEQLNLEWDKDTQQAISFLFNRRGFSFITDGYSPE |
| Variant (“FnCas9-AF1”) | YLNIVPEQVKAILMDIFDDYNGEDDLDSYLKLATEQESKISEIYNKLMQKILEFKLM |
| (SEQ ID NO: 4) (mutated | KLCTDIKDDKVSTKTLKEITSYEFELLADYLANYSESLKTQKFSYTDKQGNLKELSY |
| amino acids are bolded) | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDANYSLYPLD |
| | KNTGEVFTKDIFSQIKITDNEFSDKKLVRKKAIEGFNTHRQMTADGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R721A + R1241A | MNFKILPIAIDLGVKNTGVFSAFYQKGTSLERLDNKNGKVYELSKDSYTLLMNNRTA |
| Variant | RRHQRRGIDRKQLVKRLFKLIWTEQLNLEWDKDTQQAISFLFNRRGFSFITDGYSPE |
| (SEQ ID NO: 5) (mutated | YLNIVPEQVKAILMDIFDDYNGEDDLDSYLKLATEQESKISEIYNKLMQKILEFKLM |
| amino acids are bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNAGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTADGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9- | MNFKILPIAIDLGVKNTGVFSAFYQKGTSLERLDNKNGKVYELSKDSYTLLMNNRTA |
| R785A + R1241A Variant | RRHQRRGIDRKQLVKRLFKLIWTEQLNLEWDKDTQQAISFLFNRRGFSFITDGYSPE |
| (SEQ ID NO: 6) (mutated | YLNIVPEQVKAILMDIFDDYNGEDDLDSYLKLATEQESKISETYNKLMQKILEFKLM |
| amino acids are bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDAKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTADGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9- | MNFKILPIAIDLGVKNTGVFSAFYQKGTSLERLDNKNGKVYELSKDSYTLLMNNRTA |
| R785A + K1189A + R1241A | RRHQRRGIDRKQLVKRLFKLIWTEQLNLEWDKDTQQAISFLFNRRGFSFITDGYSPE |
| Variant “FnCas9-AF2” | YLNIVPEQVKAILMDIFDDYNGEDDLDSYLKLATEQESKISEIYNKLMQKILEFKLM |
| (SEQ ID NO: 7) | KLCTDIKDDKVSTKTLKEITSYEFELLADYLANYSESLKTQKFSYTDKQGNLKELSY |
| (mutated amino acids are | YHHDKYNIQEFLKRHATINDRILDTLLTDDLDIWNFNFEKFDFDKNEEKLQNQEDKD |
| bolded) | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDAKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDANYSLYPLD |
| | KNTGEVFTKDIFSQIKITDNEFSDKKLVRKKAIEGFNTHRQMTADGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9- | MNFKILPIAIDLGVKNTGVFSAFYQKGTSLERLDNKNGKVYELSKDSYTLLMNNRTA |
| R721A + K1189A + R1241A | RRHQRRGIDRKQLVKRLFKLIWTEQLNLEWDKDTQQAISFLFNRRGFSFITDGYSPE |
| Variant (SEQ ID NO: 8) | YLNIVPEQVKAILMDIFDDYNGEDDLDSYLKLATEQESKISETYNKLMQKILEFKLM |
| (mutated amino acids | KLCTDIKDDKVSTKTLKEITSYEFELLADYLANYSESLKTQKFSYTDKQGNLKELSY |
| are bolded) | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNAGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDANYSLYPLD |
| | KNTGEVFTKDIFSQIKITDNEFSDKKLVRKKAIEGFNTHRQMTADGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9- | MNFKILPIAIDLGVKNTGVFSAFYQKGTSLERLDNKNGKVYELSKDSYTLLMNNRTA |
| K1189A + K1228A + R1241A | RRHQRRGIDRKQLVKRLFKLIWTEQLNLEWDKDTQQAISFLFNRRGFSFITDGYSPE |
| Variant (SEQ ID NO: 9) | YLNIVPEQVKAILMDIFDDYNGEDDLDSYLKLATEQESKISETYNKLMQKILEFKLM |
| (mutated amino | KLCTDIKDDKVSTKTLKEITSYEFELLADYLANYSESLKTQKFSYTDKQGNLKELSY |
| acids are bolded) | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNETYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDANYSLYPLD |
| | KNTGEVFTKDIFSQIKITDNEFSDKKLVRKAAIEGFNTHRQMTADGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYTENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K405A Variant | MNFKILPIAIDLGVKNTGVFSAFYQKGTSLERLDNKNGKVYELSKDSYTLLMNNRTA |
| (SEQ ID NO: 251) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVADQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNETYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R455A Variant | MNFKILPIAIDLGVKNTGVFSAFYQKGTSLERLDNKNGKVYELSKDSYTLLMNNRTA |
| (SEQ ID NO: 252) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNAK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNETYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K566A Variant | MNFKILPIAIDLGVKNTGVFSAFYQKGTSLERLDNKNGKVYELSKDSYTLLMNNRTA |
| (SEQ ID NO: 253) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQAASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYTENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGTYNETSNN |
| FnCas9-K578A Variant | MNFKILPIAIDLGVKNTGVFSAFYQKGTSLERLDNKNGKVYELSKDSYTLLMNNRTA |
| (SEQ ID NO: 254) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSAKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K664A Variant | MNFKILPIAIDLGVKNTGVFSAFYQKGTSLERLDNKNGKVYELSKDSYTLLMNNRTA |
| (SEQ ID NO: 255) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQARYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R721A Variant | MNFKILPIAIDLGVKNTGVFSAFYQKGTSLERLDNKNGKVYELSKDSYTLLMNNRTA |
| (SEQ ID NO: 256) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNAGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R785A Variant | MNFKILPIAIDLGVKNTGVFSAFYQKGTSLERLDNKNGKVYELSKDSYTLLMNNRTA |
| (SEQ ID NO: 257) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDAKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K786A Variant | MNFKILPIAIDLGVKNTGVFSAFYQKGTSLERLDNKNGKVYELSKDSYTLLMNNRTA |
| (SEQ ID NO: 258) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRAIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K789A Variant | MNFKILPIAIDLGVKNTGVFSAFYQKGTSLERLDNKNGKVYELSKDSYTLLMNNRTA |
| (SEQ ID NO: 259) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKAFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K914A Variant | MNFKILPIAIDLGVKNTGVFSAFYQKGTSLERLDNKNGKVYELSKDSYTLLMNNRTA |
| (SEQ ID NO: 260) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GASLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K917A Variant | MNFKILPIAIDLGVKNTGVFSAFYQKGTSLERLDNKNGKVYELSKDSYTLLMNNRTA |
| (SEQ ID NO: 261) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNETYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLADRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R919A Variant | MNFKILPIAIDLGVKNTGVFSAFYQKGTSLERLDNKNGKVYELSKDSYTLLMNNRTA |
| (SEQ ID NO: 262) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDARKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGTYNETSNN |
| FnCas9-K921A Variant | MNFKILPIAIDLGVKNTGVFSAFYQKGTSLERLDNKNGKVYELSKDSYTLLMNNRTA |
| (SEQ ID NO: 263) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRAKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K922A Variant | MNFKILPIAIDLGVKNTGVFSAFYQKGTSLERLDNKNGKVYELSKDSYTLLMNNRTA |
| (SEQ ID NO: 264) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKAALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R926A Variant | MNFKILPIAIDLGVKNTGVFSAFYQKGTSLERLDNKNGKVYELSKDSYTLLMNNRTA |
| (SEQ ID NO: 265) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALEAISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K934A variant | MNFKILPIAIDLGVKNTGVFSAFYQKGTSLERLDNKNGKVYELSKDSYTLLMNNRTA |
| (SEQ ID NO: 266) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENTFADKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K936A Variant | MNFKILPIAIDLGVKNTGVFSAFYQKGTSLERLDNKNGKVYELSKDSYTLLMNNRTA |
| (SEQ ID NO: 267) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDANNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R939A Variant | MNFKILPIAIDLGVKNTGVFSAFYQKGTSLERLDNKNGKVYELSKDSYTLLMNNRTA |
| (SEQ ID NO: 268) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | GKSLKDRRKKALERISPENIFKDKNNAIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K941A Variant | MNFKILPIAIDLGVKNTGVFSAFYQKGTSLERLDNKNGKVYELSKDSYTLLMNNRTA |
| (SEQ ID NO: 269) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIAEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K945A Variant | MNFKILPIAIDLGVKNTGVFSAFYQKGTSLERLDNKNGKVYELSKDSYTLLMNNRTA |
| (SEQ ID NO: 270) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAAGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R1137A Variant | MNFKILPIAIDLGVKNTGVFSAFYQKGTSLERLDNKNGKVYELSKDSYTLLMNNRTA |
| (SEQ ID NO: 271) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIAQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K1142A Variant | MNFKILPIAIDLGVKNTGVFSAFYQKGTSLERLDNKNGKVYELSKDSYTLLMNNRTA |
| (SEQ ID NO: 272) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EAVDSDIQAYAKGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K1152A Variant | MNFKILPIAIDLGVKNTGVFSAFYQKGTSLERLDNKNGKVYELSKDSYTLLMNNRTA |
| (SEQ ID NO: 273) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNETYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAAGDKPQASYSHLIDAMLAFCIAADEHRNDGSIGLEIDKNYSLYPLD |
| | KNTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K1198A Variant | MNFKILPIAIDLGVKNTGVFSAFYQKGTSLERLDNKNGKVYELSKDSYTLLMNNRTA |
| (SEQ ID NO: 274) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNETYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | ANTGEVFTKDIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K1206A Variant | MNFKILPIAIDLGVKNTGVFSAFYQKGTSLERLDNKNGKVYELSKDSYTLLMNNRTA |
| (SEQ ID NO: 275) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNETYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTADIFSQIKITDNEFSDKKLVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K1223A Variant | MNFKILPIAIDLGVKNTGVFSAFYQKGTSLERLDNKNGKVYELSKDSYTLLMNNRTA |
| (SEQ ID NO: 276) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKALVRKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-R1226A Variant | MNFKILPIAIDLGVKNTGVFSAFYQKGTSLERLDNKNGKVYELSKDSYTLLMNNRTA |
| (SEQ ID NO: 277) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNETYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSIYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVAKKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K1227A Variant | MNFKILPIAIDLGVKNTGVFSAFYQKGTSLERLDNKNGKVYELSKDSYTLLMNNRTA |
| (SEQ ID NO: 278) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISETYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRAKAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYTENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |
| FnCas9-K1228A Variant | MNFKILPIAIDLGVKNTGVFSAFYQKGTSLERLDNKNGKVYELSKDSYTLLMNNRTA |
| (SEQ ID NO: 279) | RRHQRRGIDRKQLVKRLFKLIWTEQLNLEWDKDTQQAISFLFNRRGFSFITDGYSPE |
| (mutated amino acid is | YLNIVPEQVKAILMDIFDDYNGEDDLDSYLKLATEQESKISEIYNKLMQKILEFKLM |
| bolded) | KLCTDIKDDKVSTKTLKEITSYEFELLADYLANYSESLKTQKFSYTDKQGNLKELSY |
| | YHHDKYNIQEFLKRHATINDRILDTLLTDDLDIWNFNFEKFDFDKNEEKLQNQEDKD |
| | HIQAHLHHFVFAVNKIKSEMASGGRHRSQYFQEITNVLDENNHQEGYLKNFCENLHN |
| | KKYSNLSVKNLVNLIGNLSNLELKPLRKYFNDKIHAKADHWDEQKFTETYCHWILGE |
| | WRVGVKDQDKKDGAKYSYKDLCNELKQKVTKAGLVDFLLELDPCRTIPPYLDNNNRK |
| | PPKCQSLILNPKFLDNQYPNWQQYLQELKKLQSIQNYLDSFETDLKVLKSSKDQPYF |
| | VEYKSSNQQIASGQRDYKDLDARILQFIFDRVKASDELLLNEIYFQAKKLKQKASSE |
| | LEKLESSKKLDEVIANSQLSQILKSQHTNGIFEQGTFLHLVCKYYKQRQRARDSRLY |
| | IMPEYRYDKKLHKYNNTGRFDDDNQLLTYCNHKPRQKRYQLLNDLAGVLQVSPNFLK |
| | DKIGSDDDLFISKWLVEHIRGFKKACEDSLKIQKDNRGLLNHKINIARNTKGKCEKE |
| | IFNLICKIEGSEDKKGNYKHGLAYELGVLLFGEPNEASKPEFDRKIKKFNSTYSFAQ |
| | IQQIAFAERKGNANTCAVCSADNAHRMQQIKITEPVEDNKDKIILSAKAQRLPAIPT |
| | RIVDGAVKKMATILAKNIVDDNWQNIKQVLSAKHQLHIPIITESNAFEFEPALADVK |
| | GKSLKDRRKKALERISPENIFKDKNNRIKEFAKGISAYSGANLTDGDFDGAKEELDH |
| | IIPRSHKKYGTLNDEANLICVTRGDNKNKGNRIFCLRDLADNYKLKQFETTDDLEIE |
| | KKIADTIWDANKKDFKFGNYRSFINLTPQEQKAFRHALFLADENPIKQAVIRAINNR |
| | NRTFVNGTQRYFAEVLANNIYLRAKKENLNTDKISFDYFGIPTIGNGRGIAEIRQLY |
| | EKVDSDIQAYAKGDKPQASYSHLIDAMLAFCIAADEHRNDGSIGLEIDKNYSLYPLD |
| | KNTGEVFTKDIFSQIKITDNEFSDKKLVRKAAIEGFNTHRQMTRDGIYAENYLPILI |
| | HKELNEVRKGYTWKNSEEIKIFKGKKYDIQQLNNLVYCLKFVDKPISIDIQISTLEE |
| | LRNILTTNNIAATAEYYYINLKTQKLHEYYIENYNTALGYKKYSKEMEFLRSLAYRS |
| | ERVKIKSIDDVKQVLDKDSNFIIGKITLPFKKEWQRLYREWQNTTIKDDYEFLKSFF |
| | NVKSITKLHKKVRKDFSLPISTNEGKFLVKRKTWDNNFIYQILNDSDSRADGTKPFI |
| | PAFDISKNEIVEAIIDSFTSKNIFWLPKNIELQKVDNKNIFAIDTSKWFEVETPSDL |
| | RDIGIATIQYKIDNNSRPKVRVKLDYVIDDDSKINYFMNHSLLKSRYPDKVLEILKQ |
| | STIIEFESSGFNKTIKEMLGMKLAGIYNETSNN |

TABLE 8
SpCas9 Variant Protein Sequences

| | |
|---|---|
| eSpCas9(1.1) (i.e., | MDKKYSIGLDIGTNSVGWAVITDEYKVPSKKFKVLGNTDRHSIKKNLIGALLFDSGE |
| comprises K848A, | TAEATRLKRTARRRYTRRKNRICYLQEIFSNEMAKVDDSFFHRLEESFLVEEDKKHE |
| K1003A, and R1060A) | RHPIFGNIVDEVAYHEKYPTIYHLRKKLVDSTDKADLRLIYLALAHMIKFRGHFLIE |
| (SEQ ID NO: 245) | GDLNPDNSDVDKLFIQLVQTYNQLFEENPINASGVDAKAILSARLSKSRRLENLIAQ |
| (mutated amino acids | LPGEKKNGLFGNLIALSLGLTPNFKSNFDLAEDAKLQLSKDTYDDDLDNLLAQIGDQ |
| are bolded) | YADLFLAAKNLSDAILLSDILRVNTEITKAPLSASMIKRYDEHHQDLTLLKALVRQQ |
| | LPEKYKEIFFDQSKNGYAGYIDGGASQEEFYKFIKPILEKMDGTEELLVKLNREDLL |
| | RKQRTFDNGSIPHQIHLGELHAILRRQEDFYPFLKDNREKIEKILTFRIPYYVGPLA |
| | RGNSRFAWMTRKSEETITPWNFEEVVDKGASAQSFIERMTNFDKNLPNEKVLPKHSL |
| | LYEYFTVYNELTKVKYVTEGMRKPAFLSGEQKKAIVDLLFKTNRKVTVKQLKEDYFK |
| | KIECFDSVEISGVEDRFNASLGTYHDLLKIIKDKDFLDNEENEDILEDIVLTLTLFE |
| | DREMIEERLKTYAHLFDDKVMKQLKRRRYTGWGRLSRKLINGIRDKQSGKTILDFLK |
| | SDGFANRNFMQLIHDDSLTFKEDIQKAQVSGQGDSLHEHIANLAGSPAIKKGILQTV |
| | KVVDELVKVMGRHKPENIVIEMARENQTTQKGQKNSRERMKRIEEGIKELGSQILKE |
| | HPVENTQLQNEKLYLYYLQNGRDMYVDQELDINRLSDYDVDHIVPQSFLADDSIDNK |
| | VLTRSDKNRGKSDNVPSEEVVKKMKNYWRQLLNAKLITQRKFDNLTKAERGGLSELD |
| | KAGFIKRQLVETRQITKHVAQILDSRMNTKYDENDKLIREVKVITLKSKLVSDFRKD |
| | FQFYKVREINNYHHAHDAYLNAVVGTALIKKYPALESEFVYGDYKVYDVRKMIAKSE |
| | QEIGKATAKYFFYSNIMNFFKTEITLANGEIRKAPLIETNGETGEIVWDKGRDFATV |
| | RKVLSMPQVNIVKKTEVQTGGFSKESILPKRNSDKLIARKKDWDPKKYGGFDSPTVA |
| | YSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENITH |
| | LFTLTNLGAPAAFKYFDTTIDRKRYTSTKEVLDATLIHQSITGLYETRIDLSQLGGD |
| eSpCas9(1.0) (i.e., | MDKKYSIGLDIGTNSVGWAVITDEYKVPSKKFKVLGNTDRHSIKKNLIGALLFDSGE |
| comprises K810A, | TAEATRLKRTARRRYTRRKNRICYLQEIFSNEMAKVDDSFFHRLEESFLVEEDKKHE |
| K1003A, and R1060A) | RHPIFGNIVDEVAYHEKYPTIYHLRKKLVDSTDKADLRLIYLALAHMIKFRGHFLIE |
| (SEQ ID NO: 246) | GDLNPDNSDVDKLFIQLVQTYNQLFEENPINASGVDAKAILSARLSKSRRLENLIAQ |
| (mutated amino acids | LPGEKKNGLFGNLIALSLGLTPNFKSNFDLAEDAKLQLSKDTYDDDLDNLLAQIGDQ |
| are bolded) | YADLFLAAKNLSDAILLSDILRVNTEITKAPLSASMIKRYDEHHQDLTLLKALVRQQ |
| | LPEKYKEIFFDQSKNGYAGYIDGGASQEEFYKFIKPILEKMDGTEELLVKLNREDLL |
| | RKQRTFDNGSIPHQIHLGELHAILRRQEDFYPFLKDNREKIEKILTFRIPYYVGPLA |
| | RGNSRFAWMTRKSEETITPWNFEEVVDKGASAQSFIERMTNFDKNLPNEKVLPKHSL |
| | LYEYFTVYNELTKVKYVTEGMRKPAFLSGEQKKAIVDLLFKTNRKVTVKQLKEDYFK |
| | KIECFDSVEISGVEDRFNASLGTYHDLLKIIKDKDFLDNEENEDILEDIVLTLTLFE |
| | DREMIEERLKTYAHLFDDKVMKQLKRRRYTGWGRLSRKLINGIRDKQSGKTILDFLK |
| | SDGFANRNFMQLIHDDSLTFKEDIQKAQVSGQGDSLHEHIANLAGSPAIKKGILQTV |
| | KVVDELVKVMGRHKPENIVIEMARENQTTQKGQKNSRERMKRIEEGIKELGSQILKE |
| | HPVENTQLQNEALYLYYLQNGRDMYVDQELDINRLSDYDVDHIVPQSFLKDDSIDNK |
| | VLTRSDKNRGKSDNVPSEEVVKKMKNYWRQLLNAKLITQRKFDNLTKAERGGLSELD |
| | KAGFIKRQLVETRQITKHVAQILDSRMNTKYDENDKLIREVKVITLKSKLVSDFRKD |
| | FQFYKVREINNYHHAHDAYLNAVVGTALIKKYPALESEFVYGDYKVYDVRKMIAKSE |
| | QEIGKATAKYFFYSNIMNFFKTEITLANGEIRKAPLIETNGETGEIVWDKGRDFATV |
| | RKVLSMPQVNIVKKTEVQTGGFSKESILPKRNSDKLIARKKDWDPKKYGGFDSPTVA |
| | YSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIH |
| | LFTLTNLGAPAAFKYFDTTIDRKRYTSTKEVLDATLIHQSITGLYETRIDLSQLGGD |
| SpCas9-HF1 (i.e., | MDKKYSIGLDIGTNSVGWAVITDEYKVPSKKFKVLGNTDRHSIKKNLIGALLFDSGE |
| comprises N497A, | TAEATRLKRTARRRYTRRKNRICYLQEIFSNEMAKVDDSFFHRLEESFLVEEDKKHE |
| R661A, Q695A, and | RHPIFGNIVDEVAYHEKYPTIYHLRKKLVDSTDKADLRLIYLALAHMIKFRGHFLIE |
| Q926A) (SEQ ID NO: | GDLNPDNSDVDKLFIQLVQTYNQLFEENPINASGVDAKAILSARLSKSRRLENLIAQ |
| 247) | LPGEKKNGLFGNLIALSLGLTPNFKSNFDLAEDAKLQLSKDTYDDDLDNLLAQIGDQ |
| | YADLFLAAKNLSDAILLSDILRVNTEITKAPLSASMIKRYDEHHQDLTLLKALVRQQ |
| | LPEKYKEIFFDQSKNGYAGYIDGGASQEEFYKFIKPILEKMDGTEELLVKLNREDLL |
| | RKQRTFDNGSIPHQIHLGELHAILRRQEDFYPFLKDNREKIEKILTFRIPYYVGPLA |
| | RGNSRFAWMTRKSEETITPWNFEEVVDKGASAQSFIERMTAFDKNLPNEKVLPKHSL |
| | LYEYFTVYNELTKVKYVTEGMRKPAFLSGEQKKAIVDLLFKTNRKVTVKQLKEDYFK |
| | KIECFDSVEISGVEDRFNASLGTYHDLLKIIKDKDFLDNEENEDILEDIVLTLTLFE |
| | DREMIEERLKTYAHLFDDKVMKQLKRRRYTGWGALSRKLINGIRDKQSGKTILDFLK |
| | SDGFANRNFMALIHDDSLTFKEDIQKAQVSGQGDSLHEHIANLAGSPAIKKGILQTV |
| | KVVDELVKVMGRHKPENIVIEMARENQTTQKGQKNSRERMKRIEEGIKELGSQILKE |
| | HPVENTQLQNEKLYLYYLQNGRDMYVDQELDINRLSDYDVDHIVPQSFLKDDSIDNK |
| | VLTRSDKNRGKSDNVPSEEVVKKMKNYWRQLLNAKLITQRKFDNLTKAERGGLSELD |
| | KAGFIKRQLVETRAITKHVAQILDSRMNTKYDENDKLIREVKVITLKSKLVSDFRKD |
| | FQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATV |
| | RKVLSMPQVNIVKKTEVQTGGFSKESILPKRNSDKLIARKKDWDPKKYGGFDSPTVA |
| | YSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENITH |
| | LFTLTNLGAPAAFKYFDTTIDRKRYTSTKEVLDATLIHQSITGLYETRIDLSQLGGD |
| SpCas9-HF4 (i.e., | MDKKYSIGLDIGTNSVGWAVITDEYKVPSKKFKVLGNTDRHSIKKNLIGALLFDSGE |
| comprises Y450A, | TAEATRLKRTARRRYTRRKNRICYLQEIFSNEMAKVDDSFFHRLEESFLVEEDKKHE |
| N497A, R661A, Q695A, | RHPIFGNIVDEVAYHEKYPTIYHLRKKLVDSTDKADLRLIYLALAHMIKFRGHFLIE |
| and Q926A) (SEQ ID | GDLNPDNSDVDKLFIQLVQTYNQLFEENPINASGVDAKAILSARLSKSRRLENLIAQ |
| NO: 248) | LPGEKKNGLFGNLIALSLGLTPNFKSNFDLAEDAKLQLSKDTYDDDLDNLLAQIGDQ |
| | YADLFLAAKNLSDAILLSDILRVNTEITKAPLSASMIKRYDEHHQDLTLLKALVRQQ |
| | LPEKYKEIFFDQSKNGYAGYIDGGASQEEFYKFIKPILEKMDGTEELLVKLNREDLL |
| | RKQRTFDNGSIPHQIHLGELHAILRRQEDFYPFLKDNREKIEKILTFRIPAYVGPLA |
| | RGNSRFAWMTRKSEETITPWNFEEVVDKGASAQSFIERMTAFDKNLPNEKVLPKHSL |
| | LYEYFTVYNELTKVKYVTEGMRKPAFLSGEQKKAIVDLLFKTNRKVTVKQLKEDYFK |
| | KIECFDSVEISGVEDRFNASLGTYHDLLKIIKDKDFLDNEENEDILEDIVLTLTLFE |
| | DREMIEERLKTYAHLFDDKVMKQLKRRRYTGWGALSRKLINGIRDKQSGKTILDFLK |
| | SDGFANRNFMALIHDDSLTFKEDIQKAQVSGQGDSLHEHIANLAGSPAIKKGILQTV |
| | KVVDELVKVMGRHKPENIVIEMARENQTTQKGQKNSRERMKRIEEGIKELGSQILKE |
| | HPVENTQLQNEKLYLYYLQNGRDMYVDQELDINRLSDYDVDHIVPQSFLKDDSIDNK |
| | VLTRSDKNRGKSDNVPSEEVVKKMKNYWRQLLNAKLITQRKFDNLTKAERGGLSELD |
| | KAGFIKRQLVETRAITKHVAQILDSRMNTKYDENDKLIREVKVITLKSKLVSDFRKD |
| | FQFYKVREINNYHHAHDAYLNAVVGTALIKKYPKLESEFVYGDYKVYDVRKMIAKSE |
| | QEIGKATAKYFFYSNIMNFFKTEITLANGEIRKRPLIETNGETGEIVWDKGRDFATV |
| | RKVLSMPQVNIVKKTEVQTGGFSKESILPKRNSDKLIARKKDWDPKKYGGFDSPTVA |
| | YSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIH |
| | LFTLTNLGAPAAFKYFDTTIDRKRYTSTKEVLDATLIHQSITGLYETRIDLSQLGGD |

1. A Cas9 protein comprising a cavity domain that comprises a plurality of positively-charged amino acids, wherein at least one of the plurality of positively charged amino acids is modified (“amino acid modification”) compared to a corresponding wild-type Cas9 protein, and wherein the amino acid modification is capable of increasing the specificity of the Cas9 protein.

2. (canceled)

3. The Cas9 protein of claim 1, which comprises the amino acid sequence of SEQ ID NO: 1, and wherein the amino acid modification is at one or more of the following residues of SEQ ID NO: 1: K405, R455, K546, K561, K562, K564, K566, K578, K579, R618, R622, K664, R721, R785, R786, R788, K789, R807, R808, R849, R856, K914, K917, R919, K921, K922, R926, K934, K936, R939, K941, K945, R1047, R1131, R1137, K1142, K1152, K1155, R1178, K1189, K1198, K1206, K1213, K1223, R1226, K1227, K1228, R1241, or a combination thereof.

4. The Cas9 protein of claim 3, wherein the amino acid modification is at residue R785, K1189, R1241, or a combination thereof.

5. The Cas9 protein of claim 3, or wherein the amino acid modification is at residues: (a) K1189 and R1241 of SEQ ID NO: 1; (b) R721 and R1241 of SEQ ID NO: 1; (c) R785 and R1241 of SEQ ID NO: 1; (d) R785, K1189, and R1241 of SEQ ID NO: 1; (e) R721, K1189, and R1241 of SEQ ID NO: 1; or (f) K1189, K1228, and R1241 of SEQ ID NO: 1.

6-12. (canceled)

13. The Cas9 protein claim 1, wherein the amino acid modification comprises an alanine substitution.

14. The Cas9 protein of claim 1, which comprises, consists of, or consists essentially of:
(a) the amino acid sequence set forth in SEQ ID NO: 2;
(b) the amino acid sequence set forth in SEQ ID NO: 3;
(c) the amino acid sequence set forth in SEQ ID NO: 4;
(d) the amino acid sequence set forth in SEQ ID NO: 5;
(e) the amino acid sequence set forth in SEQ ID NO: 6;
(f) the amino acid sequence set forth in SEQ ID NO: 7;
(g) the amino acid sequence set forth in SEQ ID NO: 8;
(h) the amino acid sequence set forth in SEQ ID NO: 9.

15-21. (canceled)

22. A composition comprising the Cas9 protein of claim 1.

23-24. (canceled)

25. An isolated polynucleotide encoding the Cas9 protein of claim 1.

26. A vector comprising the isolated polynucleotide of claim 25.

27. A cell comprising the vector of claim 26.

28-30. (canceled)

31. A method of enriching for a first nucleotide sequence in a biological sample, which comprises the first nucleotide sequence and a second nucleotide sequence, the method comprising contacting the biological sample with the Cas9 protein of claim 1, wherein the first nucleotide sequence and the second nucleotide sequence are different, and wherein the Cas9 protein is capable of cleaving the second nucleotide sequence but not the first nucleotide sequence.

32-34. (canceled)

35. A method of measuring the amount of a first nucleotide sequence in a biological sample comprising the first nucleotide sequence and one or more additional nucleotide sequences which differ from the first nucleotide sequence, the method comprising contacting the biological sample with the Cas9 protein of claim 1, and wherein the contacting reduces the amount of one or more additional nucleotide sequences present in the biological sample.

36. The method of claim 31, wherein the first nucleotide sequence comprises a mutation and the second nucleotide sequence does not comprise the mutation.

37-41. (canceled)

42. The method of claim 36, wherein the mutation is within: (i) a target site to which a guide polynucleotide binds, (ii) a protospacer adjacent motif (PAM), or (iii) both (i) and (ii).

43-45. (canceled)

46. A method of diagnosing a disease in a subject in need thereof, the method comprising detecting whether the amount of a nucleotide sequence, which comprises a mutation that is associated with the disease, is increased in a biological sample obtained from the subject compared to a corresponding amount present in a reference sample (e.g., biological sample obtained from a subject who does not suffer from the disease), wherein prior to the detecting, the biological sample was contacted with the Cas9 protein of claim 1.

47-49. (canceled)

50. The method of claim 46, wherein the disease comprises a cancer, hematologic disease, neurodegenerative/neurologic disease, infectious disease, rheumatoid disease, allergic disease, psychiatric disease, optical disease, endocrinologic disease, congenital disease, cardiovascular disease, pulmonary disease, nephrologic disease, gastrologic disease, hepatologic disease, or a combination thereof.

51-52. (canceled)

53. A method of reducing an occurrence of an off-target cleavage of a nucleic acid sequence during a CRISPR-based gene editing, comprising contacting the nucleic acid sequence with a complex comprising a Cas9 protein and a guide polynucleotide, wherein the Cas9 protein comprises an amino acid modification which is capable of increasing the specificity of the Cas9 protein, thereby reducing the occurrence of an off-target cleavage.

54-57. (canceled)

58. A method of increasing a specificity of a Cas9 protein comprising modifying at least one amino acid residue of the Cas9 protein, wherein the at least one amino acid residue is capable of interacting with a backbone phosphate of a DNA sequence.

59. The method of claim 58, wherein the at least one amino acid residue which is modified comprises residue K405, R455, K546, K561, K562, K564, K566, K578, K579, R618, R622, K664, R721, R785, K786, K788, K789, R807, K808, R849, R856, K914, K917, R919, R920, K921, K922, R926, K934, K936, R939, K941, K945, R1047, R1131, R1137, K1142, K1152, K1155, R1178, K1189, K1198, K1206, K1213, K1223, R1226, K1227, K1228, R1241, or a combination thereof, corresponding to the amino acid sequence set forth in SEQ ID NO: 1.

60-70. (canceled)

71. A method of genetically modifying a cell, comprising contacting a cell with the Cas9 protein of claim 1, wherein the contacting results in the modification of one or more DNA sequences of the cell.

72. (canceled)


SEQUENCE LISTING (296 sequences)

SEQ ID NO: 1 - protein, 1629 aa, Francisella tularensis
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 2 - protein, 1368 aa, synthetic construct [sequence omitted]

SEQ ID NO: 3 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTADGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 4 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDANYSLYPLDKNTGEVFTKDIFSQIKITDNEFSDKKLVRKKAIEGFNTHRQMTADGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 5 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNAGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTADGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 6 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDAKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTADGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 7 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDAKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDANYSLYPLDKNTGEVFTKDIFSQIKITDNEFSDKKLVRKKAIEGFNTHRQMTADGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 8 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNAGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDANYSLYPLDKNTGEVFTKDIFSQIKITDNEFSDKKLVRKKAIEGFNTHRQMTADGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 9 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDANYSLYPLDKNTGEVFTKDIFSQIKITDNEFSDKKLVRKAAIEGFNTHRQMTADGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 10 - DNA, 24 bp, synthetic construct
  source 1..24: mol_type = other DNA
acttacttctgacaacgatcggag

SEQ ID NO: 11 - DNA, 21 bp, synthetic construct
  source 1..21: mol_type = other DNA
gggaataagggcgacacggaa

SEQ ID NO: 12 - DNA, 55 bp, synthetic construct
  source 1..55: mol_type = other DNA
acactctttccctacacgacgctcttccgatctgagaaagttaaaattcccgtcg

SEQ ID NO: 13 - DNA, 63 bp, synthetic construct
  source 1..63: mol_type = other DNA
gtgactggagttcagacgtgtgctcttccgatctcacatcgaggatttccttgttgcctttcg

SEQ ID NO: 14 - DNA, 60 bp, synthetic construct
  source 1..60: mol_type = other DNA
acactctttccctacacgacgctcttccgatctccctccctccaggaagcctacctgatg

SEQ ID NO: 15 - DNA, 53 bp, synthetic construct
  source 1..53: mol_type = other DNA
gtgactggagttcagacgtgtgctcttccgatctggtggaggtgaggcagatg

SEQ ID NO: 16 - DNA, 55 bp, synthetic construct
  source 1..55: mol_type = other DNA
acactctttccctacacgacgctcttccgatctctcacctccaccgtgccgctca

SEQ ID NO: 17 - DNA, 58 bp, synthetic construct
  source 1..58: mol_type = other DNA
gtgactggagttcagacgtgtgctcttccgatctcacaccagttgagcaggtactggg

SEQ ID NO: 18 - DNA, 50 bp, synthetic construct
  source 1..50: mol_type = other DNA
acactctttccctacacgacgctcttccgatctcttggtgcaccgcgacc

SEQ ID NO: 19 - DNA, 58 bp, synthetic construct
  source 1..58: mol_type = other DNA
gtgactggagttcagacgtgtgctcttccgatctttctcttccgcacccagccgtttg

SEQ ID NO: 20 - DNA, 51 bp, synthetic construct
  source 1..51: mol_type = other DNA
acactctttccctacacgacgctcttccgatctcgtcaaggcactcttgcc

SEQ ID NO: 21 - DNA, 62 bp, synthetic construct
  source 1..62: mol_type = other DNA
gtgactggagttcagacgtgtgctcttccgatcttgctgaaaatgactgaatataaacttgt

SEQ ID NO: 22 - DNA, 67 bp, synthetic construct
  source 1..67: mol_type = other DNA
acactctttccctacacgacgctcttccgatctctgtatttatttcagtgttacttacctgtcttgt

SEQ ID NO: 23 - DNA, 64 bp, synthetic construct
  source 1..64: mol_type = other DNA
gtgactggagttcagacgtgtgctcttccgatctgctcaggacttagcaagaagttatggaatt

SEQ ID NO: 24 - DNA, 52 bp, synthetic construct
  source 1..52: mol_type = other DNA
acactctttccctacacgacgctcttccgatctattgtcagtgcgcttttcc

SEQ ID NO: 25 - DNA, 55 bp, synthetic construct
  source 1..55: mol_type = other DNA
gtgactggagttcagacgtgtgctcttccgatctttgctggtgtgaaatgactga

SEQ ID NO: 26 - DNA, 58 bp, synthetic construct
  source 1..58: mol_type = other DNA
acactctttccctacacgacgctcttccgatcttggtctctcatggcactgtcctctt

SEQ ID NO: 27 - DNA, 55 bp, synthetic construct
  source 1..55: mol_type = other DNA
gtgactggagttcagacgtgtgctcttccgatctatggtgaaacctgtttgttgg

SEQ ID NO: 28 - DNA, 53 bp, synthetic construct
  source 1..53: mol_type = other DNA
acactctttccctacacgacgctcttccgatcggttttctgggaagggacaga

SEQ ID NO: 29 - DNA, 53 bp, synthetic construct
  source 1..53: mol_type = other DNA
gtgactggagttcagacgtgtgctcttccgatctcaccagcagctcctacacc

SEQ ID NO: 30 - DNA, 58 bp, synthetic construct
  source 1..58: mol_type = other DNA
acactctttccctacacgacgctcttccgatctcaccatcgctatctgagcaccgctc

SEQ ID NO: 31 - DNA, 56 bp, synthetic construct
  source 1..56: mol_type = other DNA
gtgactggagttcagacgtgtgctcttccgatcttctacaagcagtcacagcacat

SEQ ID NO: 32 - DNA, 51 bp, synthetic construct
  source 1..51: mol_type = other DNA
acactctttccctacacgacgctcttccgatctgtggcaagtggctcctga

SEQ ID NO: 33 - DNA, 64 bp, synthetic construct
  source 1..64: mol_type = other DNA
gtgactggagttcagacgtgtgctcttccgatcttgtaacagttcctgcatgggccgcatgaac

SEQ ID NO: 34 - DNA, 60 bp, synthetic construct
  source 1..60: mol_type = other DNA
acactctttccctacacgacgctcttccgatctacaaacacgcacctcaaacctgttccg

SEQ ID NO: 35 - DNA, 56 bp, synthetic construct
  source 1..56: mol_type = other DNA
gtgactggagttcagacgtgtgctcttccgatcttgcctcttgcttctcttttcct

SEQ ID NO: 36 - DNA, 52 bp, synthetic construct
  source 1..52: mol_type = other DNA
acactctttccctacacgacgctcttccgatctgtgaggctcccctttcttg

SEQ ID NO: 37 - DNA, 53 bp, synthetic construct
  source 1..53: mol_type = other DNA
gtgactggagttcagacgtgtgctcttccgatctcgtgtttgtgcctgtcctg

SEQ ID NO: 38 - DNA, 60 bp, synthetic construct
  source 1..60: mol_type = other DNA
acactctttccctacacgacgctcttccgatctcaccgcttcttgtcctgcttccttacc

SEQ ID NO: 39 - DNA, 52 bp, synthetic construct
  source 1..52: mol_type = other DNA
gtgactggagttcagacgtgtgctcttccgatctaaaggggagcctcaccac

SEQ ID NO: 40 - DNA, 53 bp, synthetic construct
  source 1..53: mol_type = other DNA
acactctttccctacacgacgctcttccgatcttgagcaagaggctttggagt

SEQ ID NO: 41 - DNA, 55 bp, synthetic construct
  source 1..55: mol_type = other DNA
gtgactggagttcagacgtgtgctcttccgatctccaatccatttttgttgtcca

SEQ ID NO: 42 - DNA, 60 bp, synthetic construct
  source 1..60: mol_type = other DNA
acactctttccctacacgacgctcttccgatcttccacacaattaaacagcctgcattga

SEQ ID NO: 43 - DNA, 58 bp, synthetic construct
  source 1..58: mol_type = other DNA
gtgactggagttcagacgtgtgctcttccgatcttggaatccagagtgagctttcatt

SEQ ID NO: 44 - DNA, 61 bp, synthetic construct
  source 1..61: mol_type = other DNA
acactctttccctacacgacgctcttccgatcttcatgaagacctcacagtaaaaataggt

SEQ ID NO: 45 - DNA, 57 bp, synthetic construct
  source 1..57: mol_type = other DNA
gtgactggagttcagacgtgtgctcttccgatctctgatgggacccactccaccgag

SEQ ID NO: 46 - DNA, 56 bp, synthetic construct
  source 1..56: mol_type = other DNA
acactctttccctacacgacgctcttccgatctgtgaaatctcgatggagtgggtc

SEQ ID NO: 47 - DNA, 63 bp, synthetic construct
  source 1..63: mol_type = other DNA
gtgactggagttcagacgtgtgctcttccgatcttggaaaaatagcctcaattcttaccatcc

SEQ ID NO: 48 - RNA, 20 nt, synthetic construct
  source 1..20: mol_type = other RNA
gtagttggagctggtggcgt

SEQ ID NO: 49 - RNA, 20 nt, synthetic construct
  source 1..20: mol_type = other RNA
gtagttggagctggtggcga

SEQ ID NO: 50 - RNA, 20 nt, synthetic construct
  source 1..20: mol_type = other RNA
gtagttggagctggtggcct

SEQ ID NO: 51 - RNA, 20 nt, synthetic construct
  source 1..20: mol_type = other RNA
gtagttggagctggtggggt

SEQ ID NO: 52 - RNA, 20 nt, synthetic construct
  source 1..20: mol_type = other RNA
gtagttggagctggtgccgt

SEQ ID NO: 53 - RNA, 20 nt, synthetic construct
  source 1..20: mol_type = other RNA
gtagttggagctggtcgcgt

SEQ ID NO: 54 - RNA, 20 nt, synthetic construct
  source 1..20: mol_type = other RNA
gtagttggagctggaggcgt

SEQ ID NO: 55 - RNA, 20 nt, synthetic construct
  source 1..20: mol_type = other RNA
gtagttggagctgctggcgt

SEQ ID NO: 56 - RNA, 20 nt, synthetic construct
  source 1..20: mol_type = other RNA
gtagttggagctcgtggcgt

SEQ ID NO: 57 - RNA, 20 nt, synthetic construct
  source 1..20: mol_type = other RNA
gtagttggagcaggtggcgt

SEQ ID NO: 58 - RNA, 20 nt, synthetic construct
  source 1..20: mol_type = other RNA
gtagttggaggtggtggcgt

SEQ ID NO: 59 - RNA, 20 nt, synthetic construct
  source 1..20: mol_type = other RNA
gtagttggacctggtggcgt

SEQ ID NO: 60 - RNA, 20 nt, synthetic construct
  source 1..20: mol_type = other RNA
gtagttggtgctggtggcgt

SEQ ID NO: 61 - RNA, 20 nt, synthetic construct
  source 1..20: mol_type = other RNA
gtagttgcagctggtggcgt

SEQ ID NO: 62 - RNA, 20 nt, synthetic construct
  source 1..20: mol_type = other RNA
gtagttcgagctggtggcgt

SEQ ID NO: 63 - RNA, 20 nt, synthetic construct
  source 1..20: mol_type = other RNA
gtagtaggagctggtggcgt

SEQ ID NO: 64 - RNA, 20 nt, synthetic construct
  source 1..20: mol_type = other RNA
gtagatggagctggtggcgt

SEQ ID NO: 65 - RNA, 20 nt, synthetic construct
  source 1..20: mol_type = other RNA
gtacttggagctggtggcgt

SEQ ID NO: 66 - RNA, 20 nt, synthetic construct
  source 1..20: mol_type = other RNA
gttgttggagctggtggcgt

SEQ ID NO: 67 - RNA, 20 nt, synthetic construct
  source 1..20: mol_type = other RNA
gaagttggagctggtggcgt

SEQ ID NO: 68 - RNA, 20 nt, synthetic construct
  source 1..20: mol_type = other RNA
ctagttggagctggtggcgt

SEQ ID NO: 69 - RNA, 20 nt, synthetic construct
  source 1..20: mol_type = other RNA
gtagttggagctggtggcgg

SEQ ID NO: 70 - RNA, 20 nt, synthetic construct
  source 1..20: mol_type = other RNA
gtagttggagctggtggcgc

SEQ ID NO: 71 - RNA, 20 nt, synthetic construct
  source 1..20: mol_type = other RNA
gtagttggagctggtggcat

SEQ ID NO: 72 - RNA, 20 nt, synthetic construct
  source 1..20: mol_type = other RNA
gtagttggagctggtggctt

SEQ ID NO: 73 - RNA, 20 nt, synthetic construct
  source 1..20: mol_type = other RNA
gtagttggagctggtggagt

SEQ ID NO: 74 - RNA, 20 nt, synthetic construct
  source 1..20: mol_type = other RNA
gtagttggagctggtggtgt

SEQ ID NO: 75 - RNA, 20 nt, synthetic construct
  source 1..20: mol_type = other RNA
gtagttggagctggtgacgt

SEQ ID NO: 76 - RNA, 20 nt, synthetic construct
  source 1..20: mol_type = other RNA
gtagttggagctggtgtcgt

SEQ ID NO: 77 - RNA, 20 nt, synthetic construct
  source 1..20: mol_type = other RNA
gtagttggagctggtagcgt

SEQ ID NO: 78 - RNA, 20 nt, synthetic construct
  source 1..20: mol_type = other RNA
gtagttggagctggttgcgt

SEQ ID NO: 79 - RNA, 20 nt, synthetic construct
  source 1..20: mol_type = other RNA
gtagttggagctgggggcgt

SEQ ID NO: 80 - RNA, 20 nt, synthetic construct
  source 1..20: mol_type = other RNA
gtagttggagctggcggcgt

SEQ ID NO: 81 - RNA, 20 nt, synthetic construct
  source 1..20: mol_type = other RNA
gtagttggagctgatggcgt

SEQ ID NO: 82 - RNA, 20 nt, synthetic construct
  source 1..20: mol_type = other RNA
gtagttggagctgttggcgt

SEQ ID NO: 83 - RNA, 20 nt, synthetic construct
  source 1..20: mol_type = other RNA
gtagttggagctagtggcgt

SEQ ID NO: 84 - RNA, 20 nt, synthetic construct
  source 1..20: mol_type = other RNA
gtagttggagcttgtggcgt

SEQ ID NO: 85 - RNA, 20 nt, synthetic construct
  source 1..20: mol_type = other RNA
gtagttggagcgggtggcgt

SEQ ID NO: 86 - RNA, 20 nt, synthetic construct
  source 1..20: mol_type = other RNA
gtagttggagccggtggcgt

SEQ ID NO: 87 - RNA, 20 nt, synthetic construct
  source 1..20: mol_type = other RNA
gtagttggagatggtggcgt

SEQ ID NO: 88 - RNA, 20 nt, synthetic construct
  source 1..20: mol_type = other RNA
gtagttggagttggtggcgt

SEQ ID NO: 89 - RNA, 20 nt, synthetic construct
  source 1..20: mol_type = other RNA
gtagttggaactggtggcgt

SEQ ID NO: 90 - RNA, 20 nt, synthetic construct
  source 1..20: mol_type = other RNA
gtagttggatctggtggcgt

SEQ ID NO: 91 - RNA, 20 nt, synthetic construct
  source 1..20: mol_type = other RNA
gtagttggggctggtggcgt

SEQ ID NO: 92 - RNA, 20 nt, synthetic construct
  source 1..20: mol_type = other RNA
gtagttggcgctggtggcgt

SEQ ID NO: 93 - RNA, 20 nt, synthetic construct
  source 1..20: mol_type = other RNA
gtagttgaagctggtggcgt

SEQ ID NO: 94 - RNA, 20 nt, synthetic construct
  source 1..20: mol_type = other RNA
gtagttgtagctggtggcgt

SEQ ID NO: 95 - RNA, 20 nt, synthetic construct
  source 1..20: mol_type = other RNA
gtagttagagctggtggcgt

SEQ ID NO: 96 - RNA, 20 nt, synthetic construct
  source 1..20: mol_type = other RNA
gtagtttgagctggtggcgt

SEQ ID NO: 97 - RNA, 20 nt, synthetic construct
  source 1..20: mol_type = other RNA
gtagtgggagctggtggcgt

SEQ ID NO: 98 - RNA, 20 nt, synthetic construct
  source 1..20: mol_type = other RNA
gtagtcggagctggtggcgt

SEQ ID NO: 99 - RNA, 20 nt, synthetic construct
  source 1..20: mol_type = other RNA
gtaggtggagctggtggcgt

SEQ ID NO: 100 - RNA, 20 nt, synthetic construct
  source 1..20: mol_type = other RNA
gtagctggagctggtggcgt

SEQ ID NO: 101 - RNA, 20 nt, synthetic construct
  source 1..20: mol_type = other RNA
gtaattggagctggtggcgt

SEQ ID NO: 102 - RNA, 20 nt, synthetic construct
  source 1..20: mol_type = other RNA
gtatttggagctggtggcgt

SEQ ID NO: 103 - RNA, 20 nt, synthetic construct
  source 1..20: mol_type = other RNA
gtggttggagctggtggcgt

SEQ ID NO: 104 - RNA, 20 nt, synthetic construct
  source 1..20: mol_type = other RNA
gtcgttggagctggtggcgt

SEQ ID NO: 105 - RNA, 20 nt, synthetic construct
  source 1..20: mol_type = other RNA
ttggacatactggatacagc

SEQ ID NO: 106 - RNA, 20 nt, synthetic construct
  source 1..20: mol_type = other RNA
ttggacatactggatacagg

SEQ ID NO: 107 - RNA, 20 nt, synthetic construct
  source 1..20: mol_type = other RNA
ttggacatactggatacaga

SEQ ID NO: 108 - RNA, 20 nt, synthetic construct
  source 1..20: mol_type = other RNA
ttggacatactggatacagt

SEQ ID NO: 109 - RNA, 20 nt, synthetic construct
  source 1..20: mol_type = other RNA
ttggacatactggatacacc

SEQ ID NO: 110 - RNA, 20 nt, synthetic construct
  source 1..20: mol_type = other RNA
ttggacatactggatacaac

SEQ ID NO: 111 - RNA, 20 nt, synthetic construct
  source 1..20: mol_type = other RNA
ttggacatactggatacatc

SEQ ID NO: 112 - RNA, 20 nt, synthetic construct
  source 1..20: mol_type = other RNA
ttggacatactggatactgc

SEQ ID NO: 113 - RNA, 20 nt, synthetic construct
  source 1..20: mol_type = other RNA
ttggacatactggatacggc

SEQ ID NO: 114 - RNA, 20 nt, synthetic construct
  source 1..20: mol_type = other RNA
ttggacatactggataccgc

SEQ ID NO: 115 - RNA, 20 nt, synthetic construct
  source 1..20: mol_type = other RNA
ttggacatactggatagagc

SEQ ID NO: 116 - RNA, 20 nt, synthetic construct
  source 1..20: mol_type = other RNA
ttggacatactggataaagc

SEQ ID NO: 117 - RNA, 20 nt, synthetic construct
  source 1..20: mol_type = other RNA
ttggacatactggatatagc

SEQ ID NO: 118 - RNA, 20 nt, synthetic construct
  source 1..20: mol_type = other RNA
ttggacatactggattcagc

SEQ ID NO: 119 - RNA, 20 nt, synthetic construct
  source 1..20: mol_type = other RNA
ttggacatactggatgcagc

SEQ ID NO: 120 - RNA, 20 nt, synthetic construct
  source 1..20: mol_type = other RNA
ttggacatactggatccagc

SEQ ID NO: 121 - RNA, 20 nt, synthetic construct
  source 1..20: mol_type = other RNA
ttggacatactggaaacagc

SEQ ID NO: 122 - RNA, 20 nt, synthetic construct
  source 1..20: mol_type = other RNA
ttggacatactggagacagc

SEQ ID NO: 123 - RNA, 20 nt, synthetic construct
  source 1..20: mol_type = other RNA
ttggacatactggacacagc

SEQ ID NO: 124 - RNA, 20 nt, synthetic construct
  source 1..20: mol_type = other RNA
ttggacatactggttacagc

SEQ ID NO: 125 - RNA, 20 nt, synthetic construct
  source 1..20: mol_type = other RNA
ttggacatactgggtacagc

SEQ ID NO: 126 - RNA, 20 nt, synthetic construct
  source 1..20: mol_type = other RNA
ttggacatactggctacagc

SEQ ID NO: 127 - RNA, 20 nt, synthetic construct
  source 1..20: mol_type = other RNA
ttggacatactgcatacagc

SEQ ID NO: 128 - RNA, 20 nt, synthetic construct
  source 1..20: mol_type = other RNA
ttggacatactgaatacagc

SEQ ID NO: 129 - RNA, 20 nt, synthetic construct
  source 1..20: mol_type = other RNA
ttggacatactgtatacagc

SEQ ID NO: 130 - RNA, 20 nt, synthetic construct
  source 1..20: mol_type = other RNA
ttggacatactcgatacagc

SEQ ID NO: 131 - RNA, 20 nt, synthetic construct
  source 1..20: mol_type = other RNA
ttggacatactagatacagc

SEQ ID NO: 132 - RNA, 20 nt, synthetic construct
  source 1..20: mol_type = other RNA
ttggacatacttgatacagc

SEQ ID NO: 133 - RNA, 20 nt, synthetic construct
  source 1..20: mol_type = other RNA
ttggacatacaggatacagc

SEQ ID NO: 134 - RNA, 20 nt, synthetic construct
  source 1..20: mol_type = other RNA
ttggacatacgggatacagc

SEQ ID NO: 135 - RNA, 20 nt, synthetic construct
  source 1..20: mol_type = other RNA
ttggacataccggatacagc

SEQ ID NO: 136 - RNA, 20 nt, synthetic construct
  source 1..20: mol_type = other RNA
ttggacatagtggatacagc

SEQ ID NO: 137 - RNA, 20 nt, synthetic construct
  source 1..20: mol_type = other RNA
ttggacataatggatacagc

SEQ ID NO: 138 - RNA, 20 nt, synthetic construct
  source 1..20: mol_type = other RNA
ttggacatattggatacagc

SEQ ID NO: 139 - RNA, 20 nt, synthetic construct
  source 1..20: mol_type = other RNA
ttggacattctggatacagc

SEQ ID NO: 140 - RNA, 20 nt, synthetic construct
  source 1..20: mol_type = other RNA
ttggacatgctggatacagc

SEQ ID NO: 141 - RNA, 20 nt, synthetic construct
  source 1..20: mol_type = other RNA
ttggacatcctggatacagc

SEQ ID NO: 142 - RNA, 20 nt, synthetic construct
  source 1..20: mol_type = other RNA
ttggacaaactggatacagc

SEQ ID NO: 143 - RNA, 20 nt, synthetic construct
  source 1..20: mol_type = other RNA
ttggacagactggatacagc

SEQ ID NO: 144 - RNA, 20 nt, Streptococcus pyogenes
  source 1..20: mol_type = other RNA
ttggacacactggatacagc

SEQ ID NO: 145 - RNA, 20 nt, synthetic construct
  source 1..20: mol_type = other RNA
ttggacttactggatacagc

SEQ ID NO: 146 - RNA, 20 nt, synthetic construct
  source 1..20: mol_type = other RNA
ttggacgtactggatacagc

SEQ ID NO: 147 - RNA, 20 nt, synthetic construct
  source 1..20: mol_type = other RNA
ttggacctactggatacagc

SEQ ID NO: 148 - RNA, 20 nt, synthetic construct
  source 1..20: mol_type = other RNA
ttggagatactggatacagc

SEQ ID NO: 149 - RNA, 20 nt, synthetic construct
  source 1..20: mol_type = other RNA
ttggaaatactggatacagc

SEQ ID NO: 150 - RNA, 20 nt, synthetic construct
  source 1..20: mol_type = other RNA
ttggatatactggatacagc

SEQ ID NO: 151 - RNA, 20 nt, synthetic construct
  source 1..20: mol_type = other RNA
ttggtcatactggatacagc

SEQ ID NO: 152 - RNA, 20 nt, synthetic construct
  source 1..20: mol_type = other RNA
ttgggcatactggatacagc

SEQ ID NO: 153 - RNA, 20 nt, synthetic construct
  source 1..20: mol_type = other RNA
ttggccatactggatacagc

SEQ ID NO: 154 - RNA, 20 nt, synthetic construct
  source 1..20: mol_type = other RNA
ttgcacatactggatacagc

SEQ ID NO: 155 - RNA, 20 nt, synthetic construct
  source 1..20: mol_type = other RNA
ttgaacatactggatacagc

SEQ ID NO: 156 - RNA, 20 nt, synthetic construct
  source 1..20: mol_type = other RNA
ttgtacatactggatacagc

SEQ ID NO: 157 - RNA, 20 nt, synthetic construct
  source 1..20: mol_type = other RNA
ttcgacatactggatacagc

SEQ ID NO: 158 - RNA, 20 nt, synthetic construct
  source 1..20: mol_type = other RNA
ttagacatactggatacagc

SEQ ID NO: 159 - RNA, 20 nt, synthetic construct
  source 1..20: mol_type = other RNA
tttgacatactggatacagc

SEQ ID NO: 160 - RNA, 20 nt, synthetic construct
  source 1..20: mol_type = other RNA
taggacatactggatacagc

SEQ ID NO: 161 - RNA, 20 nt, synthetic construct
  source 1..20: mol_type = other RNA
tgggacatactggatacagc

SEQ ID NO: 162 - RNA, 20 nt, synthetic construct
  source 1..20: mol_type = other RNA
tcggacatactggatacagc

SEQ ID NO: 163 - RNA, 20 nt, synthetic construct
  source 1..20: mol_type = other RNA
atggacatactggatacagc

SEQ ID NO: 164 - RNA, 20 nt, synthetic construct
  source 1..20: mol_type = other RNA
gtggacatactggatacagc

SEQ ID NO: 165 - RNA, 20 nt, synthetic construct
  source 1..20: mol_type = other RNA
ctggacatactggatacagc

SEQ ID NO: 166 - RNA, 20 nt, synthetic construct
  source 1..20: mol_type = other RNA
cagattttgggctggccaaa

SEQ ID NO: 167 - RNA, 20 nt, synthetic construct
  source 1..20: mol_type = other RNA
cagattttgggctggccaat

SEQ ID NO: 168 - RNA, 20 nt, synthetic construct
  source 1..20: mol_type = other RNA
cagattttgggctggccaag

SEQ ID NO: 169 - RNA, 20 nt, synthetic construct
  source 1..20: mol_type = other RNA
cagattttgggctggccaac

SEQ ID NO: 170 - RNA, 20 nt, synthetic construct
  source 1..20: mol_type = other RNA
cagattttgggctggccata

SEQ ID NO: 171 - RNA, 20 nt, synthetic construct
  source 1..20: mol_type = other RNA
cagattttgggctggccaga

SEQ ID NO: 172 - RNA, 20 nt, synthetic construct
  source 1..20: mol_type = other RNA
cagattttgggctggccaca

SEQ ID NO: 173 - RNA, 20 nt, synthetic construct
  source 1..20: mol_type = other RNA
cagattttgggctggcctaa

SEQ ID NO: 174 - RNA, 20 nt, synthetic construct
  source 1..20: mol_type = other RNA
cagattttgggctggccgaa

SEQ ID NO: 175 - RNA, 20 nt, synthetic construct
  source 1..20: mol_type = other RNA
cagattttgggctggcccaa

SEQ ID NO: 176 - RNA, 20 nt, synthetic construct
  source 1..20: mol_type = other RNA
cagattttgggctggcgaaa

SEQ ID NO: 177 - RNA, 20 nt, synthetic construct
  source 1..20: mol_type = other RNA
cagattttgggctggcaaaa

SEQ ID NO: 178 - RNA, 20 nt, synthetic construct
  source 1..20: mol_type = other RNA
cagattttgggctggctaaa

SEQ ID NO: 179 - RNA, 20 nt, synthetic construct
  source 1..20: mol_type = other RNA
cagattttgggctgggcaaa

SEQ ID NO: 180 - RNA, 20 nt, synthetic construct
  source 1..20: mol_type = other RNA
cagattttgggctggacaaa

SEQ ID NO: 181 - RNA, 20 nt, synthetic construct
  source 1..20: mol_type = other RNA
cagattttgggctggtcaaa

SEQ ID NO: 182 - RNA, 20 nt, synthetic construct
  source 1..20: mol_type = other RNA
cagattttgggctgcccaaa

SEQ ID NO: 183 - RNA, 20 nt, synthetic construct
  source 1..20: mol_type = other RNA
cagattttgggctgaccaaa

SEQ ID NO: 184 - RNA, 20 nt, synthetic construct
  source 1..20: mol_type = other RNA
cagattttgggctgtccaaa

SEQ ID NO: 185 - RNA, 20 nt, synthetic construct
  source 1..20: mol_type = other RNA
cagattttgggctcgccaaa

SEQ ID NO: 186 - RNA, 20 nt, synthetic construct
  source 1..20: mol_type = other RNA
cagattttgggctagccaaa

SEQ ID NO: 187 - RNA, 20 nt, synthetic construct
  source 1..20: mol_type = other RNA
cagattttgggcttgccaaa

SEQ ID NO: 188 - RNA, 20 nt, synthetic construct
  source 1..20: mol_type = other RNA
cagattttgggcaggccaaa

SEQ ID NO: 189 - RNA, 20 nt, synthetic construct
  source 1..20: mol_type = other RNA
cagattttgggcgggccaaa

SEQ ID NO: 190 - RNA, 20 nt, synthetic construct
  source 1..20: mol_type = other RNA
cagattttgggccggccaaa

SEQ ID NO: 191 - RNA, 20 nt, synthetic construct
  source 1..20: mol_type = other RNA
cagattttggggtggccaaa

SEQ ID NO: 192 - RNA, 20 nt, synthetic construct
  source 1..20: mol_type = other RNA
cagattttgggatggccaaa

SEQ ID NO: 193 - RNA, 20 nt, synthetic construct
  source 1..20: mol_type = other RNA
cagattttgggttggccaaa

SEQ ID NO: 194 - RNA, 20 nt, synthetic construct
  source 1..20: mol_type = other RNA
cagattttggcctggccaaa

SEQ ID NO: 195 - RNA, 20 nt, synthetic construct
  source 1..20: mol_type = other RNA
cagattttggactggccaaa

SEQ ID NO: 196 - RNA, 20 nt, synthetic construct
  source 1..20: mol_type = other RNA
cagattttggtctggccaaa

SEQ ID NO: 197 - RNA, 20 nt, synthetic construct
  source 1..20: mol_type = other RNA
cagattttgcgctggccaaa

SEQ ID NO: 198 - RNA, 20 nt, synthetic construct
  source 1..20: mol_type = other RNA
cagattttgagctggccaaa

SEQ ID NO: 199 - RNA, 20 nt, synthetic construct
  source 1..20: mol_type = other RNA
cagattttgtgctggccaaa

SEQ ID NO: 200 - RNA, 20 nt, synthetic construct
  source 1..20: mol_type = other RNA
cagattttcggctggccaaa

SEQ ID NO: 201 - RNA, 20 nt, synthetic construct
  source 1..20: mol_type = other RNA
cagattttaggctggccaaa

SEQ ID NO: 202 - RNA, 20 nt, synthetic construct
  source 1..20: mol_type = other RNA
cagatttttggctggccaaa

SEQ ID NO: 203 - RNA, 20 nt, synthetic construct
  source 1..20: mol_type = other RNA
cagatttagggctggccaaa

SEQ ID NO: 204 - RNA, 20 nt, synthetic construct
  source 1..20: mol_type = other RNA
cagatttggggctggccaaa

SEQ ID NO: 205 - RNA, 20 nt, synthetic construct
  source 1..20: mol_type = other RNA
cagatttcgggctggccaaa

SEQ ID NO: 206 - RNA, 20 nt, synthetic construct
  source 1..20: mol_type = other RNA
cagattatgggctggccaaa

SEQ ID NO: 207 - RNA, 20 nt, synthetic construct
  source 1..20: mol_type = other RNA
cagattgtgggctggccaaa

SEQ ID NO: 208 - RNA, 20 nt, synthetic construct
  source 1..20: mol_type = other RNA
cagattctgggctggccaaa

SEQ ID NO: 209 - RNA, 20 nt, synthetic construct
  source 1..20: mol_type = other RNA
cagatattgggctggccaaa

SEQ ID NO: 210 - RNA, 20 nt, synthetic construct
  source 1..20: mol_type = other RNA
cagatgttgggctggccaaa

SEQ ID NO: 211 - RNA, 20 nt, synthetic construct
  source 1..20: mol_type = other RNA
cagatcttgggctggccaaa

SEQ ID NO: 212 - RNA, 20 nt, synthetic construct
  source 1..20: mol_type = other RNA
cagaatttgggctggccaaa

SEQ ID NO: 213 - RNA, 20 nt, synthetic construct
  source 1..20: mol_type = other RNA
cagagtttgggctggccaaa

SEQ ID NO: 214 - RNA, 20 nt, synthetic construct
  source 1..20: mol_type = other RNA
cagactttgggctggccaaa

SEQ ID NO: 215 - RNA, 20 nt, synthetic construct
  source 1..20: mol_type = other RNA
cagtttttgggctggccaaa

SEQ ID NO: 216 - RNA, 20 nt, synthetic construct
  source 1..20: mol_type = other RNA
caggttttgggctggccaaa

SEQ ID NO: 217 - RNA, 20 nt, synthetic construct
  source 1..20: mol_type = other RNA
cagcttttgggctggccaaa

SEQ ID NO: 218 - RNA, 20 nt, synthetic construct
  source 1..20: mol_type = other RNA
cacattttgggctggccaaa

SEQ ID NO: 219 - RNA, 20 nt, synthetic construct
  source 1..20: mol_type = other RNA
caaattttgggctggccaaa

SEQ ID NO: 220 - RNA, 20 nt, synthetic construct
  source 1..20: mol_type = other RNA
catattttgggctggccaaa

SEQ ID NO: 221 - RNA, 20 nt, synthetic construct
  source 1..20: mol_type = other RNA
ctgattttgggctggccaaa

SEQ ID NO: 222 - RNA, 20 nt, synthetic construct
  source 1..20: mol_type = other RNA
cggattttgggctggccaaa

SEQ ID NO: 223 - RNA, 20 nt, synthetic construct
  source 1..20: mol_type = other RNA
ccgattttgggctggccaaa

SEQ ID NO: 224 - RNA, 20 nt, synthetic construct
  source 1..20: mol_type = other RNA
gagattttgggctggccaaa

SEQ ID NO: 225 - RNA, 20 nt, synthetic construct
  source 1..20: mol_type = other RNA
aagattttgggctggccaaa

SEQ ID NO: 226 - RNA, 20 nt, synthetic construct
  source 1..20: mol_type = other RNA
tagattttgggctggccaaa

SEQ ID NO: 227 - RNA, 20 nt, synthetic construct
  source 1..20: mol_type = other RNA
agagaagcaacatctccgaa

SEQ ID NO: 228 - RNA, 20 nt, synthetic construct
  source 1..20: mol_type = other RNA
ggttgtccacgctggccatc

SEQ ID NO: 229 - RNA, 20 nt, synthetic construct
  source 1..20: mol_type = other RNA
ggcatgagctgcgtgatgag

SEQ ID NO: 230 - RNA, 20 nt, synthetic construct
  source 1..20: mol_type = other RNA
cagattttgggctggccaaa

SEQ ID NO: 231 - RNA, 20 nt, synthetic construct
  source 1..20: mol_type = other RNA
attgaaacatcagcaaagac

SEQ ID NO: 232 - RNA, 20 nt, synthetic construct
  source 1..20: mol_type = other RNA
tggttggagcaggtggtgtt

SEQ ID NO: 233 - RNA, 20 nt, synthetic construct
  source 1..20: mol_type = other RNA
ggatacagctggacaagaag

SEQ ID NO: 234 - RNA, 20 nt, synthetic construct
  source 1..20: mol_type = other RNA
gccaggagggggctggtgca

SEQ ID NO: 235 - RNA, 20 nt, synthetic construct
  source 1..20: mol_type = other RNA
ggcgctgcccccaccatgag

SEQ ID NO: 236 - RNA, 20 nt, synthetic construct
  source 1..20: mol_type = other RNA
ggatgggcctccggttcatg

SEQ ID NO: 237 - RNA, 20 nt, synthetic construct
  source 1..20: mol_type = other RNA
gtaatctactgggacggaac

SEQ ID NO: 238 - RNA, 20 nt, synthetic construct
  source 1..20: mol_type = other RNA
tctgtgcgccggtctctccc

SEQ ID NO: 239 - RNA, 20 nt, synthetic construct
  source 1..20: mol_type = other RNA
agggagcactaagcgaggta

SEQ ID NO: 240 - RNA, 20 nt, synthetic construct
  source 1..20: mol_type = other RNA
atgatgcacatcatggtggc

SEQ ID NO: 241 - RNA, 20 nt, synthetic construct
  source 1..20: mol_type = other RNA
ttatcttttcagttcaatgc

SEQ ID NO: 242 - RNA, 20 nt, synthetic construct
  source 1..20: mol_type = other RNA
gtctagctacagtgaaatct

SEQ ID NO: 243 - RNA, 20 nt, synthetic construct
  source 1..20: mol_type = other RNA
gtctggatccattttgtgga

SEQ ID NO: 244 - protein, 1368 aa, synthetic construct [sequence omitted]

SEQ ID NO: 245 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 246 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEALYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 247 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 248 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPAYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 249 - DNA, 20 bp, synthetic construct
  source 1..20: mol_type = other DNA
gtagttggagctggtggcgt

SEQ ID NO: 250 - DNA, 20 bp, synthetic construct
  source 1..20: mol_type = other DNA
cagattttgggctggccaaa

SEQ ID NO: 251 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVADQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 252 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNAKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 253 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQAASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 254 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSAKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 255 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQARYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 256 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNAGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 257 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDAKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 258 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRAIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 259 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKAFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 260 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGASLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 261 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLADRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 262 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDARKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 263 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRAKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 264 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKAALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 265 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALEAISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 266 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFADKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 267 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDANNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 268 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNAIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 269 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIAEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 270 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAAGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 271 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIAQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 272 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEAVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 273 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAAGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 274 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDANTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 275 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTADIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 276 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKALVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 277 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVAKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 278 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRAKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 279 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKAAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 280 - protein, 20 aa, synthetic construct
TTGGACATACTGGATACAGC

SEQ ID NO: 281 - DNA, 2928 bp, synthetic construct
  source 1..2928: mol_type = other DNA
gggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgattctgaattagctgtatcgtcaaggcactcttgcctacgccaccagctccaactaccacaagtttatattcagtcattttcagcaggccttataattctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagt

SEQ ID NO: 282 - DNA, 2928 bp, synthetic construct
  source 1..2928: mol_type = other DNA
gggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctactagagaacccactgcttactggcttatcgaaattaatacgactcactatagggagacccaagctttcgcctagtcctcatgtattggtctctcatggcactgtcctcttcttgtccagctgtatccagtatgtccaacaaacaggtttcaccatctataaccactttctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagt

SEQ ID NO: 283 - DNA, 2936 bp, synthetic construct
  source 1..2936: mol_type = other DNA
gggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcttggtgcaccgcgacctggcagccaggaacgtactggtgaaaacaccgcagcatgtcaagatcacagattttgggctggccaaacggctgggtgcggaagagaaagatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagt

SEQ ID NO: 284 - DNA, 115 bp, synthetic construct
  source 1..115: mol_type = other DNA
actctggatcccagaaggtgagaaagttaaaattcccgtcgctatcaaggaattaagagaagcaacatctccgaaaggcaacaaggaaatcctcgatgtgagtttctgctttgct

SEQ ID NO: 285 - DNA, 100 bp, synthetic construct
  source 1..100: mol_type = other DNA
actctggatcccagaaggtgagaaagttaaaattcccgtcgctatcaagacatctccgaaaggcaacaaggaaatcctcgatgtgagtttctgctttgct

SEQ ID NO: 286 - DNA, 100 bp, synthetic construct
  source 1..100: mol_type = other DNA
gtgccgcctgctgggcatctgcctcacctccaccgtgcagctcatcacgcagctcatgcccttcggctgcctcctggactatgtccgggaacacaaagac

SEQ ID NO: 287 - DNA, 100 bp, synthetic construct
  source 1..100: mol_type = other DNA
gtgccgcctgctgggcatctgcctcacctccaccgtgcagctcatcatgcagctcatgcccttcggctgcctcctggactatgtccgggaacacaaagac

SEQ ID NO: 288 - DNA, 108 bp, synthetic construct
  source 1..108: mol_type = other DNA
cttggtgcaccgcgacctggcagccaggaacgtactggtgaaaacaccgcagcatgtcaagatcacagattttgggctggccaaacggctgggtgcggaagagaaaga

SEQ ID NO: 289 - DNA, 108 bp, synthetic construct
  source 1..108: mol_type = other DNA
cttggtgcaccgcgacctggcagccaggaacgtactggtgaaaacaccgcagcatgtcaagatcacagattttgggcgggccaaacggctgggtgcggaagagaaaga

SEQ ID NO: 290 - DNA, 100 bp, synthetic construct
  source 1..100: mol_type = other DNA
atgattctgaattagctgtatcgtcaaggcactcttgcctacgccaccagctccaactaccacaagtttatattcagtcattttcagcaggccttataat

SEQ ID NO: 291 - DNA, 100 bp, synthetic construct
  source 1..100: mol_type = other DNA
atgattctgaattagctgtatcgtcaaggcactcttgcctacgccatcagctccaactaccacaagtttatattcagtcattttcagcaggccttataat

SEQ ID NO: 292 - skipped

SEQ ID NO: 293 - RNA, 20 nt, synthetic construct
  source 1..20: mol_type = other RNA
ggagttggagctggtggcgt

SEQ ID NO: 294 - RNA, 20 nt, synthetic construct
  source 1..20: mol_type = other RNA
gcagttggagctggtggcgt

SEQ ID NO: 295 - RNA, 20 nt, synthetic construct
  source 1..20: mol_type = other RNA
atagttggagctggtggcgt

SEQ ID NO: 296 - RNA, 20 nt, synthetic construct
  source 1..20: mol_type = other RNA
ttagttggagctggtggcgt